(19) United States
(12) Patent Application Publication
(10) Pub. No.: US 2026/0335433 A1
(43) Pub. Date: Oct. 29, 2026

(54) ENRICHMENT OF ENGINEERED IMMUNE CELLS

(71) Applicant: Biosceptre (Aust) Pty Ltd, North Ryde, New South Wales (AU)

(72) Inventors: Patrick Schlegel, North Ryde, New South Wales (AU); Coralie Werbrouck, North Ryde, New South Wales (AU)

(21) Appl. No.: 19/110,350

(22) PCT Filed: Sep. 14, 2023
(86) PCT No.: PCT/AU2023/050890

§ 371 Date: Mar. 10, 2025

(30) Foreign Application Priority Data
Jun. 20, 2022 (AU) 2023901949
Sep. 14, 2022 (AU) 2022902655

(51) Int. Cl. A61K 40/11; A61K 40/42; C07K 16/28

(52) U.S. Cl. CPC A61K 40/11; A61K 40/4202; C07K 16/28

(57) ABSTRACT

The invention relates to fusion proteins and uses thereof for obtaining and isolating enriched populations of immune cells. Exemplified fusion proteins include those comprising a dysfunctional purinergic P2X7 receptor (P2X7R) epitope moiety and a CN Fc region of an antibody.

### FIELD OF THE INVENTION

The present invention relates to methods for enriching a population of immune cells, and compositions and molecules for performing same.

### RELATED APPLICATIONS

This application claims priority from Australian provisional applications AU 2022902655 and AU 2023901949, the entire contents of which are hereby incorporated by reference.

### BACKGROUND OF THE INVENTION

Cell therapy to treat cancer and other disease states is a rapidly growing field. The development of immune effector cells such as T cells expressing chimeric antigen receptors (CARs) has revolutionised adoptive cell therapies.

The potential of this approach has been demonstrated in clinical trials, wherein CAR T cells were infused into adult and paediatric patients with B-cell malignancies, neuroblastoma, and sarcoma. To date, over 500 clinical trials have emerged worldwide, designed at testing the efficacy of CAR T cells targeted to bind 64 different tumour associated antigens. Among these, three CD19-specific CAR T cell products have been approved for the treatment of acute lymphoblastic leukaemia (ALL), large B cell lymphoma and mantle cell lymphoma. To date, most of the success with CAR T therapies has been observed in the context of so-called “liquid” tumours, or where the CARs are directed to CD19, CD22 or the B cell maturation antigen (BCMA).

Human derived T lymphocytes engineered to express CARs, which are expanded in in vitro culture and then infused into patients, exert robust cytotoxicity after tumour antigen recognition and subsequent activation. Various factors in the manufacture and administration of these cells contribute the in vivo persistence and durable antitumour effects of the CAR T cells.

In order to prepare CAR T cells for infusion into a patient, it is first necessary to genetically modify a population of T cells to express the relevant CAR. This results in a mixed population of T cells, some of which express the CAR, and some of which do not. In order to maximise efficacy of the CAR T treatment, it is desirable to obtain a population of T cells which is enriched for cells which express the CAR.

A key issue with CAR-engineered T cell therapies (and cell therapies, in general) is the low transduction efficiency of T cells with CAR-expressing constructs. For instance, peripheral blood T cells that are often the target of CAR gene therapy typically have transduction efficiencies less than 50%, often 10-20%. As a result, production of sufficient cell numbers for therapy requires an increase in the scale of patient cell collection as well as more extensive ex vivo T-cell selection and expansion. This contributes to CAR T cell therapies high manufacturing costs.

Therefore, there is a need for improved and alternative approaches for preparing a population of immune cells prior to cell therapy.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention finds particular application in the preparation of a population of genetically modified immune cells that are engineered to bind to dysfunctional P2X₇ receptor on a cancer cell. Accordingly, in a first aspect, there is provided a fusion protein comprising:
(i) a dysfunctional P2X₇ receptor epitope moiety; and
(ii) an Fc region of an antibody.

The dysfunctional P2X₇ receptor epitope moiety may comprise a peptide that comprises any amino acid sequence which is derived from the dysfunctional P2X₇ receptor although preferably, comprises the sequence of an epitope which is found on dysfunctional P2X₇ receptor but not on functional P2X₇ receptor.

In a preferred embodiment, the amino acid sequence of the dysfunctional P2X₇ receptor epitope moiety comprises or consists at least of the amino acid sequence as set forth in SEQ ID NO: 14. In an especially preferred embodiment, the moiety comprises at least the sequence as set forth in SEQ ID NO: 7 or 9.

In any embodiment, the dysfunctional P2X₇ receptor epitope moiety comprises an amino acid sequence as set forth in any of SEQ ID NOs: 7 to 69 or 122 or sequences at least 80%, at least 81%, at least 82%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical thereto, provided that the sequences comprise at least the sequence set forth in SEQ ID NO: 14 or 7 or 9.

The present invention provides a fusion protein comprising:
(i) a peptide; and
(ii) an Fc region of an antibody,
   wherein the peptide comprises or consists of the amino acid sequence of SEQ ID NO: 7 (preferably the amino acid sequence of SEQ ID NO: 14). Optionally, the peptide comprises or consists of the amino acid sequence of any of SEQ ID NOs: 7 to 69 or 122 or sequences at least 80%, at least 81%, at least 82%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical thereto, provided that the sequences comprise at least the sequence set forth in SEQ ID NO: 14 or 7 or 9.

In any embodiment, the Fc region of an antibody is an Fc region of an IgG, IgA, IgD, IgE, or IgM. Preferably the Fc region is from an IgG antibody, such as an IgG1, an IgG2, an IgG2b, an IgG3 or an IgG4 antibody.

Preferably, the Fc region of the fusion protein comprises two heavy chain fragments, more preferably the CH2 and CH3 domains of said heavy chain.

In a preferred embodiment, the Fc region of the fusion protein comprises one or more amino acid substitutions compared to naturally occurring Fc sequences, which prevent or reduce the ability of the Fc region to homodimerise. Preferably, the amino acid substitutions comprise one or more substitutions of the cysteine residues so as to prevent the formation of disulphide bonds between Fc molecules. The cysteine residues of the Fc region may be substituted to any other amino acid residue, optionally to glycine, serine, alanine, lysine and glutamic acid, preferably glycine or serine.

The cysteine residues for substitution are preferably one or more of the cysteine residues located in the region of the Fc region which corresponds to the hinge region of an immunoglobulin. Examples of the IgG1 hinge regions, and variations thereof including cysteine to serine substitutions are provided herein in Table 3. The hinge region of an immunoglobulin (eg of IgG1) comprises three cysteine residues (which are number C220, C226 and C229 according to EU numbering. Accordingly, in any embodiment, at least one, at least two, or all three of the cysteine residues in the immunoglobulin hinge region are substituted. Preferably, at least two or all three of the cysteine residues are substituted. More preferably, all cysteine residues in the Fc region, such as the hinge region, are substituted. In particularly preferred embodiments, at least one of C226 and C229 are substituted, preferably wherein both C226 and C229 are substituted.

Accordingly, in preferred embodiments, the fusion protein comprises a hinge region for linking the a peptide as described herein (eg a dysfunctional P2X₇ receptor epitope moiety) and Fc region of an antibody, wherein the hinge region comprises an amino acid sequence that corresponds to any of the sequences set forth in SEQ ID NOs: 76 to 113, or 136 to 137 or 141 or 142.

Further still, the Fc region preferably comprises one or more amino acid substitutions for reducing affinity for the Fc receptor (FcR, including any of FcγRI, FcγRII and FcγRIII) and thereby reducing the ability of the fusion protein to elicit antibody-dependent cell-mediated toxicity (ADCC). Such substitutions are well known in the art and include, but are not limited to the “DANA” and “LALA” amino acid substitutions and variations thereof, as further defined herein. In further embodiments, the Fc region may also comprise substitutions which abrogate recruitment of complement C1q. Such mutations are also well known in the art and are further described herein. Further still, the Fc region may comprise substitutions to reduce serum half-life (through attenuating or reducing capacity to bind to the FcRN receptor). Relevant amino acid substitutions for altering effector function, serum half-life and aggregation are well known to the skilled person and further described herein, including as exemplified in Table 1.

The present invention also provides a heterodimeric asymmetric molecule comprising a fusion protein as described herein (preferably comprising a peptide of SEQ ID NO: 7 or 14, or variations thereof as exemplified in any of SEQ ID NOs: 2 to 69) and an Fc region of an antibody, and further comprising an Fc region of an antibody that does not comprise the peptide. Such asymmetric heterodimeric molecules may be obtained using the knob-in-hole technology, as further described herein, for facilitating dimerisation of non-identical Fc regions.

The present invention provides a heterodimeric asymmetric molecule or monomeric fusion protein for use in accordance with any of the methods further described herein, and which have application in methods for enriching immune cells which comprise exogenous cell surface receptors for binding to tumour specific or tumour associated antigens, and comprising an intracellular signalling domain (eg chimeric antigen receptors, including expressed on T cells). It will be appreciated that such molecules or fusion proteins comprise a single amino acid sequence capable of being bound by the exogenous immune cell surface receptor so as to minimise the likelihood that the molecules/fusion proteins cross-link multiple immune cells and thereby cause unwanted activation thereof.

More specifically, there is provided a monomeric fusion protein comprising i) an amino acid sequence that is recognised or capable of being bound by an antigen recognition domain of an exogenous cell surface receptor comprising an intracellular signalling domain (such as a chimeric antigen receptor), and ii) an Fc region of an antibody. Preferably the amino acid sequence of the Fc region of the antibody is unable to form a homodimer with another Fc region of an antibody.

Further, there is provided a heterodimeric asymmetric molecule comprising i) an amino acid sequence that is recognised or capable of being bound by an antigen recognition domain of an exogenous cell surface receptor comprising an intracellular signalling domain (eg chimeric antigen receptors, including expressed on T cells), wherein the amino acid sequence is joined to a first Fc region of an antibody; ii) a second Fc region of an antibody capable of forming a heterodimer with the first Fc region.

In preferred embodiments, the fusion protein has an affinity for FcR that is less than about 250 nM, preferably less than 500 nM, less than 1000 nM, most preferably less than 2000 nM.

In any embodiment, the fusion protein may comprise the amino acid sequence of a fusion protein as set forth in any of SEQ ID NOs: 145 to 158, 160 and 161.

Preferably, the fusion protein or the heterodimeric asymmetric molecule, consists or consists essentially of the peptide and an Fc region of an antibody, such that the fusion protein or heterodimeric asymmetric molecule does not comprise an antigen binding domain of an antibody (ie such that the fusion protein does not comprise a VH, VL, Fab, Fv, or an scFv derived from an antibody).

In further embodiments, the fusion protein of the invention may comprise one or more modifications for enabling the capture of the fusion protein, including when the protein is bound to immune cells expressing a receptor comprising an antigen binding domain for binding dysfunctional P2X₇ receptor (such as a chimeric antigen receptor (CAR) or modified TCR).

It will be appreciated that typically the antigen binding domain of the receptor will be one which is capable of binding or recognising the same epitope of dysfunctional P2X₇ receptor that is comprised in the fusion protein. Moreover, it is within the purview of the skilled person, having knowledge of the specific epitope and binding target of a given anti-dysfunctional P2X₇ receptor CAR, to be able to design a suitable fusion protein in accordance with the invention, for use in binding to the CAR.

The one or more modifications to the fusion protein may be selected from: a biotin moiety, fluorescein (FITC), a peptide tag (such as His, Myc, Flag and related tags) and a magnetic label. Preferably the modification is a biotin moiety or a magnetic moiety.

The magnetic moiety can be any commercially available magnetic moiety for use in separation or capture of proteins or cells. For example, the magnetic moiety may comprise iron oxide microbeads (up to 50 nm diameter) or macrobeads (1-5 μm diameter). Preferably the magnetic moiety comprises microbeads.

In the case of biotin or magnetic moieties, the moieties may be conjugated to the fusion protein using any method known to the skilled person. In one example, the moieties are conjugated to the fusion protein via one or more lysine residues of the protein and/or at the amino-termini of the protein.

In the case of peptide tags, nucleic acid sequence encoding the tags can be included in the nucleic acid construct encoding the fusion protein, such that when expressed, the fusion protein is expressed with the tag already linked to the protein. Typically, the tag will be located at either the N or C terminus of the fusion protein. Preferably the tag will be located on the fusion protein so as not to interfere with the binding of the peptide (eg dysfunctional P2X₇ receptor epitope moiety) by a receptor on an immune cell, comprising an antigen binding domain for binding to the peptide.

In a second aspect, the present invention also provides a method for obtaining a population of immune cells, which are enriched for cells which express an exogenous cell surface receptor that comprises an antigen binding domain and intracellular signalling domain. Preferably, the receptor expressed by the immune cells is a chimeric antigen receptor (CAR), or optionally, a modified T cell receptor (TCR). Optionally, the exogenous cell surface receptor comprises an antigen binding domain for binding to a tumour associated or tumour specific antigen on a cancer cell although it will be appreciated that the exogenous cell surface receptor may be a receptor for use in a “universal CAR” system.

In a preferred embodiment of the second aspect, there is provided a method for obtaining a population of immune cells or for enriching for a population of cells expressing a receptor that comprises an antigen binding domain for binding to a tumour associated or tumour specific antigen on a cancer cell, the method comprising:
(i) providing a population of immune cells, preferably immune effector cells, wherein the cells have been subjected to transduction with a nucleic acid encoding a receptor comprising an antigen binding domain for binding to a tumour associated or tumour specific antigen on a cancer cell;
(ii) contacting the population of cells with a polypeptide, wherein the polypeptide comprises an epitope which is recognised by the antigen binding domain of the receptor, and wherein the polypeptide comprises a moiety for enabling capture of the polypeptide;
   to thereby form a complex of the polypeptide and the cells;
(iii) isolating the complex from the population of cells,
   thereby obtaining a population of immune cells expressing a receptor having an antigen binding domain for binding to a tumour associated or tumour specific antigen on a cancer cell.

Further, there is provided a method for obtaining a population of immune cells or for enriching for a population of cells expressing a receptor that comprises an antigen binding domain for binding to a tumour associated or tumour specific antigen on a cancer cell, the method comprising:
(i) providing a mixed population of immune cells, preferably immune effector cells, wherein a subpopulation of the cells express receptor comprising an antigen binding domain for binding to a tumour associated or tumour specific antigen on a cancer cell;
(ii) contacting the mixed population of cells with a polypeptide, wherein the polypeptide comprises an epitope which is recognised by the antigen binding domain of the receptor, and wherein the polypeptide comprises a moiety for enabling capture of the polypeptide;
   to thereby form a complex of the polypeptide and cells;
(iii) isolating the complex from the population of cells,
   thereby obtaining a population of immune cells expressing a receptor having an antigen binding domain for binding to a tumour associated or tumour specific antigen on a cancer cell.

Preferably, the cells are immune cells which express a chimeric antigen receptor (CAR) for binding to a tumour associated or tumour specific antigen on a cancer cell. Accordingly, there is also provided a method for obtaining a population of immune cells expressing a chimeric antigen receptor (CAR) for binding to a tumour associated or tumour specific antigen on a cancer cell, the method comprising:
(i) providing a population of immune cells, preferably immune effector cells, wherein the population comprises cells which have been transduced with a nucleic acid encoding a chimeric antigen receptor (CAR) for binding to a tumour associated or tumour specific antigen on a cancer cell; or wherein the cells express a chimeric antigen receptor (CAR) for binding to a tumour associated or tumour specific antigen on a cancer cell;
(ii) contacting the population of cells with a polypeptide, wherein the polypeptide comprises an epitope which is recognised by the CAR, and wherein the polypeptide comprises a moiety for enabling capture of the polypeptide;
   to thereby form a complex of the polypeptide and the cells;
(iii) isolating the complex from the population,
   thereby obtaining a population of immune cells expressing a chimeric antigen receptor (CAR) for binding to a tumour associated or tumour specific antigen on a cancer cell.

Further in accordance with the second aspect, there is provided a method for enriching a population of immune cells expressing a chimeric antigen receptor comprising an antigen binding domain for binding to a tumour associated or tumour specific antigen on a cancer cell, the method comprising:
(i) providing a population of immune cells, preferably immune effector cells, wherein the cells have been subjected to transduction with a nucleic acid encoding a chimeric antigen receptor (CAR) for binding to a tumour associated or tumour specific antigen on a cancer cell;
(ii) contacting the population of cells with a polypeptide, wherein the polypeptide comprises an epitope which is recognised by the CAR, and wherein the polypeptide comprises a moiety for enabling capture of the polypeptide,
   to thereby form a complex of the polypeptide and the cells;
(iii) isolating the complex of cells from the mixed population,
   thereby enriching a population of immune cells expressing a chimeric antigen receptor (CAR) for binding to a tumour associated or tumour specific antigen on a cancer cell.

Examples of tumour associated or tumour specific antigens which are typically targeted by cellular immunotherapeutics such as CARs, include, but are not limited to: dysfunctional (nf)P2X₇ receptor, mesothelin, EGFR, GPC3, MUC1, HER2, GD2, CEA, EpCAM, LeY, PCSA, CD19, CD20, Clec9a, CD276, PD-L1 and PD-L2. Other examples of target antigens are further described herein.

It will be well within the purview of the skilled person to generate a polypeptide (or nucleic acid encoding the polypeptide as the case may be), comprising an epitope that is recognised by a CAR (or other receptor for binding to a tumour antigen). For example, having knowledge of the amino acid sequence which is recognised or bound by a given CAR, the skilled person will be able to design a fusion protein (preferably a monomeric Fc fusion protein or asymmetric heterodimeric Fc fusion protein as described herein), for binding to the CAR, and for use in the methods. For example, in the context of a CAR for binding to CD19, the polypeptide will comprise a similar epitope of the CD19 molecule that is recognised by the receptor, fused to an Fc region of an antibody, and preferably having an Fc region as defined herein in any of SEQ ID NOs: 159 or 162, or an Fc region which is not capable of forming a homodimer.

In a preferred embodiment of the second aspect, the present invention also provides a use of a fusion protein according to the first aspect, or polypeptide as further described herein, for obtaining a population of immune cells, which are enriched for cells which express a receptor that comprises an antigen binding domain for binding to dysfunctional P2X₇ receptor. Preferably, the receptor expressed by the immune cells is a chimeric antigen receptor (CAR), or optionally, a modified T cell receptor (TCR).

In a preferred embodiment of the second aspect, there is provided a method for obtaining a population of immune cells or for enriching for a population of cells expressing a receptor that comprises an antigen binding domain for binding to dysfunctional P2X₇ receptor, the method comprising:
(i) providing a population of immune cells, preferably immune effector cells, wherein the cells have been subjected to transduction with a nucleic acid encoding a receptor comprising an antigen binding domain for binding to dysfunctional P2X₇ receptor;
(ii) contacting the population of cells with a polypeptide, wherein the polypeptide comprises an epitope of dysfunctional P2X₇ receptor which is recognised by the antigen binding domain of the receptor, and wherein the polypeptide comprises a moiety for enabling capture of the polypeptide;
   to thereby form a complex of the polypeptide and the cells;
(iii) isolating the complex from the population of cells,
   thereby obtaining a population of immune cells expressing a receptor having an antigen binding domain for binding to dysfunctional P2X₇ receptor.

Further, there is provided a method for obtaining a population of immune cells or for enriching for a population of cells expressing a receptor that comprises an antigen binding domain for binding to dysfunctional P2X₇ receptor, the method comprising:
(i) providing a mixed population of immune cells, preferably immune effector cells, wherein a subpopulation of the cells express receptor comprising an antigen binding domain for binding to dysfunctional P2X₇ receptor;
(ii) contacting the mixed population of cells with a polypeptide, wherein the polypeptide comprises an epitope of dysfunctional P2X₇ receptor which is recognised by the antigen binding domain of the receptor, and wherein the polypeptide comprises a moiety for enabling capture of the polypeptide;
   to thereby form a complex of the polypeptide and cells;
(iii) isolating the complex from the population of cells,
   thereby obtaining a population of immune cells expressing a receptor having an antigen binding domain for binding to dysfunctional P2X₇ receptor.

Preferably, the cells are immune cells which express a chimeric antigen receptor (CAR) for binding to a linear epitope of a P2X₇ receptor (such as an epitope of dysfunctional P2X₇ receptor) wherein the epitope comprises or consists of the amino acid sequence of SEQ ID NO: 7 (preferably the amino acid sequence of SEQ ID NO: 14), or comprises or consists of the amino acid sequence of any of SEQ ID NOs: 7 to 69 or 122 or sequences at least 80%, at least 81%, at least 82%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical thereto, provided that the sequences comprise at least the sequence set forth in SEQ ID NO: 14 or 7 or 9.

Accordingly, there is also provided a method for obtaining a population of immune cells expressing a chimeric antigen receptor (CAR) for binding to a linear epitope of a P2X₇ receptor (such as an epitope of dysfunctional P2X₇ receptor), the method comprising:
(i) providing a population of immune cells, preferably immune effector cells, wherein the population comprises cells which have been transduced with a nucleic acid encoding a chimeric antigen receptor (CAR) for binding to linear epitope of a P2X₇ receptor (such as an epitope of dysfunctional P2X₇ receptor); or wherein the cells express a chimeric antigen receptor (CAR) for binding to linear epitope of a P2X₇ receptor (such as an epitope of dysfunctional P2X₇ receptor);
(ii) contacting the population of cells with a polypeptide, wherein the polypeptide comprises an epitope of linear epitope of a P2X₇ receptor (such as an epitope of dysfunctional P2X₇ receptor) which is recognised by the CAR, and wherein the polypeptide comprises a moiety for enabling capture of the polypeptide;
   to thereby form a complex of the polypeptide and the cells;
(iii) isolating the complex from the population,
   thereby obtaining a population of immune cells expressing a chimeric antigen receptor (CAR) for binding to linear epitope of a P2X₇ receptor (such as an epitope of dysfunctional P2X₇ receptor).

Further in accordance with the second aspect, there is provided a method for enriching a population of immune cells expressing a chimeric antigen receptor comprising an antigen binding domain for binding to dysfunctional P2X₇ receptor, the method comprising:
(i) providing a population of immune cells, preferably immune effector cells, wherein the cells have been subjected to transduction with a nucleic acid encoding a chimeric antigen receptor (CAR) for binding to dysfunctional P2X₇ receptor;
(ii) contacting the population of cells with a polypeptide, wherein the polypeptide comprises an epitope of dysfunctional P2X₇ receptor which is recognised by the CAR, and wherein the polypeptide comprises a moiety for enabling capture of the polypeptide,
   to thereby form a complex of the polypeptide and the cells;
(iii) isolating the complex of cells expressing a chimeric antigen receptor (CAR) for binding to dysfunctional P2X₇ receptor bound to fusion protein, from the mixed population,
   thereby enriching a population of immune cells expressing a chimeric antigen receptor (CAR) for binding to dysfunctional P2X₇ receptor.

The present invention also finds application in the methods for enriching for a population of immune cells for use in a universal CAR system. Accordingly, in a further embodiment of the second aspect, there is provided a method for obtaining a population of immune cells or for enriching for a population of cells expressing an exogenous cell surface receptor that comprises an antigen binding domain for binding to a peptide, wherein the peptide comprises or consists of the amino acid sequence of SEQ ID NO: 7 or 14 (or optionally the amino acid sequence of any of SEQ ID NOs: 2 to 69 and 122); the method comprising:
(i) providing a population of immune cells, preferably immune effector cells, wherein the cells have been subjected to transduction with a nucleic acid encoding a receptor comprising an antigen binding domain for binding to the peptide;
(ii) contacting the population of cells with a polypeptide, wherein the polypeptide comprises the peptide that is recognised by the antigen binding domain of the receptor, and a moiety for enabling capture of the polypeptide;
   to thereby form a complex of the polypeptide and the cells;
(iii) isolating the complex from the population of cells,
   thereby obtaining a population of immune cells expressing a receptor having an antigen binding domain for binding to a peptide comprising or consisting of the amino acid sequence of SEQ ID NO: 7 or 14 (or optionally the amino acid sequence of any of SEQ ID NOs: 2 to 69 and 122).

Further, there is provided a method for obtaining a population of immune cells or for enriching for a population of cells expressing an exogenous cell surface receptor that comprises an antigen binding domain for binding to a peptide, wherein the peptide comprises or consists of the amino acid sequence of SEQ ID NO: 7 or 14 (or optionally the amino acid sequence of any of SEQ ID NOs: 2 to 69 and 122); the method comprising:
(i) providing a mixed population of immune cells, preferably immune effector cells, wherein a subpopulation of the cells express receptor comprising an antigen binding domain for binding to the peptide;
(ii) contacting the mixed population of cells with a polypeptide, wherein the polypeptide comprises the peptide that is recognised by the antigen binding domain of the receptor, and wherein the polypeptide comprises a moiety for enabling capture of the polypeptide;
   to thereby form a complex of the polypeptide and cells;
(iii) isolating the complex from the population of cells,
   thereby obtaining a population of immune cells expressing a receptor having an antigen binding domain for binding to a peptide comprising or consisting of the amino acid sequence of SEQ ID NO: 7 or 14 (or optionally the amino acid sequence of any of SEQ ID NOs: 2 to 69 and 122).

Preferably, the cells are immune cells which express a chimeric antigen receptor (CAR) for binding to the peptide. Accordingly, there is also provided a method for obtaining a population of immune cells expressing a chimeric antigen receptor (CAR) for binding to a peptide comprising or consisting of the amino acid sequence of SEQ ID NO: 7 or 14 (or optionally the amino acid sequence of any of SEQ ID NOs: 2 to 69 and 122), the method comprising:
(i) providing a population of immune cells, preferably immune effector cells, wherein the population comprises cells which have been transduced with a nucleic acid encoding a chimeric antigen receptor (CAR) for binding to a peptide comprising or consisting of the amino acid sequence of SEQ ID NO: 7 or 14 (or optionally the amino acid sequence of any of SEQ ID NOs: 2 to 69 and 122); or wherein the cells express a chimeric antigen receptor (CAR) for binding to a peptide comprising or consisting of the amino acid sequence of SEQ ID NO: 7 or 14 (or optionally the amino acid sequence of any of SEQ ID NOs: 2 to 69 and 122),
(ii) contacting the population of cells with a polypeptide, wherein the polypeptide comprises the peptide, and wherein the polypeptide comprises a moiety for enabling capture of the polypeptide;
   to thereby form a complex of the polypeptide and the cells;
(iii) isolating the complex from the population,
   thereby obtaining a population of immune cells expressing a chimeric antigen receptor (CAR) for binding to a peptide comprising or consisting of the amino acid sequence of SEQ ID NO: 7 or 14 (or optionally the amino acid sequence of any of SEQ ID NOs: 2 to 69 and 122).

Further in accordance with the second aspect, there is provided a method for enriching a population of immune cells expressing a chimeric antigen receptor comprising an antigen binding domain for binding to a peptide comprising or consisting of the amino acid sequence of SEQ ID NO: 7 or 14 (or optionally the amino acid sequence of any of SEQ ID NOs: 2 to 69 and 122), the method comprising:
(i) providing a population of immune cells, preferably immune effector cells, wherein the cells have been transduced with a nucleic acid encoding a chimeric antigen receptor (CAR) for binding to a peptide comprising or consisting of the amino acid sequence of SEQ ID NO: 7 or 14 (or optionally the amino acid sequence of any of SEQ ID NOs: 2 to 69 and 122),
(ii) contacting the population of cells with a polypeptide, wherein the polypeptide comprises the peptide, and wherein the polypeptide comprises a moiety for enabling capture of the polypeptide,
   to thereby form a complex of the polypeptide and the cells;
(iii) isolating the complex of cells expressing a chimeric antigen receptor (CAR) for binding to a peptide comprising or consisting of the amino acid sequence of SEQ ID NO: 7 or 14 (or optionally the amino acid sequence of any of SEQ ID NOs: 2 to 69 and 122) bound to polypeptide, from the mixed population,
   thereby enriching a population of immune cells expressing a chimeric antigen receptor (CAR) for binding to a peptide comprising or consisting of the amino acid sequence of SEQ ID NO: 7 or 14 (or optionally the amino acid sequence of any of SEQ ID NOs: 2 to 69 and 122).

In any embodiment of the second aspect of the invention, the polypeptide comprises the amino acid sequence of a fusion protein of any embodiment of the first aspect of the invention (such that the polypeptide comprises a peptide as described herein, such as an epitope of dysfunctional P2X₇ receptor, joined to an Fc region of an antibody).

In any embodiment of the second aspect of the invention, the polypeptide comprises a first portion comprising a peptide as described herein, such as an epitope of dysfunctional P2X₇ receptor, joined to a further amino acid sequence for facilitating the solubility and stability of the first portion. The further amino acid sequence joined to the peptide (eg dysfunctional P2X₇ receptor epitope) may comprise any suitable linker or hinge region, such as those exemplified in Tables 1 and 3. Such linker or hinge regions may comprise amino acid sequences comprised of glycine and serine repeats (so-called “GS” linker sequences, and variations thereof as further defined herein). The hinge region may also comprise sequences derived from the hinge region of an immunoglobulin, such as those defined in Table 3. Optionally, the linker sequence may comprise a cleavable sequence.

In further embodiments, the polypeptide may be in the form of a fusion protein comprising a peptide as described herein (such as an epitope of dysfunctional P2X₇ receptor) joined to a further amino acid sequence. The further sequence may comprise a serum albumin, transferrin, a carboxy-terminal peptide of chorionic gonadotropin (CG) p chain, a non-exact repeat peptide sequence, a polypeptide sequence composed of proline-alanine-serine polymer, an elastin-like peptide (ELP) repeat sequence), a homopolymer of glycine residues or a gelatin-like protein. The polypeptide may comprise a linker or hinge region, such as described above, forjoining the epitope of dysfunctional P2X₇ receptor to the further amino acid sequence.

Further still, the polypeptide may be in the form of a conjugate comprising a carbohydrate, a lipid, a liposome, a peptide, and an aptamer conjugated to the amino acid sequence comprising the peptide (eg epitope of dysfunctional P2X₇ receptor).

In any embodiment of the second aspect of the invention, the fusion protein or polypeptide may comprise of the amino acid sequence as set forth in any one of SEQ ID NOs: 145 to 158 or 160 and 161, or a sequence at least 80%, 85%, 86%, 87%, 88%, 89%, 90%, 91%, 92%, 93%, 94%, 95%, 96%, 97%, 98%, or 99% identical thereto.

In accordance with the second aspect of the invention, the moiety for enabling capture of the polypeptide may be any suitable moiety that can be bound by a capture agent or system, such as a biotin label, fluorescein (FITC), a peptide tag (such as His, Myc, Flag and related tags) or a magnetic label. Preferably the modification is a biotin moiety or a magnetic moiety.

The magnetic moiety can be any commercially available magnetic moiety for use in separation or capture of proteins or cells. For example, the magnetic moiety may be magnetic micro- or macro-beads.

In the case of biotin or magnetic moieties, the moieties may be conjugated to the polypeptide using any method known to the skilled person. In one example, the moieties are conjugated to the polypeptide via one or more lysine residues of the protein and/or at the amino-termini of the polypeptide.

In the case of peptide tags, nucleic acid sequence encoding the tags can be included in the nucleic acid construct encoding the polypeptide, such that when expressed, the polypeptide is expressed with the tag already linked to the protein. Typically, the tag will be located at either the N or C terminus of the polypeptide. Preferably the tag will be located on the polypeptide so as not to interfere with the binding of the dysfunctional P2X₇ receptor epitope moiety by a receptor on an immune cell, comprising an antigen binding domain for binding to the epitope moiety.

Optionally, where the polypeptide is labelled with a biotin moiety, the method may further comprise the step of contacting the cells (after step ii), with an anti-biotin antigen binding protein, preferably wherein the anti-biotin antigen binding protein comprises one or more moieties for enabling capture of the complex. Optionally the one or more moieties for enabling capture of the complex comprises iron oxide particles (micro- or macrobeads).

Optionally, wherein the polypeptide comprises a magnetic label, the step of isolating may comprise i) applying a magnetic field to the population of cells; ii) removing or discarding the cells that are not attracted to the magnetic field, iii) removal of the magnetic field to thereby provide a population of immune cells expressing a chimeric antigen receptor (CAR) for binding to dysfunctional P2X₇ receptor.

Optionally the method may further comprise the steps of expanding the isolated immune cells.

In any embodiment of the second aspect of the invention, the method may further comprise a step of treating the complex so as to release the cells from being bound by the polypeptide. Such methods will be known to the skilled person and will depend on the nature of the polypeptide moiety for enabling capture. For example, in the case of a biotinylated polypeptide, provision of excess free biotin to the complex will facilitate dissociation of the polypeptide bound by the capture agent (eg anti-biotin beads or the like).

In any aspect of the invention, the fusion protein or polypeptide may comprise a cleavable linker joining the epitope bound by the receptor (eg an epitope of dysfunctional P2X₇ receptor) and Fc region and/or further sequence for enabling capture of the fusion protein or polypeptide. Cleavable linkers are well known in the art and are further described herein. In any embodiment of the second aspect of the invention, the method may further comprise subjecting the polypeptide to treatment with a protease or other agent for cleaving the cleavable linker and thereby releasing the Fc region or further amino acid sequence therefrom.

Optionally the method further comprises the step of administering the isolated or enriched cells to a subject requiring treatment with the immune cells, such as cancer.

In certain embodiments, and depending on the capture agent used, the complex of immune cells/polypeptide may be administered directly to the subject.

In any embodiment of the second aspect, the population of immune cells is a population of effector immune cells, such as T cells, NK cells or NKT cells. Optionally the T cells are derived from stem cells, optionally wherein the stem cells are induced pluripotent stem cells (iPSCs) or embryonic stem cells.

Preferably, the population of immune cells are derived from a subject requiring treatment for cancer. Alternatively, the immune cells may be obtained from an allogeneic donor who does not require treatment.

In particularly preferred embodiments of the second aspect of the invention, the exogenous cell surface receptor that comprises an antigen binding domain (eg the chimeric antigen receptor. CAR), comprises an antigen binding domain that comprises the CDR amino acid sequences of PEP2-2-1 described in PCT/AU2010/001070 (WO2011020155, or in any one of the corresponding U.S. Pat. Nos. 9,127,059, 9,688,771, or U.S. Pat. No. 10,053,508). More preferably, the antigen binding domain of the receptor (eg CAR) comprises or consists of the amino acid sequence of the PEP2-2-1 antigen binding protein as described in PCT/AU2010/001070 (WO2011020155 or in any one of the corresponding U.S. Pat. Nos. 9,127,059, 9,688,771, or U.S. Pat. No. 10,053,508), incorporated herein by reference.

The present invention also provides a composition comprising a population of immune cells expressing a chimeric antigen receptor (CAR) for binding to dysfunctional P2X₇ receptor, wherein the population of cells is obtained by a method described herein. Preferably, the composition comprises greater than 30%, 40%, 50%, 60%, 70%, 80%, 85%, 90%, 91%, 92%, 93%, 94%, 95%, 96%, 97%, 98 or 99% of cells that express a chimeric antigen receptor (CAR) for binding to tumour associated or tumour specific antigen (such as dysfunctional P2X₇ receptor).

In further embodiments, there is provided a kit for use in a method described herein, the kit comprising:
a fusion protein capable of being bound by a CAR for binding to tumour associated or tumour specific antigen (such as dysfunctional P2X₇ receptor);
optionally, one or more reagents for enabling isolation of the fusion protein and complexes thereof.

Optionally, the kit comprises written instructions for use in a method of the second aspect of the invention.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### Sequence Information

TABLE 1
exemplary sequences of dysfunctional P2X₇ receptor and receptor
epitope moieties

| Peptide/ | | SEQ ID |
|---|---|---|
| protein name | Sequence | NO: |
| Full length P2X₇ | MPACCSCSDVFQYETNKVTRIQSMNYGTIKWFFHVIIFSYVCFAL | 1 |
| receptor | VSDKLYQRKEPVISSVHTKVKGIAEVKEEIVENGVKKLVHSVFDT | |
| E200 and E300 | ADYTFPLQGNSFFVMTNFLKTEGQEQRLCPEYPTRRTLCSSDR | |
| shown in bold | GCKKGWMDPQSKGIQTGRCVVYEGNQKTCEVSAWCPIEAVEE | |
| and underline | APRPALLNSAENFTVLIKNNIDFPGHNYTTRNILPGLNITCTFHKT | |
| Pro 210 shown in | QNPQCPIFRLGDIFRETGDNFSDVAIQGGIMGIEIYWDCNLDRWF | |
| italics | HHCRPKYSFRRLDDKTTNVSLYPGYNFRYAKYYKENNVEKRTLI | |
| | KVFGIRFDILVFGTGGKFDIIQLVVYIGSTLSYFGLAAVFIDFLIDTY | |
| | SSNCCRSHIYPWCKCCQPCVVNEYYYRKKCESIVEPKPTLKYVS | |
| | FVDESHIRMVNQQLLGRSLQDVKGQEVPRPAMDFTDLSRLPLAL | |
| | HDTPPIPGQPEEIQLLRKEATPRSRDSPVWCQCGSCLPSQLPES | |
| | HRCLEELCCRKKPGACITTSELFRKLVLSRHVLQFLLLYQEPLLAL | |
| | DVDSTNSRLRHCAYRCYATWRFGSQDMADFAILPSCCRWRIRK | |
| | EFPKSEGQYSGFKSPY | |
| Exemplary E200 | GHNYTTRNILPGLNITC | 2 |
| epitope | | |
| Variant E200 | GHNYTTRNILPGLNIT | 3 |
| epitope (E200′) | | |
| E300 | KYYKENNVEKRTLIK | 4 |
| Variant E300 | KYYKENNVEKRTLIKVF | 5 |
| epitope (E300′) | | |
| Composite | GHNYTTRNILPGAGAKYYKENNVEK | 6 |
| E200/E300 | | |
| epitope | | |
| E200 epitope | GHNYTTRNILPGLNITS | 7 |
| Cys to Ser | | |
| modification | | |
| (“Core” E200 | | |
| sequence) | | |
| Extended E200 | GHNYTTRNILPGLNITSTFHK | 8 |
| Cys to Ser | | |
| modification | | |
| Extended E200′ | GHNYTTRNILPGLNITSTFHKT | 9 |
| Cys to Ser | | |
| modification | | |
| Extended E200″ | GHNYTTRNILPGLNITSTFHKTC | 10 |
| Cys to Ser | | |
| modification | | |
| Extended Pep17 | GHNYTTRNILPGLNITSTFHKTSGSGK | 11 |
| Pep17 | GHNYTTRNILPGLNITSTFHKTS | 12 |
| Extended E200′ | DFPGHNYTTRNILPGLNITSTFHKT | 122 |
| Cys to Ser | | |
| modification + N | | |
| term ext | | |
| Pep16 | DFPGHNYTTRNILPGC | 13 |
| Minimum E200 | NYTTRNILPGL | 14 |
| sequence | | |
| E200_G4S | GHNYTTRNILPGLNITSGGGGS | 15 |
| E200_2xG4S | GHNYTTRNILPGLNITSGGGGSGGGGS | 16 |
| E200_3xG4S | GHNYTTRNILPGLNITSGGGGSGGGGSGGGGS | 17 |
| E200_extended | GHNYTTRNILPGLNITSTFHKTGS | 18 |
| peptide 17v3 | | |
| (24 aa) | | |
| E200_extended | GHNYTTRNILPGLNITSTFHGS | 19 |
| peptide 17v4 | | |
| (22 aa) | | |
| E200_extended | GHNYTTRNILPGLNITSGS | 20 |
| peptide 17v5 | | |
| (19 aa) | | |
| E200_extended | DFPGHNYTTRNILPGLNITSGS | 21 |
| peptide 17v6 | | |
| (22 aa) | | |
| E200_extended | DFPGHNYTTRNILPGLNITSGGGGS | 22 |
| peptide 17v7 | | |
| (25 aa) | | |
| E200_extended | DFPGHNYTTRNILPGLNITSGGGGSGGGGS | 23 |
| peptide 17v8 | | |
| (30 aa) | | |
| E200_extended | DFPGHNYTTRNILPGLNITSGGGGSGGGGSGGGGS | 24 |
| peptide 17v9 | | |
| (35 aa) | | |
| E200_extended | DFPGHNYTTRNILPGLNITSTFHKTSGSGK | 25 |
| peptide 17v10 | | |
| (30 aa) | | |
| E200_extended | DFPGHNYTTRNILPGLNITSTFHKTSGSGKGS | 26 |
| peptide 17v11 | | |
| (32 aa) | | |
| E200_extended | DFPGHNYTTRNILPGLNITSTFHKTSGSGKGGGGS | 27 |
| peptide 17v12 | | |
| (35 aa) | | |
| E200_extended | DFPGHNYTTRNILPGLNITSTFHGGGGS | 28 |
| peptide 17v13 | | |
| (25 aa) | | |
| E200_extended | GHNYTTRNILPGLNITSTFHGGGGS | 29 |
| peptide 17v14 | | |
| (22 aa) | | |
| E200_extended | DFPGHNYTTRNILPGLNITSTFHKTGGGGS | 30 |
| peptide 17v15 | | |
| (30 aa) | | |
| E200_extended | GHNYTTRNILPGLNITSTFHKTGGGGS | 31 |
| peptide 17v16 | | |
| (27 aa) | | |
| E200 + IgG hinge | GHNYTTRNILPGLNITSEPKSSDKTHT | 32 |
| (E200 | | |
| underlined) | | |
| E200 + GS | GHNYTTRNILPGLNITSGSEPKSSDKTHT | 33 |
| linker_IgG hinge | | |
| E200 + G4S | GHNYTTRNILPGLNITSGGGGSEPKSSDKTHT | 34 |
| linker + IgG hinge | | |
| Extended E200 + | GHNYTTRNILPGLNITSTFHKTSGSGKEPKSSDKTHT | 35 |
| _IgG hinge | | |
| Extended E200 + | GHNYTTRNILPGLNITSTFHKTSGSGKGSEPKSSDKTHT | 36 |
| GS linker + _IgG | | |
| hinge | | |
| Extended E200 + | GHNYTTRNILPGLNITSTFHKTSGSGKGGGGSEPKSSDKTHT | 37 |
| G4S linker + IgG | | |
| hinge | | |
| E200 + IgG | GHNYTTRNILPGLNITSEPKSSDKTHTGS | 38 |
| hinge + GS linker | | |
| E200 + GS | GHNYTTRNILPGLNITSGSEPKSSDKTHTGS | 39 |
| linker + IgG | | |
| hinge + GS linker | | |
| E200 + G4Slinker | GHNYTTRNILPGLNITSGGGGSEPKSSDKTHTGS | 40 |
| + IgG hinge + GS | | |
| linker | | |
| Extended | GHNYTTRNILPGLNITSTFHKTSGSGKEPKSSDKTHTGS | 41 |
| E200 + _IgG | | |
| hinge + GSlinker | | |
| Extended | GHNYTTRNILPGLNITSTFHKTSGSGKGSEPKSSDKTHTGS | 42 |
| E200 + GS | | |
| linker + _IgG | | |
| hinge + GS linker | | |
| Extended | GHNYTTRNILPGLNITSTFHKTSGSGKGGGGSEPKSSDKTHTGS | 43 |
| E200 + G4S | | |
| linker + _IgG | | |
| hinge + GS linker | | |
| E200 + IgG | GHNYTTRNILPGLNITSEPKSSDKTHTGGGGS | 44 |
| hinge + G4S linker | | |
| E200 + GS | GHNYTTRNILPGLNITSGSEPKSSDKTHTGGGGS | 45 |
| linker + IgG | | |
| hinge + G4S linker | | |
| E200 + G4S | GHNYTTRNILPGLNITSGGGGSEPKSSDKTHTGGGGS | 46 |
| linker + IgG | | |
| hinge + G4S linker | | |
| Extended | GHNYTTRNILPGLNITSTFHKTSGSGKEPKSSDKTHTGGGGS | 47 |
| E200 + IgG | | |
| hinge + G4S linker | | |
| Extended | GHNYTTRNILPGLNITSTFHKTSGSGKGSEPKSSDKTHTGGGGS | 48 |
| E200 + GS | | |
| linker + IgG | | |
| hinge + G4S linker | | |
| Extended | GHNYTTRNILPGLNITSTFHKTSGSGKGGGGSEPKSSDKTHTGG | 49 |
| E200 + G4S | GGS | |
| linker + IgG | | |
| hinge + G4S linker | | |
| N-term extended | DFPGHNYTTRNILPGLNITSEPKSSDKTHT | 50 |
| E200 + IgG hinge | | |
| N-term extended | DFPGHNYTTRNILPGLNITSGSEPKSSDKTHT | 51 |
| E200 + GS | | |
| linker + IgG hinge | | |
| N-term extended | DFPGHNYTTRNILPGLNITSGGGGSEPKSSDKTHT | 52 |
| E200 + G4S | | |
| linker + IgG hinge | | |
| N and C term | DFPGHNYTTRNILPGLNITSTFHKTSGSGKEPKSSDKTHT | 53 |
| extended | | |
| E200 + IgG hinge | | |
| N and C term | DFPGHNYTTRNILPGLNITSTFHKTSGSGKGSEPKSSDKTHT | 54 |
| extended | | |
| E200 + GS | | |
| linker + IgG hinge | | |
| N and C term | DFPGHNYTTRNILPGLNITSTFHKTSGSGKGGGGSEPKSSDKTH | 55 |
| extended E200 + | T | |
| G4S linker + IgG | | |
| hinge | | |
| N-term extended | DFPGHNYTTRNILPGLNITSEPKSSDKTHTGS | 56 |
| E200 + IgG hinge + | | |
| GS linker | | |
| N-term extended | DFPGHNYTTRNILPGLNITSGSEPKSSDKTHTGS | 57 |
| E200 + GS linker + | | |
| IgG hinge + | | |
| GS linker | | |
| N-term extended | DFPGHNYTTRNILPGLNITSGGGGSEPKSSDKTHTGS | 58 |
| E200 + G4S linker + | | |
| IgG hinge + | | |
| GS linker | | |
| N-term and C | DFPGHNYTTRNILPGLNITSTFHKTSGSGKEPKSSDKTHTGS | 59 |
| term extended | | |
| E200 + IgG | | |
| hinge + GS linker | | |
| N-term and C | DFPGHNYTTRNILPGLNITSTFHKTSGSGKGSEPKSSDKTHTGS | 60 |
| term extended | | |
| E200 + GS | | |
| linker + IgG | | |
| hinge + GS linker | | |
| N-term and C | DFPGHNYTTRNILPGLNITSTFHKTSGSGKGGGGSEPKSSDKTH | 61 |
| term extended | TGS | |
| E200 + G4S | | |
| linker + IgG | | |
| hinge + GS linker | | |
| N-term extended | DFPGHNYTTRNILPGLNITSEPKSSDKTHTGGGGS | 62 |
| E200 + IgG hinge + | | |
| G4S linker | | |
| N-term extended | DFPGHNYTTRNILPGLNITSGSEPKSSDKTHTGGGGS | 63 |
| E200 + GS linker + | | |
| IgG hinge + | | |
| G4S linker | | |
| N-term extended | DFPGHNYTTRNILPGLNITSGGGGSEPKSSDKTHTGGGGS | 64 |
| E200 + G4S linker + | | |
| IgG hinge + | | |
| G4S linker | | |
| N-term and C | DFPGHNYTTRNILPGLNITSTFHKTSGSGKEPKSSDKTHTGGGG | 65 |
| term extended | S | |
| E200 + IgG | | |
| hinge + G4S linker | | |
| N-term and C | DFPGHNYTTRNILPGLNITSTFHKTSGSGKGSEPKSSDKTHTGG | 66 |
| term extended | GGS | |
| E200 + GS | | |
| linker + IgG | | |
| hinge + G4S linker | | |
| N-term and C | DFPGHNYTTRNILPGLNITSTFHKTSGSGKGGGGSEPKSSDKTH | 67 |
| term extended | TGGGGS | |
| E200 + G4S | | |
| linker + IgG | | |
| hinge + G4S linker | | |
| IgG hinge + G4S | DKTHTSPPSPAPELLGGGGSDFPGHNYTTRNILPGLNITS | 68 |
| linker + N-term | | |
| extended E200 | | |
| (underlined) | | |
| G4S linker + IgG | GGGGSEPKSSDKTHTSPPSPAPELLGGGGSDFPGHNYTTRNILP | 69 |
| hinge + N-term | GLNITS | |
| extended E200 | | |
| (underlined) | | |
| G4S linker | GGGGS | 70 |
| IgG hinge region | EPKSSDKTHT | 71 |
| linker | | |
| GS linker + IgG | GSEPKSSDKTHTSPPSPAPELL | 72 |
| hinge | | |
| G4S linker + IgG | GGGGSEPKSSDKTHTSPPSPAPELLGGGGS | 73 |
| hinge+G4S | | |
| IgG hinge + GS | EPKSSDKTHTGS | 74 |
| linker | | |
| lgG hinge + G4S | EPKSSDKTHTGGGGS | 75 |
| linker | | |
| Exemplary C | TFHKT | 138 |
| terminal | | |
| extension of | | |
| E200 epitope | | |
| Exemplary N | DFP | 139 |
| terminal | | |
| extension of | | |
| E200 epitope | | |
| N terminal | DFPGHNYTTRNILPGLNITS | 140 |
| extended core E | | |
| 200 epitope | | |
| Exemplary IgG1 | EPKSCDKTHTSPPSPAP | 141 |
| hinge region for | | |
| monomeric | | |
| fusion proteins | | |
| (two C to S | | |
| mutations) | | |
| Exemplary IgG1 | EPKSSDKTHTSPPSPAP | 142 |
| hinge region for | | |
| monomeric | | |
| fusion proteins | | |
| (three C to S | | |
| mutations) | | |
| Exemplary G4S | GGGGS | 143 |
| linker sequence | | |
| Exemplary | LEVLFQGPVRR | 144 |
| cleavable linker | | |
| (protease | | |
| recognition site; | | |
| cleavage | | |
| between Q and | | |
| G residues) | | |
| Exemplary 200- | DFPGHNYTTRNILPGLNITSTFHKTSGSGKGGGGSEPKSCDKTH | 145 |
| Fc fusion | TSPPSPAPPVAGPSVFLFPPKPKDTLMISRTPEVTCVVVGVSHED | |
| sequence | PEVKFNWYVDGVEVHNAKTKPREEQYQSTYRVVSVLTVLHQDW | |
| ″DetR1″ | LNGKEYKCKVSNKQLPSPIEKTISKAKGQPREPQVYTLPPSRDEL | |
| (monomeric and | TKNQVSLTCLVKGFYPSDIAVEWESNGQPENNYKTTPPVLDSDG | |
| Fc attenuated: | SFFLYSKLTVDKSRWQQGNVFSCSVMHEALHNHYTQKSLSLSP | |
| E233P; | GK | |
| L234V;L235A; | | |
| ΔG236, D265G, | | |
| N297Q, A327Q, | | |
| A330S shown in | | |
| underline and | | |
| italics) | | |
| (E200 moiety | | |
| underlined; linker | | |
| in bold) | | |
| Exemplary 200- | GHNYTTRNILPGLNITSTFHKTSGSGKGGGGSEPKSCDKTHTSP | 146 |
| Fc fusion | PSPAPPVAGPSVFLFPPKPKDTLMISRTPEVTCVVVGVSHEDPE | |
| sequence | VKFNWYVDGVEVHNAKTKPREEQYQSTYRVVSVLTVLHQDWLN | |
| ″DetR2″ | GKEYKCKVSNKQLPSPIEKTISKAKGQPREPQVYTLPPSRDELTK | |
| (monomeric and | NQVSLTCLVKGFYPSDIAVEWESNGQPENNYKTTPPVLDSDGSF | |
| Fc attenuated | FLYSKLTVDKSRWQQGNVFSCSVMHEALHNHYTQKSLSLSPGK | |
| E233P; | | |
| L234V; L235A; | | |
| ΔG236, D265G, | | |
| N297Q, A327Q, | | |
| A330S) | | |
| (E200 moiety | | |
| underlined; linker | | |
| in bold; cys to | | |
| serine in italics) | | |
| Exemplary E200- | DFPGHNYTTRNILPGLNITSTFHKTSGSGKGGGGSEPKSSDKTH | 147 |
| Fc fusion | TSPPSPAPEAARGPSVFLFPPKPKDTLMISRTPEVTCVVVDVSHE | |
| sequence | DPEVKFNWYVDGVEVHNAKTKPREEQYNSTYRVVSVLTVLHQD | |
| ″DetR1″ | WLNGKEYKCKVSNKALPAPIEKTISKAKGQPREPQVYTLPPSRD | |
| +LALA + G236R | ELTKNQVSLTCLVKGFYPSDIAVEWESNGQPENNYKTTPPVLDS | |
| (monomeric and | DGSFFLYSKLTVDKSRWQQGNVFSCSVMHEALHNHYTQKSLSL | |
| Fc attenuated) | SPGK | |
| (E200 moiety | | |
| underlined; linker | | |
| in bold; cys to | | |
| serine in italics; | | |
| LALA + C1q | | |
| mutations in | | |
| italics and | | |
| underline) | | |
| Exemplary E200- | GHNYTTRNILPGLNITSTFHKTSGSGKGGGGSEPKSSDKTHTSP | 148 |
| Fc fusion | PSPAPEAARGPSVFLFPPKPKDTLMISRTPEVTCVVVDVSHEDP | |
| sequence | EVKFNWYVDGVEVHNAKTKPREEQYNSTYRVVSVLTVLHQDWL | |
| ″DetR2″ | NGKEYKCKVSNKALPAPIEKTISKAKGQPREPQVYTLPPSRDELT | |
| +LALA + G236R | KNQVSLTCLVKGFYPSDIAVEWESNGQPENNYKTTPPVLDSDGS | |
| (monomeric and | FFLYSKLTVDKSRWQQGNVFSCSVMHEALHNHYTQKSLSLSPG | |
| Fc attenuated) | K | |
| (E200 moiety | | |
| underlined; linker | | |
| in bold; cys to | | |
| serine in italics; | | |
| LALA + C1q | | |
| mutations in | | |
| italics and | | |
| underline) | | |
| Exemplary E200- | DFPGHNYTTRNILPGLNITSTFHKTSGSGKGGGGSEPKSCDKTH | 149 |
| Fc fusion | TCPPCPAPPVAGPSVFLFPPKPKDTLMISRTPEVTCVVVGVSHE | |
| sequence | DPEVKFNWYVDGVEVHNAKTKPREEQYQSTYRVVSVLTVLHQD | |
| ″DetR1″ | WLNGKEYKCKVSNKQLPSPIEKTISKAKGQPREPQVYTLPPSRD | |
| (dimeric Fc | ELTKNQVSLTCLVKGFYPSDIAVEWESNGQPENNYKTTPPVLDS | |
| attenuated) | DGSFFLYSKLTVDKSRWQQGNVFSCSVMHEALHNHYTQKSLSL | |
| (E200 moiety | SPGK | |
| underlined; linker | | |
| in bold) | | |
| Exemplary E200- | GHNYTTRNILPGLNITSTFHKTSGSGKGGGGSEPKSCDKTHTCP | 150 |
| Fc fusion | PCPAPPVAGPSVFLFPPKPKDTLMISRTPEVTCVVVGVSHEDPE | |
| sequence | VKFNWYVDGVEVHNAKTKPREEQYQSTYRVVSVLTVLHQDWLN | |
| ″DetR2″ | GKEYKCKVSNKQLPSPIEKTISKAKGQPREPQVYTLPPSRDELTK | |
| (dimeric Fc | NQVSLTCLVKGFYPSDIAVEWESNGQPENNYKTTPPVLDSDGSF | |
| attenuated) | FLYSKLTVDKSRWQQGNVFSCSVMHEALHNHYTQKSLSLSPGK | |
| (E200 moiety | | |
| underlined; linker | | |
| in bold) | | |
| Exemplary E200- | DFPGHNYTTRNILPGLNITSTFHKTSGSGKGGGGSEPKSSDKTH | 151 |
| Fc fusion | TCPPCPAPEAARGPSVFLFPPKPKDTLMISRTPEVTCVVVDVSH | |
| sequence | EDPEVKFNWYVDGVEVHNAKTKPREEQYNSTYRVVSVLTVLHQ | |
| ″DetR1″ | DWLNGKEYKCKVSNKALPAPIEKTISKAKGQPREPQVYTLPPSR | |
| +LALA + G236R | DELTKNQVSLTCLVKGFYPSDIAVEWESNGQPENNYKTTPPVLD | |
| (dimeric Fc | SDGSFFLYSKLTVDKSRWQQGNVFSCSVMHEALHNHYTQKSLS | |
| attenuated) | LSPGK | |
| (E200 moiety | | |
| underlined; linker | | |
| in bold; | | |
| LALA + C1q | | |
| mutations in | | |
| italics and | | |
| underline) | | |
| Exemplary E200- | GHNYTTRNILPGLNITSTFHKTSGSGKGGGGSEPKSSDKTHTCP | 152 |
| Fc fusion | PCPAPEAARGPSVFLFPPKPKDTLMISRTPEVTCVVVDVSHEDP | |
| sequence | EVKFNWYVDGVEVHNAKTKPREEQYNSTYRVVSVLTVLHQDWL | |
| ″DetR2″ | NGKEYKCKVSNKALPAPIEKTISKAKGQPREPQVYTLPPSRDELT | |
| +LALA + G236R | KNQVSLTCLVKGFYPSDIAVEWESNGQPENNYKTTPPVLDSDGS | |
| (dimeric Fc | FFLYSKLTVDKSRWQQGNVFSCSVMHEALHNHYTQKSLSLSPG | |
| attenuated) | K | |
| (E200 moiety | | |
| underlined; linker | | |
| in bold; | | |
| LALA + C1q | | |
| mutations in | | |
| italics and | | |
| underline) | | |
| Exemplary E200- | DFPGHNYTTRNILPGLNITSTFHKTSGSGKGGGGSLEVLFQGPV | 153 |
| Fc fusion | RREPKSSDKTHTSPPSPAPPVAGPSVFLFPPKPKDTLMISRTPEV | |
| sequence | TCVVVGVSHEDPEVKFNWYVDGVEVHNAKTKPREEQYQSTYRV | |
| ″DetR1″ | VSVLTVLHQDWLNGKEYKCKVSNKQLPSPIEKTISKAKGQPREP | |
| With cleavable | QVYTLPPSRDELTKNQVSLTCLVKGFYPSDIAVEWESNGQPENN | |
| sequence | YKTTPPVLDSDGSFFLYSKLTVDKSRWQQGNVFSCSVMHEALH | |
| between Fc | NHYTQKSLSLSPGK | |
| region and E200 | | |
| moiety | | |
| (monomeric Fc | | |
| attenuated) | | |
| (E200 moiety | | |
| underlined; linker | | |
| in bold; cleavable | | |
| linker in italics; | | |
| cys to serine | | |
| subs in italics) | | |
| Exemplary E200- | DFPGHNYTTRNILPGLNITSTFHKTSGSGKGGGGSLEVLFQGPV | 154 |
| Fc fusion | RREPKSSDKTHTCPPCPAPPVAGPSVFLFPPKPKDTLMISRTPE | |
| sequence | VTCVVVGVSHEDPEVKFNWYVDGVEVHNAKTKPREEQYQSTYR | |
| ″DetR1″ | VVSVLTVLHQDWLNGKEYKCKVSNKQLPSPIEKTISKAKGQPRE | |
| With cleavable | PQVYTLPPSRDELTKNQVSLTCLVKGFYPSDIAVEWESNGQPEN | |
| sequence | NYKTTPPVLDSDGSFFLYSKLTVDKSRWQQGNVFSCSVMHEAL | |
| between Fc | HNHYTQKSLSLSPGK | |
| region and E200 | | |
| moiety | | |
| (dimeric Fc | | |
| attenuated) | | |
| (E200 moiety | | |
| underlined; linker | | |
| in bold; cleavable | | |
| linker in italics) | | |
| Exemplary E200- | DFPGHNYTTRNILPGLNITSTFHKTSGSGKGGGGSEPKSSDKTH | 155 |
| Fc fusion | TSPPSPAPEAARGPSVFLFPPKPKDTLMASRTPEVTCVVVDVSH | |
| sequence | EDPEVKFNWYVDGVEVHNAKTKPREEQYNSTYRVVSVLTVLAQ | |
| ″DetR1″ | DWLNGKEYKCKVSNKALPAPIEKTISKAKGQPREPQVYTLPPSR | |
| +LALA + G236R: | DELTKNQVSLTCLVKGFYPSDIAVEWESNGQPENNYKTTPPVLD | |
| I253A/H310A | SDGSFFLYSKLTVDKSRWQQGNVFSCSVMHEALHNHYTQKSLS | |
| (monomeric, Fc | LSPGK | |
| attenuated and | | |
| FcRN | | |
| attenuated) | | |
| (E200 moiety | | |
| underlined; linker | | |
| in bold; | | |
| LALA + C1q | | |
| mutations in | | |
| italics and | | |
| underline; | | |
| cysteine | | |
| substitutions in | | |
| italics; | | |
| substitutions for | | |
| reducing FcRN | | |
| binding in bold | | |
| and underline) | | |
| Exemplary E200- | DFPGHNYTTRNILPGLNITSTFHKTSGSGKGGGGSEPKSSDKTH | 156 |
| Fc fusion | TCPPCPAPEAARGPSVFLFPPKPKDTLMASRTPEVTCVVVDVSH | |
| sequence | EDPEVKFNWYVDGVEVHNAKTKPREEQYNSTYRVVSVLTVLAQ | |
| ″DetR1″ | DWLNGKEYKCKVSNKALPAPIEKTISKAKGQPREPQVYTLPPSR | |
| +LALA + G236R: | DELTKNQVSLTCLVKGFYPSDIAVEWESNGQPENNYKTTPPVLD | |
| I253A/H310A | SDGSFFLYSKLTVDKSRWQQGNVFSCSVMHEALHNHYTQKSLS | |
| (dimeric, Fc | LSPGK | |
| attenuated and | | |
| FcRN | | |
| attenuated) | | |
| (E200 moiety | | |
| underlined; linker | | |
| in bold; | | |
| LALA+C1q | | |
| mutations in | | |
| italics and | | |
| underline ;; | | |
| substitutions for | | |
| reducing FcRN | | |
| binding in bold | | |
| and underline) | | |
| Exemplary E200- | DFPGHNYTTRNILPGLNITSTFHKTSGSGKGGGGSEPKSSDKTH | 157 |
| Fc fusion | TCPPCPAPPVAGPSVFLFPPKPKDTLMISRTPEVTCVVVGVSHE | |
| sequence | DPEVKFNWYVDGVEVHNAKTKPREEQYQSTYRVVSVLTVLHQD | |
| ″DetR1″ | WLNGKEYKCKVSNKQLPSPIEKTISKAKGQPREPQVYTLPPSRD | |
| (Fc attenuated) | ELTKNQVSLTCLVKGFYPSDIAVEWESNGQPENNYKTTPPVLDS | |
| (E200 moiety | DGSFFLYSKLTVDKSRWQQGNVFSCSVMHEALHNHYTQKSLSL | |
| underlined; linker | SPGK | |
| in bold) | | |
| ″Knob″ Fc region | | |
| Exemplary E200- | DFPGHNYTTRNILPGLNITSTFHKTSGSGKGGGGSEPKSSDKTH | 158 |
| Fc fusion | TSPPSPAPEAAGGPSVFLFPPKPKDTLMISRTPEVTCVVVDVSHE | |
| sequence | DPEVKFNWYVDGVEVHNAKTKPREEQYNSTYRVVSVLTVLHQD | |
| ″DetR1″ | WLNGKEYKCKVSNKALGAPIEKTISKAKGQPREPQVYTLPPSRK | |
| (Fc attenuated) | ELTKNQVSLTCLVKGFYPSDIAVEWESNGQPENNYKTTPPVLKS | |
| (E200 moiety | DGSFFLYSKLTVDKSRWQQGNVFSCSVMHEALHNHYTQKSLSL | |
| underlined; linker | SPGK | |
| in bold) | | |
| ″Knob″ Fc region | | |
| (no dimerisation | | |
| due to C to S | | |
| substitutions in | | |
| hinge region | | |
| ″Hole″ Fc region | EPKSSDKTHTCPPCPAPEAAGGPSVFLFPPKPKDTLMISRTPEVT | 159 |
| (no E200 moiety) | CVVVDVSHEDPEVKFNWYVDGVEVHNAKTKPREEQYNSTYRVV | |
| | SVLTVLHQDWLNGKEYKCKVSNKALGAPIEKTISKAKGQPREPQ | |
| | VYTLPPSRDELTKNQVSLTCLVKGFYPSDIAVEWESNGQPENNY | |
| | DTTPPVLDSDGSFFLYSDLTVDKSRWQQGNVFSCSVMHEALHN | |
| | HYTQKSLSLSPGK | |
| Exemplary E200- | DFPGHNYTTRNILPGLNITSTFHKTSGSGKGGGGSEPKSSDKTH | 160 |
| Fc fusion | TCPPCPAPEAAGGPSVFLFPPKPKDTLMISRTPEVTCVVVDVSH | |
| sequence | EDPEVKFNWYVDGVEVHNAKTKPREEQYNSTYRVVSVLTVLHQ | |
| ″DetR1″ | DWLNGKEYKCKVSNKALGAPIEKTISKAKGQPREPQVYTLPPSR | |
| (Fc attenuated) | DELTKNQVSLTCLVKGFYPSDIAVEWESNGQPENNYDTTPPVLD | |
| (E200 moiety | SDGSFFLYSDLTVDKSRWQQGNVFSCSVMHEALHNHYTQKSLS | |
| underlined; linker | LSPGK | |
| in bold) | | |
| ″Hole″ Fc region | | |
| Exemplary E200- | DFPGHNYTTRNILPGLNITSTFHKTSGSGKGGGGSEPKSSDKTH | 161 |
| Fc fusion | TCPPCPAPEAAGGPSVFLFPPKPKDTLMISRTPEVTCVVVDVSH | |
| sequence | EDPEVKFNWYVDGVEVHNAKTKPREEQYNSTYRVVSVLTVLHQ | |
| ″DetR1″ | DWLNGKEYKCKVSNKALGAPIEKTISKAKGQPREPQVYTLPPSR | |
| (Fc attenuated) | DELTKNQVSLTCLVKGFYPSDIAVEWESNGQPENNYDTTPPVLD | |
| (E200 moiety | SDGSFFLYSDLTVDKSRWQQGNVFSCSVMHEALHNHYTQKSLS | |
| underlined; linker | LSPGK | |
| in bold) | | |
| ″Hole″ Fc region | | |
| (no dimerisation | | |
| due to C to S | | |
| substitutions in | | |
| hinge region | | |
| ″Knob″ Fc region | EPKSSDKTHTSPPSPAPEAAGGPSVFLFPPKPKDTLMISRTPEVT | 162 |
| (no E200 moiety) | CVVVDVSHEDPEVKFNWYVDGVEVHNAKTKPREEQYNSTYRVV | |
| | SVLTVLHQDWLNGKEYKCKVSNKALGAPIEKTISKAKGQPREPQ | |
| | VYTLPPSRKELTKNQVSLTCLVKGFYPSDIAVEWESNGQPENNY | |
| | KTTPPVLKSDGSFFLYSKLTVDKSRWQQGNVFSCSVMHEALHN | |
| | HYTQKSLSLSPGK | |

### BRIEF DESCRIPTION OF THE DRAWINGS

FIG. 1: Enrichment of Jurkat cells stabilising transduced with nucleic acids encoding a CAR for binding dysfunctional P2X₇ receptor. A. shows the proportion of transduced cells enriched using a biotinylated protein comprising an amino acid sequence of the E200 epitope. B. shows that the cells 48 hours after enrichment retain high levels of purity and that the cells are not negatively impacted by the enrichment procedure. Grey shading indicates pre-enrichment; red shading indicates post-enrichment.

FIG. 2: Enrichment of primary donor cells (Donor 12) transduced with nucleic acids encoding a CAR for binding dysfunctional P2X₇ receptor. Grey shading indicates pre-enrichment; red shading indicates post-enrichment.

FIG. 3: Enrichment of primary donor cells (Donor 12) transduced with nucleic acids encoding an alternative CAR for binding dysfunctional P2X₇ receptor. Grey shading indicates pre-enrichment; red shading indicates post-enrichment. Enrichment was using MACS Column (Miltenyi Bioscience).

FIG. 4: Enrichment of primary donor cells (Donor 12) transduced with nucleic acids encoding an alternative CAR for binding dysfunctional P2X₇ receptor. Grey shading indicates pre-enrichment; red shading indicates post-enrichment. Enrichment was using Magnet Stand, Stem Cell Technologies.

FIG. 5: EGFR staining for direct staining of CAR expressing cells following enrichment using either monomeric or dimeric Fc attenuated fusion proteins comprising nfP2X₇ receptor epitope moiety for binding by the CAR. A=dimeric fusion protein (SEQ ID NO: 149), B=monomeric fusion protein (SEQ ID NO: 145); C=monomeric fusion protein (SEQ ID NO:146).

FIG. 6: Viability and purity of CAR expressing cells using monomeric Fc fusion comprising nfP2X₇ receptor epitope moiety. A. Staining with dye 7AAD to determine viability of enriched cells. B. Indirect CAR detection using tEGFR staining (AF647 primary labelled anti-EGFR mAb cetuximab, measured in the APC channel). C. Direct staining of nfP2X₇ CAR using biotinylated DetR2 (SEQ ID NO: 146), followed by staining with anti-biotin antibody Vioblue (130-113-857 Biotin Antibody, VioBlue®, Miltenyi Biotech.

FIG. 7: Proportion of CD25+/CD69+ and PD-L1+ cells at 24 hours (A), 48 hours (B) and 72 hours (C) following enrichment with monomeric or dimeric fusion proteins (having the amino acid sequence of SEQ ID NOs: 145 and 149, respectively).

FIG. 8: Transduction efficiency (%) on day 2 post MACS sorting using monomeric or dimeric fusion proteins (having the amino acid sequence of SEQ ID NOs: 145 and 149, respectively). D50, D53 and D71=T cells from donors 50, 53 and 71, respectively.

FIG. 9: Cell count (normalised to maximum expected cell count) following enrichment using monomeric or dimeric fusion proteins (having the amino acid sequence of SEQ ID NOs: 145 and 149, respectively). Cell counts on days 1 and 2 following enrichment are shown for T cells from healthy donors 50 (D50) and 71 (D71).

FIG. 10: Viability of enriched CAR T cells 2 days following MACS sorting. Percentage of 7AAD negative cells is shown. D50, D53 and D71=T cells from donors 50, 53 and 71, respectively.

FIG. 11: Proportion of CD25+/CD69+ and PD-L1+ cells, 2 days following MACS enrichment with monomeric or dimeric fusion proteins (having the amino acid sequence of SEQ ID NOs: 145 and 149, respectively). Results are shown for T cells from healthy donor 50 (D50).

### DETAILED DESCRIPTION OF THE EMBODIMENTS

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognise many methods and materials similar or equivalent to those described herein, which could be used in the practice of the present invention. The present invention is in no way limited to the methods and materials described.

It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

The present invention provides fusion proteins, compositions and kits comprising the same, and uses of the same in methods for enriching for a population of immune cells, preferably cells that express an exogenous cell surface receptor that comprises an antigen binding domain and intracellular signalling domain.

Preferably the fusion proteins comprise (i) a linear peptide epitope moiety derived from P2X₇ receptor (eg comprising an amino acid sequence derived from SEQ ID NO: 7 or 14) and that is recognised or capable of being bound by an antigen recognition domain of a receptor expressed on an immune cell, and (ii) a an Fc region of an antibody and optionally iii) a moiety for enabling capture of the fusion protein. The epitope moiety allows for specific binding of the fusion protein to the target immune cells for enrichment and the Fc region (preferably when comprising a moiety for enabling capture), enables capture of a complex of the fusion protein and the immune cells.

The inventors have demonstrated utility of both homodimeric and monomeric proteins derived from the fusion proteins described herein, for use in obtaining a population of immune cells or enriching for a population of immune cells.

In especially preferred embodiments, the Fc fusion proteins are designed so as to comprise only a single copy of the linear epitope derived from the P2X₇ receptor. This can be accomplished, as further described herein, by introducing amino acid substitutions into the Fc region to prevent homodimerisation, or alternatively, using the well-known knob-into-holes technology for ensuring formation of an asymmetric heterodimeric molecule (eg comprising a E200 peptide-Fc fusion protein and an Fc region that does not comprise an E200 peptide). Such monomeric or asymmetric heterodimeric molecules have the advantage of reducing activation of the target immune cells and preventing unwanted exhaustion of the target immune cells during the enrichment process c (as further described herein in the examples). Without wishing to be bound by theory, the inventors believe that this is due to the reduced ability of the molecules to cross-link either two different CAR receptors on one cell or two different CAR receptors on two separate CAR expressing cells.

### Definitions

Unless defined otherwise, technical and scientific terms used herein have the same meaning as commonly understood by one of ordinary skill in the art to which this invention belongs.

For purposes of interpreting this specification, the following definitions will generally apply and whenever appropriate, terms used in the singular will also include the plural and vice versa.

As used herein, the term “and/or”, e.g., “X and/or Y” will be understood to mean either “X and Y” or “X or Y” and shall be taken to provide explicit support for both meanings or for either meaning.

The articles “a” and “an” are used herein to refer to one or to more than one (i.e. to at least one) of the grammatical object of the article. By way of example, “a dysfunctional P2X₇ receptor epitope moiety” means one dysfunctional P2X₇ receptor epitope moiety or more than one dysfunctional P2X₇ receptor epitope moiety.

As used herein, except where the context requires otherwise, the term “comprise” and variations of the term, such as “comprising”, “comprises” and “comprised”, are not intended to exclude further additives, components, integers or steps.

As used herein “tumour-associated antigen” refers to an antigen that is expressed by cancer cells (the term “tumour-antigen” may also be used to refer to same). Tumour antigens are proteins that are produced by tumour cells that elicit an immune response, particularly T-cell mediated immune responses. Tumour antigens are well known in the art and include, for example, a glioma-associated antigen, carcinoembryonic antigen (CEA), β-human chorionic gonadotropin, alpha fetoprotein (AFP), lectin-reactive AFP, thyroglobulin RAGE-1, MN-CAIX, human telomerase reverse transcriptase, RU1, RU2 (AS), intestinal carboxyl esterase, mut hsp70-2, M-CSF, hK4 prostase, prostate-specific antigen (PSA), PAP, NY-ESO-1, LAGE-1a, p53, P501S prostein, PSMA, Her2/neu, survivin and telomerase, prostate-carcinoma tumour antigen-1 (PCTA-1), MAGE, ELF2M, neutrophil elastase, ephrinB2, CD22, insulin growth factor (IGF)-I, IGF-II, IGF-I receptor and mesothelin,

In one embodiment, the tumour antigen comprises one or more antigenic cancer epitopes associated with a malignant tumour. Malignant tumours express a number of proteins that can serve as target antigens for an immune attack. These molecules include but are not limited to tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu/ErbB-2. Yet another group of target antigens are onco-foetal antigens such as carcinoembryonic antigen (CEA). In B-cell lymphoma the tumour-specific idiotype immunoglobulin constitutes a truly tumour-specific immunoglobulin antigen that is unique to the individual tumour. B-cell differentiation antigens such as CD 19, CD20 and CD37 are other candidates for target antigens in B-cell lymphoma. Some of these antigens (CEA, HER-2, CD19, CD20, idiotype) have been used as targets for passive immunotherapy with monoclonal antibodies with limited success. The type of tumour antigen referred to in the invention may also be a tumour-specific antigen (TSA). A TSA is unique to tumour cells and does not occur on other cells in the body. A tumour-associated antigen (TAA) is not unique to a tumour cell and instead is also expressed on a normal cell under conditions that fail to induce a state of immunologic tolerance to the antigen. The expression of the antigen on the tumour may occur under conditions that enable the immune system to respond to the antigen. TAAs may be antigens that are expressed on normal cells during foetal development when the immune system is immature and unable to respond or they may be antigens that are normally present at extremely low levels on normal cells but which are expressed at much higher levels on tumour cells. Those tumour-associated antigens of greatest clinical interest are differentially expressed compared to the corresponding non-tumour tissue and allow for a preferential recognition of tumour cells by specific T-cells or immunoglobulins.

Non-limiting examples of TSA or TAA antigens include the following: Differentiation antigens such as MART-1/MelanA (MART-1), gp 100 (Pmel 17), tyrosinase, TRP-1, TRP-2 and tumour-specific multilineage antigens such as MAGE-1, MAGE-3, BAGE, GAGE-1, GAGE-2, p15; overexpressed embryonic antigens such as CEA; overexpressed oncogenes and mutated tumour-suppressor genes such as p53, Ras, HER-2/neu; unique tumour antigens resulting from chromosomal translocations; such as BCR-ABL, E2A-PRL, H4-RET, 1GH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p 180erbB-3, c-met, nm-23H 1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA195, CA242, CA-50, CAM43, CD68\P 1, CO-029, FGF-5, G250, Ga733\EpCAM, HTgp-175, M344, MA-50, MG7-Ag, MOV 18, NB/70K, NY-CO-1, RCAS 1, SDCCAG16, TA-90\Mac-2 binding protein\cyclophilin C-associated protein, TAAL6, TAG72, TLP, and TPS. Particularly preferred examples of tumour antigens in accordance with the present invention include: CD33 (Siglec-3), CD123 (IL3RA), CD135 (FLT-3), CD44 (HCAM), CD44V6, CD47, CD184 (CXCR4), CLEC12A (CLL1), LeY, FRp, MICA/B, CD305 (LAIR-1), CD366 (TIM-3), CD96 (TACTILE), CD133, CD56, CD29 (ITGB1), CD44 (HCAM), CD47 (IAP), CD66 (CEA), CD112 (Nectin2), CD117 (c-Kit), CD133, CD146 (MCAM), CD155 (PVR), CD171 (LI CAM), CD221 (IGF1), CD227 (MUC1), CD243 (MRD1), CD246 (ALK), CD271 (LNGFR), CD19, CD20, GD2, and especially EGFR, mesothelin, GPC3, MUC1, HER2, GD2, CEA, EpCAM, LeY, PCSA CD276 and dysfunctional (nf)P2X₇ receptor.

“Purinergic receptor” generally refers to a receptor that uses a purine (such as ATP) as a ligand.

“P2X₇ receptor” generally refers to a purinergic receptor formed from three protein subunits or monomers, with at least one of the monomers having an amino acid sequence substantially as shown in SEQ ID NO: 1 in Table 1 herein.

To the extent that P2X₇ receptor is formed from three monomers, it is a “trimer” or “trimeric”. “P2X₇ receptor” encompasses naturally occurring variants of P2X₇ receptor, e.g., wherein the P2X₇ monomers are splice variants, allelic variants, SNPs and isoforms including naturally-occurring truncated or secreted forms of the monomers forming the P2X₇ receptor (e.g., a form consisting of the extracellular domain sequence or truncated form of it), naturally-occurring variant forms (e.g., alternatively spliced forms) and naturally-occurring allelic variants. In certain embodiments of the invention, the native sequence P2X₇ monomeric polypeptides disclosed herein are mature or full-length native sequence polypeptides comprising the full-length amino acids sequence shown in SEQ ID NO: 1. In certain embodiments the P2X₇ receptor may have an amino acid sequence that is modified, for example various of the amino acids in the sequence shown in SEQ ID NO: 1 may be substituted, deleted, or a residue may be inserted.

“Functional P2X₇ receptor” generally refers to a form of the P2X₇ receptor having three intact binding sites or clefts for binding to ATP. When bound to ATP, the functional receptor forms a non-selective sodium/calcium channel that converts to a pore-like structure that enables the ingress of calcium ions and molecules of up to 900 Da into the cytosol, one consequence of which may be induction of programmed cell death. In normal homeostasis, expression of functional P2X₇ receptors is generally limited to cells that undergo programmed cell death such as thymocytes, dendritic cells, lymphocytes, macrophages and monocytes. There may also be some expression of functional P2X₇ receptors on erythrocytes and other cell types.

“Dysfunctional P2X₇ receptor” (also called “non-functional” or (nf) P2X₇) is a P2X₇ receptor that has an impaired response to ATP such that it is unable to form an apoptotic pore under physiological conditions. A dysfunctional P2X₇ receptor or (nfP2X₇ receptor) generally refers to a form of a P2X₇ receptor having a conformation, distinct from functional P2X₇, whereby the receptor is unable to form an apoptotic pore, but which is still able to operate as a non-selective channel through the maintenance of a single functional ATP binding site located between adjacent monomers. One example arises where one or more of the monomers has a cis isomerisation at Pro210 (according to SEQ ID NO: 1). The isomerisation may arise from any molecular event that leads to misfolding of the monomer, including for example, mutation of monomer primary sequence or abnormal post translational processing. One consequence of the isomerisation is that the receptor is unable to bind to ATP at one, or more particularly two, ATP binding sites on the trimer and as a consequence not be able to extend the opening of the channel. In the circumstances, the receptor cannot form a pore and this limits the extent to which calcium ions may enter the cytosol. Dysfunctional P2X₇ receptors are expressed on a wide range of epithelial and haematopoietic cancers. As used herein, the term “dysfunctional P2X₇ receptors” may be used interchangeably with the term “non-functional P2X₇ receptors” or “nfP2X₇ receptors”.

“Cancer associated-P2X₇ receptors” are generally P2X₇ receptors that are found on cancer cells (including, pre-neoplastic, neoplastic, malignant, benign or metastatic cells), but not on non-cancer or normal cells.

“E200 epitope” generally refers to an epitope having the sequence GHNYTTRNILPGLNITC (SEQ ID NO: 2). Variants thereof are exemplified in Table 1 and include any of SEQ ID NOs: 3, or 7 to 69 and 122.

“E300 epitope” generally refers to an epitope having the sequence KYYKENNVEKRTLIK (SEQ ID NO: 4) or a variant thereof, as defined in SEQ ID NO: 5.

A “composite epitope” generally refers to an epitope that is formed from the juxtaposition of the E200 and E300 epitopes or parts of these epitopes. An example of a composite epitope comprising E200 and E300 epitopes is GHNYTTRNILPGAGAKYYKENNVEK (SEQ ID NO: 6).

As used herein, the term “antigen” is intended to include substances that bind to or evoke the production of one or more antibodies and may comprise, but is not limited to, proteins, peptides, polypeptides, oligopeptides, lipids, carbohydrates, and combinations thereof, for example a glycosylated protein or a glycolipid. The term “antigen” as used herein refers to a molecular entity that may be expressed on a target cell and that can be recognised by means of the adaptive immune system including but not restricted to antibodies or TCRs, or engineered molecules including but not restricted to transgenic TCRs, CARs, scFvs or multimers thereof, Fab-fragments or multimers thereof, antibodies or multimers thereof, single chain antibodies or multimers thereof, or any other molecule that can execute binding to a structure with high affinity.

“Epitope” generally refers to that part of an antigen that is bound by the antigen binding site of an antibody. An epitope may be “linear” in the sense that the hypervariable loops of the antibody CDRs that form the antigen binding site bind to a sequence of amino acids as in a primary protein structure. In certain embodiments, the epitope is a “conformational epitope” i.e. one in which the hypervariable loops of the CDRs bind to residues as they are presented in the tertiary or quaternary protein structure.

The terms “binds to”, “specifically binds to” or “specific for” with respect to a receptor referring to an antigen-binding domain that recognises and binds a dysfunctional P2X₇ receptor, is intended to mean that the receptor does not substantially recognise or bind to other antigens in a sample.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity, which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention.

The terms “immune cell” or “immune effector cell” refer to a cell that may be part of the immune system and executes a particular effector function such as alpha-beta T cells, NK cells, NKT cells, B cells, Breg cells, Treg cells, innate lymphoid cells (ILC), cytokine induced killer (CIK) cells, lymphokine activated killer (LAK) cells, gamma-delta T cells, mesenchymal stem cells or mesenchymal stromal cells (MSC), monocytes or macrophages or any hematopoietic progenitor cells such as pluripotent stem cells and early progenitor subsets that may mature or differentiate into somatic cells. The cells may be naturally occurring or generated by cytokine exposure, artificial/genetically modified cells (such as iPSCs and other artificial cell types). Preferred immune cells are cells with cytotoxic effector function such as alpha-beta T cells, NK cells, NKT cells, ILC, CIK cells, LAK cells or gamma-delta T cells. “Effector function” means a specialised function of a cell, e.g. in a T cell an effector function may be cytolytic activity or helper cell activity including the secretion of cytokines.

The term “autologous” as used herein refers to any material derived from the same subject to whom it is later re-introduced.

The term “allogeneic” as used herein refers to any material derived from a different subject of the same species as the subject to whom the material is re-introduced.

An “enriched” or “purified” population of cells is an increase in the ratio of particular cells to other cells, for example, in comparison to the cells as found in a subject's body, or in comparison to the ratio prior to exposure to a peptide, nucleic acid or vector of the invention. In some embodiments, in an enriched or purified population of cells, the particular cells include at least 20%, 30%, 40%, 50%, 60%, 70%, 75%, 80%, 90%, 95% or 99% of the total cell population. A population of cells may be defined by one or more cell surface markers and/or properties.

The terms “engineered cell” and “genetically modified cell” as used herein can be used interchangeably. The terms mean containing and/or expressing a foreign gene or nucleic acid sequence that in turn modifies the genotype or phenotype of the cell or its progeny. Especially, the terms refer to the fact that cells, preferentially immune cells, can be manipulated by recombinant methods well known in the art to express stably or transiently peptides or proteins that are not expressed in these cells in the natural state. For example, immune cells are engineered to express an artificial construct such as a chimeric antigen receptor on their cell surface. For example, the CAR sequences may be delivered into cells using an adenoviral, adeno-associated viral (AAV)-based, retroviral or lentiviral vector or any other pseudotyped variations thereof or any other gene delivery mechanism such as electroporation or lipofection with CRISPR/Cas9, transposons (e.g. sleeping-beauty) or variations thereof. The gene delivery may be in the form of mRNA (transient) or DNA (transient or permanent).

Amino acid structure and single and three letter abbreviations used throughout the specification are defined in Table 2, which lists the twenty proteinogenic naturally occurring amino acids which occur in proteins as L-isomers.

TABLE 2

| | |
|---|---|
| | (1) |
| 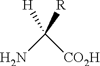 | |
| | (2) |
| 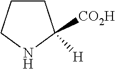 | |

| | Three-letter | One-letter | Structure of side chain |
|---|---|---|---|
| Amino Acid | Abbreviation | symbol | (R) |
| Alanine | Ala | A | —CH₃ |
| Arginine | Arg | R | —(CH₂)₃NHC(═N)NH₂ |
| Asparagine | Asn | N | —CH₂CONH₂ |
| Aspartic acid | Asp | D | —CH₂CO₂H |
| Cysteine | Cys | C | —CH₂SH |
| Glutamine | Gln | Q | —(CH₂)₂CONH₂ |
| Glutamic acid | Glu | E | —(CH₂)₂CO₂H |
| Glycine | Gly | G | —H |
| Histidine | His | H | —CH₂(4-imidazolyl) |
| Isoleucine | Ile | I | —CH(CH₃)CH₂CH₃ |
| Leucine | Leu | L | —CH₂CH(CH₃)₂ |
| Lysine | Lys | K | —(CH₂)₄NH₂ |
| Methionine | Met | M | —(CH₂)₂SCH₃ |
| Phenylalanine | Phe | F | —CH₂Ph |
| Proline | Pro | P | see formula (2) above for |
| | | | structure of amino acid |
| Serine | Ser | S | —CH₂OH |
| Threonine | Thr | T | —CH(CH₃)OH |
| Tryptophan | Trp | W | —CH₂(3-indolyl) |
| Tyrosine | Tyr | Y | —CH₂(4-hydroxyphenyl) |
| Valine | Val | V | —CH(CH₃)₂ |

As used herein, the term “non-proteinogenic amino acid” refers to an amino acid having a side chain that does not occur in the naturally occurring L-a-amino acids recited in Table 2. Examples of non-proteinogenic amino acids and derivatives include, but are not limited to, norleucine, 4-aminobutyric acid, 4-amino-3-hydroxy-5-phenylpentanoic acid, 6-aminohexanoic acid, t-butylglycine, norvaline, phenylglycine, ornithine, citrulline, sarcosine, 4-amino-3-hydroxy-6-methylheptanoic acid, 2-thienyl alanine and/or D-isomers of natural amino acids

As used herein, the term “a-amino acid” refers to an amino acid that has a single carbon atom (the a-carbon atom) separating a carboxyl terminus (C-terminus) and an amino terminus (N-terminus). An a-amino acid includes naturally occurring and non-naturally occurring L-amino acids and their D-isomers and derivatives thereof such as salts or derivatives where functional groups are protected by suitable protecting groups. Unless otherwise stated, the term “amino acid” as used herein refers to an a-amino acid.

The term “alkyl” refers to a straight chain or branched saturated hydrocarbon group having 1 to 6 carbon atoms. Where appropriate, the alkyl group may have a specified number of carbon atoms, for example, C₁₋₆alkyl which includes alkyl groups having 1, 2, 3, 4, 5 or 6 carbon atoms in a linear or branched arrangement. Examples of suitable alkyl groups include, but are not limited to, methyl, ethyl, n-propyl, i-propyl, n-butyl, i-butyl, t-butyl, n-pentyl, 2-methylbutyl, 3-methylbutyl, 4-methylbutyl, n-hexyl, 2-methylpentyl, 3-methylpentyl, 4-methylpentyl, and 5-methylpentyl.

As used herein, the term “subject” refers to a mammal such as mouse, rat, cow, pig, goat, chicken, dog, monkey or human. Preferentially, the subject is a human. The subject may be a subject suffering from a disorder such as cancer (a patient). As used herein, the terms “subject” and “individual” may be used interchangeably.

### Receptor Epitope Moiety (Dysfunctional P2X₇ Receptor Epitope Moiety)

The present invention relates to methods which utilise polypeptides which comprise an epitope that is recognised by the antigen binding domain of a receptor on an immune cell (such as a CAR or TCR). The receptors are typically for binding to tumour associated or tumour specific antigens on cancer cells, and consequently, the epitope moieties of the polypeptides for use according to the invention, comprise a sequence that is derived from the tumour associated or tumour specific antigen that is bound by the receptor.

In certain examples, the immune cell comprises a receptor for binding the extracellular domain of CD19 on a target cell. It will be appreciated that in such examples, the polypeptide for use in accordance with the second aspect of the invention will comprise the same epitope of the ECD of CD19 that is bound by the receptor. Cellular immunotherapeutics having receptor for targeting CD19 are known to the skilled person, as are the epitopes to which such immunotherapeutics bind. For example, for enriching for CAR-T cells comprising a CAR that has an antigen-recognition domain consisting of anti-CD19 scFv FMC683, the skilled person will appreciate that the polypeptide for use in accordance with the second aspect of the invention should comprise an epitope that is bound by scFv FMC683. Similarly, for obtaining or enriching for CAR-T cells comprising a CAR that has an antigen-recognition domain consisting of anti-CD19 scFv A3B1, the skilled person will appreciate that the polypeptide for use in accordance with the second aspect of the invention should comprise an epitope that is bound by scFv A3B1. The epitopes bound by anti-CD19 antibodies FMC683, 3B10, and 4G7-2E3, which are used in various anti-CD19 cellular immunotherapeutics, are described in Klesmith et al., (2019) Biochemistry, 58:489-4881, incorporated herein by reference.

In a similar example, the immune cell may comprise a receptor for binding CD20 on a target cell. It will be appreciated that in such examples, the polypeptide for use in accordance with the second aspect of the invention will comprise the same epitope of CD20 that is bound by the receptor.

In other examples, the immune cell may comprise a receptor (eg CAR) for binding mesothelin and therefore the polypeptide comprises an epitope of mesothelin.

In other examples, the immune cells may comprise a receptor (eg CAR) for binding EGFR and therefore the polypeptide comprises an epitope of EGFR.

In other examples, the immune cell may comprise a receptor (eg CAR) for binding GPC3 and therefore the polypeptide comprises an epitope of GPC3.

In other examples, the immune cell may comprise a receptor for binding MUC1 and therefore the polypeptide comprises an epitope of the MUC1.

In other examples, the immune cell comprises a receptor for binding HER2 and therefore the polypeptide comprises an epitope of HER2.

In other examples, the immune cell comprises a receptor for binding GD2 and therefore the polypeptide comprises an epitope of GD2.

In other examples, the immune cell comprises a receptor for binding CEA and therefore the polypeptide comprises an epitope of CEA.

In other examples, the immune cell comprises a receptor for binding EpCAM and therefore the polypeptide comprises an epitope of EpCAM.

In other examples, the immune cell comprises a receptor for binding LeY and therefore the polypeptide comprises an epitope of LeY.

In other examples, the immune cell comprises a receptor for binding PSCA and therefore the polypeptide comprises an epitope of PCSA.

In other examples, the immune cell comprises a receptor for binding CD276 and therefore the polypeptide comprises an epitope of CD276.

The present invention also provides fusion proteins comprising a dysfunctional P2X₇ receptor epitope moiety.

The dysfunctional P2X₇ receptor epitope moiety may be provided in the form of a dysfunctional P2X₇ receptor, or a fragment of a dysfunctional P2X₇ receptor, that has at least one of the three ATP binding sites that are formed at the interface between adjacent correctly packed monomers that are unable to bind ATP. Such receptors are unable to extend the opening of the non-selective calcium channels to apoptotic pores.

In accordance with the present invention, the dysfunctional P2X₇ receptor epitope moiety is typically in the form of a peptide fragment of a dysfunctional P2X₇ receptor. Typically, the peptide comprises an epitope that is not found or not available for binding on a functional P2X₇ receptor.

In some embodiments, the peptide comprises the proline at amino acid position 210 of the dysfunctional P2X₇ receptor. In some embodiments, the peptide comprises one or more amino acid residues spanning from glycine at amino acid position 200 to cysteine at amino acid position 216, inclusive, of the dysfunctional P2X₇ receptor.

A range of peptide fragments of a dysfunctional P2X₇ receptor are known and discussed in PCT/AU2002/000061 (and in corresponding publications WO 2002/057306 and U.S. Pat. Nos. 7,326,415, 7,888,473, 7,531,171, 8,080,635, 8,399,617, 8,709,425, 9,663,584, or U.S. Pat. No. 10,450,380), PCT/AU2008/001364 (and in corresponding publications WO 2009/033233 and U.S. Pat. Nos. 8,440,186, 9,181,320, 9,944,701 or US 10,597,45) and PCT/AU2009/000869 (and in corresponding publications WO 2010/000041 and U.S. Pat. No. 8,597,643, 9,328,155 or 10,238,716) the contents of all of which are incorporated in entirety. Exemplary peptides within these specifications which include epitopes contemplated for use in this invention are described below.

| PCT publication | Peptide sequence |
|---|---|
| WO 2002/057306 | GHNYTTRNILPGLNIT (SEQ ID NO: 3) |
| WO 2002/057306 | GHNYTTRNILPGLNITC (SEQ ID NO: 2) (also referred to |
| | herein as the “E200” epitope) |
| WO 2009/033233 | KYYKENNVEKRTLIKVF (SEQ ID NO: 4) (also referred to |
| | herein as the “E300” epitope) |
| WO 2010/000041 | GHNYTTRNILPGAGAKYYKENNVEK (SEQ ID NO: 6) (also |
| | referred to herein as the “E200/E300” or “composite” epitope) |

Non-limiting examples of variations of the E200 peptide sequence (including with N and/or C terminal extensions, and various linker, hinge or spacer regions) are provided in Table 1.

The amino acid sequences of any one of SEQ ID NOs: 2 to 69 or 155 may comprise a portion of the epitope moiety that is recognised or capable of being bound by a receptor expressed on an immune cell (also referred to herein as the “recognition sequence” of the epitope moiety).

In some embodiments, the epitope moiety comprises or consists of an amino acid sequence selected from any of the peptide sequences listed in Table 1 above.

In some embodiments, the N-terminus of the epitope moiety is a free amine (—NH₂).

In some embodiments, the C-terminus of the epitope moiety is a free acid (—COOH). In some embodiments, the C-terminus is a derivative or analogue of a free acid group, for example an ester (—COOC1-6alkyl) or a primary or secondary amide (—CONHR4 wherein R4 is selected from H and C1-6alkyl). Advantageously, having a C-terminus that is a derivative or analogue of a free acid group may improve the biological stability of the peptide compared to the free acid. In some embodiments, the C-terminus is a derivative or analogue of a free acid group that comprises a functional moiety, for example biotin.

In any embodiment of the first or second aspects, the epitope of a dysfunctional P2X₇ receptor, comprises or consists of an epitope that is only found on dysfunctional P2X₇ receptor but is not found on a functional form of the P2X₇ receptor. In other words, preferably, the polypeptide comprises or consists of an epitope that is specific to a dysfunctional P2X₇ receptor.

In further embodiments of the first or second aspects, the fusion protein comprises an epitope corresponding to the E200, E300 or composite E200/E300 epitopes as herein defined. It will be within the purview of the skilled person to obtain various polypeptides for use in accordance with the invention. For example the skilled person will appreciate that it is possible to include additional amino acids N- or C-terminal to the region comprising the epitope bound by the anti-nfP2X₇ receptor CAR. In a non-limiting example, and in the context of E200, which is typically defined as having an amino acid sequence substantially as defined in SEQ ID NO: 2 or 7 (and having a minimum sequence as defined in SEQ ID NO: 14), additional amino acids derived from the native sequence of P2X₇ receptor can be included in the polypeptide, for example, the residues “DFP” N-terminal to the epitope in the P2X₇ receptor sequence and/or residues “TFHKT” C-terminal to the epitope in the P2X₇ receptor sequence. In any embodiment, the polypeptide may comprise at least 1, at least 2, at least 3, at least 4, at least 5 or at least 6 amino acids derived from the P2X₇ receptor sequence, in addition to the sequence of the E200 or E300 or composite epitopes.

In a preferred embodiment, the sequence of the E200 epitope is further modified to substitute the cysteine residue (residue 17 in SEQ ID NO: 2) to a serine residue (eg to provide the sequence of SEQ ID NO: 7). The skilled person will appreciate that this can be done to reduce likelihood of any disulphide bonding between the polypeptide and another molecule.

It will also be within the purview of the skilled person to include additional amino acid residues to the E200, E300 or composite epitopes (or extended epitopes as discussed in the paragraph above), such as, for example, by the addition of at least 1, at least 2, at least 3, at least 4, at least 5, at least 6, at least 7, at least 8, at least 9 or at least 10 additional amino acid residues to the N- and C-terminal regions of peptides consisting of the amino acid sequence of the relevant epitope. Typically such additional amino acids can be derived from linker sequences (such as peptides comprising glycine and serine residues); or be derived from the hinge region of an immunoglobulin. Typically no more than 30, no more than 25, or no more than 20 amino acid residues are added to the N- and/or C-terminal residues of the E200, E300 or composite epitopes as defined herein. Examples of such extended E200 epitope peptides are provided in Table 1.

### Fc Region

In any embodiment, the amino acid sequence of the epitope of a dysfunctional P2X₇ receptor may be fused via its C terminal region to the N terminal region of an Fc region of an antibody, or variant thereof. In any embodiment, the amino acid sequence of epitope of a dysfunctional P2X₇ receptor may be fused via its N terminal region to the C terminal region of an Fc region of an antibody, or variant thereof.

Preferably, the Fc region of the fusion protein comprises two heavy chain fragments, more preferably the CH2 and CH3 domains of said heavy chain.

The Fc region may comprise one or more amino acid sequence modifications compared to naturally occurring Fc sequences. The Fc region may comprise one or more amino acid substitutions, such as substitution of one or more cysteine residues, so as to prevent dimerisation of the molecule to identical molecules. It will be appreciated that any amino acid substitution which prevents dimerisation of the Fc regions may be employed. As such, in vivo, the Fc fusion proteins described herein may be monomeric proteins. Preferably, the Fc region and hinge region derived from an immunoglobulin comprises substitution of at least one, at least two or at least three cysteine residues. Preferably the substituted residues are at least C220, C226 and C229. In preferred embodiments, the monomeric protein comprises substitutions at all three of C220, C226 and C229 (numbering according to the EU system).

The Fc region of the fusion protein may therefore comprise one or more amino acid substitutions compared to naturally occurring Fc sequences, which prevent or reduce the ability of the Fc region to homodimerise. Preferably, the amino acid substitutions comprise one or more substitutions of the cysteine residues so as to prevent the formation of disulphide bonds between Fc molecules. The cysteine residues of the Fc region may be substituted to any other amino acid residue, optionally to glycine, serine, alanine, lysine and glutamic acid, preferably glycine or serine.

The cysteine residues for substitution are preferably one or more of the cysteine residues located in the region of the Fc region which corresponds to the hinge region of an immunoglobulin. Examples of the IgG1 hinge regions, and variations thereof including cysteine to serine substitutions are provided herein in Table 3. The hinge region of an immunoglobulin (eg of IgG1) comprises three cysteine residues (which are number C220, C226 and C229 according to EU numbering. Accordingly, in any embodiment, at least one, at least two, or all three of the cysteine residues in the immunoglobulin hinge region are substituted. Preferably, at least two or all three of the cysteine residues are substituted. More preferably, all cysteine residues in the Fc region, such as the hinge region, are substituted. In particularly preferred embodiments, at least one of C226 and C229 are substituted, preferably wherein both C226 and C229 are substituted.

Accordingly, in preferred embodiments, the fusion protein comprises a hinge region for linking the a dysfunctional P2X₇ receptor epitope moiety and Fc region of an antibody, wherein the hinge region comprises an amino acid sequence that corresponds to any of the sequences set forth in SEQ ID NOs: 76 to 113, or 136 to 137 or 141 or 142.

In further embodiments, the fusion protein region may comprise an Fc region corresponding to an Fc “hole” or “knob” for use in a “knob-in-hole” heterodimer. The use of such Fc sequences is known in the art and provides for an asymmetric heterodimeric molecule comprising a fusion protein with a single copy of the epitope moiety as described herein and Fc region, bound to a further Fc region that does not comprise the epitope moiety.

The skilled person will be familiar with technology and Fc sequences for enabling the formation of so-called monomeric fusion proteins, including although not limited to the use of the “knobs-into-holes” IgG1 format (Ridgway et al., (1996), Protein Eng, 9: 617-621). Such approaches in the context of the present invention, enable expression and purification of a heterodimeric fusion protein with only one copy of the peptide epitope (eg an epitope moiety derived from the E200 epitope as herein described), per molecule. Examples of the “knob-into-hole” Fc pairing is provided herein in SEQ ID NOs: 157 and 159 (knob and hole, respectively), 158 and 159, respectively, 160 and 162 (hole and knob, respectively) and 161 and 162, respectively. Accordingly, in any embodiment, the present invention provides a fusion protein comprising the amino acid sequence of any of SEQ ID NOs: 2 to 69 and 122, linked to an Fc region as defined in SEQ ID NO: 160 or 162, wherein the fusion protein is capable of forming a heterodimer with an Fc region that does not comprise an E200 peptide moiety.

As such, a fusion protein of the invention is preferably one that is capable of forming a heterodimeric molecule that comprises a single E200-containing amino acid sequence. (In other words, the Fc portion of the fusion protein may form a heterodimer with an Fc region of an antibody that does not comprise an E200 peptide fused thereto).

In further embodiments, the Fc region may comprise one or more substitutions for ablating or reducing effector function, such as to reduce binding and activation via the FcR as further described below.

The term “Fc region” herein is used to define a C-terminal region of an immunoglobulin heavy chain that contains at least a portion of the constant region. In other words, the Fc region contains two heavy chain fragments comprising the C_{H}2 and C_{H}3 domains of an antibody. In the context of the present invention, the Fc region comprises two heavy chain fragments, preferably the CH2 and CH3 domains of said heavy chain. The two heavy chain fragments are held together by two or more disulfide bonds and by hydrophobic interactions of the C_{H}3 domains. The heavy chain constant domains that correspond to the different classes of immunoglobulins are called α, δ, ε, γ, and μ, respectively.

In some aspects, the fusion protein does not exhibit any effector function or any detectable effector function. “Effector functions” or “effector activities” refer to those biological activities attributable to the Fc region of an antibody, which vary with the antibody isotype. Examples of antibody effector functions include: C1q binding and complement dependent cytotoxicity (CDC); Fc receptor binding; antibody dependent cell-mediated cytotoxicity (ADCC); phagocytosis; down regulation of cell surface receptors (e.g. B cell receptor); and B cell activation. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991).

Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96© non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006); WO 2013/120929 A1).

In preferred embodiments, the Fc fusion proteins of the invention comprise Fc regions with reduced effector function. Fc regions with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581). For example, an antibody variant may comprise an Fc region with one or more amino acid substitutions which diminish FcγR binding, e.g., substitutions at positions 234 and 235 of the Fc region (EU numbering of residues). For example, the substitutions are L234A and L235A (LALA) (See, e.g., WO 2012/130831). Further, alterations may be made in the Fc region that result in altered (i.e., diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000) (eg G236R).

Further examples of modified Fc regions include those comprising the “LALALS” (amino acid substitutions L234A/L235A/M428L/N434S as described in Zalevsky et al., (2010) Nat. Biotechnol. 28: 157-159); the LALAPG (L234A/L235A/P329G amino acid substitutions as described in Gunn et al., (2021, Immunity 54: 815).

In any embodiment, the Fc region of the Fc fusion proteins of the invention may comprise at least the “LALA” mutations (L234A and L235A) for reducing binding to FcR. The fusion protein may in addition or alternatively comprise the mutation G346R for abrogating recruitment of complement C1q.

Other Fc modifications for use in the present invention include variants that reduce or ablate binding to FcγRs and/or complement proteins, thereby reducing or ablating Fc-mediated effector functions such as ADCC, ADCP, and CDC. Such variants are also referred to herein as “knockout variants” or “KO variants”. Variants that reduce binding to FcγRs and complement are useful for reducing unwanted interactions mediated by the Fc region. Preferred knockout variants are described in US 2008-0242845 A1, published on Oct. 2, 2008, entitled “Fc Variants with Optimized Properties, expressly incorporated by reference herein. Preferred modifications include but are not limited substitutions, insertions, and deletions at positions 234, 235, 236, 237, 267, 269, 325, and 328, wherein numbering is according to the EU index. Preferred substitutions include but are not limited to 234G, 235E, 235G, 236R, 237K, 267R, 269R, 325L, and 328R, wherein numbering is according to the EU index. A preferred variant comprises 236R/328R. Variants may be used in the context of any IgG isotype or IgG isotype Fc region, including but not limited to human IgG1, IgG2, IgG3, and/or IgG4. Preferred IgG Fc regions for reducing FcγR and complement binding and reducing Fc-mediated effector functions are IgG2 and IgG4 Fc regions. Hybrid isotypes may also be useful, for example hybrid IgG1/IgG2 isotypes as described in U.S. Ser. No. 11/256,060. Other modifications for reducing FcγR and complement interactions include but are not limited to substitutions 297A, 297D, 234A, 235A, 237A, 318A, 228P, 236E, AG236, 265G, 268Q, 297Q, 309L, 330S, 331S, 327Q, 220S, 226S, 229S, 238S, 233P, 234A, and 234V, as well as removal of the glycosylation at position 297 by mutational or enzymatic means or by production in organisms such as bacteria that do not glycosylate proteins. These and other modifications are reviewed in Strohl, 2009, Current Opinion in Biotechnology 20:685-691, incorporated by reference in its entirety.

In some aspects, the Fc region of the fusion protein includes mutations to the complement (C1q) and/or to Fc gamma receptor (FcγR) binding sites. In some aspects, such mutations can render the fusion protein incapable of antibody directed cytotoxicity (ADCC) and complement directed cytotoxicity (CDC).

The Fc region as used in the context of the present invention preferably does not trigger cytotoxicity such as antibody-dependent cellular cytotoxicity (ADCC) or complement dependent cytotoxicity (CDC).

In some aspect, the Fc region may comprise one or more substitutions for reducing affinity for the FcRn, and thereby reduce serum or circulating half-life of the fusion protein. Substitutions for reducing affinity to FcRn are known in the art and are described for example in Ward et al., (2015), Mol. Immunol., 67: 131-141 and Grevys et al., (2015), 194: 5497-5508. Examples of substitutions include substitutions at one or more of Ile253, His310 and His435, such as 1253A and H310A and H435A.

The term “Fc region” also includes native sequence Fc regions and variant Fc regions. The Fc region may include the carboxyl-terminus of the heavy chain. Antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991. Amino acid sequence variants of the Fc region of an antibody may be contemplated. Amino acid sequence variants of an Fc region of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the Fc region of the antibody. Any combination of deletion, insertion, and substitution can be made to arrive at the final construct, provided that the final construct possesses the desired characteristics, e.g., inducing or supporting an anti-inflammatory response.

The Fc region of the antibody may be an Fc region of any of the classes of antibody, such as IgA, IgD, IgE, IgG, and IgM. The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. Accordingly, as used in the context of the present invention, the antibody may be an Fc region of an IgG. For example, the Fc region of the antibody may be an Fc region of an IgG1, an IgG2, an IgG2b, an IgG3 or an IgG4. In some aspects, the fusion protein of the present invention comprises an IgG of an Fc region of an antibody. In the context of the present invention, the Fc region of the antibody is an Fc region of an IgG, preferably IgG1.

### Linker Region Between the Dysfunctional P2X₇ Receptor Epitope and Fc Region

The dysfunctional P2X₇ receptor epitope and Fc region of an antibody (or serum albumin, transferrin, a carboxy-terminal peptide of chorionic gonadotropin (CG) p chain, a non-exact repeat peptide sequence, a polypeptide sequence composed of proline-alanine-serine polymer, an elastin-like peptide (ELP) repeat sequence), a homopolymer of glycine residues or a gelatin-like protein) may be joined directly or via a linker sequence. The linker sequence may be a spacer sequence as herein defined or as exemplified in Table 1 or 3. Alternatively, the linker sequence may be any amino acid based linker sequence commonly in use in the field.

A linker is usually a peptide having a length of up to 20 amino acids although may be up to 50 amino acids in length. The term “linked to” or “fused to” refers to a covalent bond, e.g., a peptide bond, formed between two moieties. Accordingly, in the context of the present invention the linker may have a length of 1, 2, 3, 4, 5, 6, 7, 8, 9, 10, 11, 12, 13, 14, 15, 16, 17, 18, 19, 20, 21 or 22 or more amino acids. For example, the herein provided fusion protein may comprise a linker between the epitope of a dysfunctional P2X₇ receptor and the Fc region of the antibody, such as between the N-terminus of the Fc regions and the C-terminus of dysfunctional P2X₇ receptor epitope. As another example, the herein provided fusion protein may comprise a linker between the dysfunctional P2X₇ receptor epitope and the Fc region of the antibody, such as between the C-terminus of the Fc regions and the N-terminus of the dysfunctional P2X₇ receptor epitope moiety. Particularly, the dysfunctional P2X₇ receptor epitope moiety may be fused via a linker at the C-terminus to the N-terminus of the Fc region. Such linkers have the advantage that they can make it more likely that the different polypeptides of the fusion protein fold independently and behave as expected. Thus, in the context of the present invention the dysfunctional P2X₇ receptor epitope moiety and the Fc region of an antibody may be comprised in a single-chain multi-functional polypeptide.

In some aspects, the fusion protein of the present invention or polypeptide for use according to the invention, includes a peptide linker. In some aspects, the peptide linker links a dysfunctional P2X₇ receptor epitope moiety with an Fc region of an antibody. In some aspects, the peptide linker can include the amino acid sequence Gly-Gly-Ser (GGS), Gly-Gly-Gly-Ser (GGGS) or Gly-Gly-Gly-Gly-Ser (GGGGS). In some aspects, the peptide linker can include the amino acid sequence GGGGS (a linker of 6 amino acids in length) or even longer. The linker may a series of repeating glycine and serine residues (GS) of different lengths, i.e., (GS)n where n is any number from 1 to 15 or more. For example, the linker may be (GS)3 (i.e., GSGSGS) or longer (GS)11 or longer. It will be appreciated that n can be any number including 1, 2, 3, 4, 5, 6, 7, 8, 9, 10, 11 or more. Fusion proteins having linkers of such length are included within the scope of the present invention. Preferably n is no more than 3 (ie such that when n equals 3 the linker is GSGSGS).

In further embodiments, the linker may comprise inclusion of an amino acid that provides rigidity, such as lysine. For example, in certain embodiments, the linker region may also comprise the sequence GSGK.

The peptide linker may consist of a series of repeats of Thr-Pro (TP) comprising one or more additional amino acids N and C terminal to the repeat sequence. For example, the linker may comprise or consist of the sequence GTPTPTPTPTGEF (also known as the TP5 linker). In further aspects, the linker may be a short and/or alpha-helical rigid linker (e.g. A(EAAAK)3A, PAPAP or a dipeptide such as LE or CC).

In further embodiments, as an alternative or in addition to a glycine-serine-based linker region as described above, the fusion protein may comprise a dysfunctional P2X₇ receptor epitope moiety, linked to an Fc region of an antibody, via a hinge region. The linking between the dysfunctional P2X₇ receptor epitope moiety and the Fc region may comprise a combination of hinge region and linker regions.

Examples of suitable hinge regions include hinge regions derived from immunoglobulins. The hinge region may be derived from an IgG1, IgG2, IgG3 or IgG4, and may comprise one or more amino acid substitutions, (for example to prevent or reduce the likelihood of disulphide bridge formation). Alternative hinge sequences may be derived from alternative immunoglobulin domains, CD8A, CD8B, CD4 or CD28, TRAC, TRBC, TRGC, TRDC.

Further linker sequences may also include any sequence of any length of CL/CH1 domain but not all residues of CL/CH1 domain; for example the first 5-12 amino acid residues of the CL/CH1 domains. Linkers can be derived from immunoglobulin heavy chains of any isotype, including for example Cγ1, Cγ2, Cγ3, Cγ4, Cα1, Cα2, Cδ, Cε, and Cμ. Linkers can be derived from immunoglobulin light chain, for example Cκ or Cλ. Linker sequences may also be derived from other proteins such as Ig-like proteins (e.g. TCR, FcR, KIR), hinge region-derived sequences, and other natural sequences from other proteins.

Table 3 below provides non-limiting examples of suitable hinge regions for use in joining the dysfunctional P2X₇ receptor epitope moiety and Fc regions, in the molecules of the invention.

It will be appreciated that the dysfunctional P2X₇ receptor epitope moiety may be joined to the Fc regions (or other protein sequence as herein defined) by more than one linker and/or more than one hinge region. For example, the fusion protein may comprise (N to C terminus), the dysfunctional P2X₇ receptor epitope moiety, conjugated directly to the Fc region. Alternatively, the fusion protein may comprise the dysfunctional P2X₇ receptor epitope moiety, followed by a linker region, then the Fc region. Further still, the fusion protein may comprise the dysfunctional P2X₇ receptor epitope moiety, followed by a linker region, then a hinge region, and then the Fc region. In a further embodiment still, the fusion protein may comprise the dysfunctional P2X₇ receptor epitope moiety, followed by a linker region, then a hinge region, a further linker region, then the Fc region. Of course, the skilled person will appreciate that the alternative configuration is possible (ie wherein the dysfunctional P2X₇ receptor epitope moiety is joined to the C terminus of the Fc region, via one or more linker and/or hinge regions.

TABLE 3
further exemplary linker/hinge region sequences

| Amino acid sequence | SEQ ID NO: | Description |
|---|---|---|
| EPKSSDKTHTSPPSP | 76 | Sequences derived from IgG1 (ie |
| EPKSSDKTHTSPP | 77 | modified IgG1 hinge regions) |
| EPKSSDKTHTSPPP | 78 | |
| EPKSSDKTHT | 79 | |
| DKTHTSPPSP | 80 | |
| DKTHTSPP | 81 | |
| EPKSDKTHTPPP | 82 | |
| EPKSDKTHTSPPSP | 83 | |
| EPKSGDKTHTGPPGP | 84 | |
| EPKSGDKTHTGPP | 85 | |
| EPKSGDKTHTGPPP | 86 | |
| EPKSGDKTHT | 87 | |
| DKTHTGPPGP | 88 | |
| DKTHTGPP | 89 | |
| EPKSDKTHTGPPGP | 90 | |
| EPKSGDKTHTSPPSP | 91 | |
| EPKSGDKTHTGPPSP | 92 | |
| EPKSSDKTHTGPPGP | 93 | |
| EPKSSDKTHTGPPSP | 94 | |
| EPKSSDKTHTGPP | 95 | |
| EPKSGDKTHTSPP | 96 | |
| EPKSTDKTHTTPPTP | 97 | |
| EPKSTDKTHTTPP | 98 | |
| EPKSTDKTHTTPPP | 99 | |
| EPKSTDKTHT | 100 | |
| DKTHTTPPTP | 101 | |
| DKTHTTPP | 102 | |
| EPKSDKTHTTPPTP | 103 | |
| EPKSSDKTHTTPPTP | 104 | |
| EPKSSDKTHTSPPTP | 105 | |
| EPKSADKTHTLPPMP | 106 | |
| EPKSVDKTHTLPPIP | 107 | |
| EPKSLDKTHTAPPAP | 108 | |
| EPKSVDKTHTAPP | 109 | |
| EPKSIDKTHTLPP | 110 | |
| DKTHTAPPLP | 111 | |
| DKTHTVPPLP | 112 | |
| EPKSSDKTHTSP | 113 | |
| EPKSCDKTHTCPPCPAP | 114 | Wild type IgG1 |
| ERKCCVECPPCP | 115 | Mutated IgG2 |
| ERKXXVEXPPXP | 116 | Mutated IgG2 |
| ERKXXVEXPP | 117 | Mutated IgG2 |
| VEXPPXP | 118 | Mutated IgG2 |
| ELKTPLGDTTHTCPRCP | 119 | Wild-type IgG3 |
| ELKTPLGDTTHTXPRXP | 120 | Mutated IgG3 |
| ELKTPLGDTTHTXPR | 121 | Mutated IgG3 |
| ELKTPLGDTTHT | 123 | Mutated IgG3 |
| EPKSCDTPPPCPRCP | 124 | Wild-type IgG3 |
| EPKSXDTPPPXPRXP | 125 | Mutated IgG3 |
| EPKSXDTPPPXPR | 126 | Mutated IgG3 |
| EPKSXDTPPP | 127 | Mutated IgG3 |
| DTPPPXPRXP | 128 | Mutated IgG3 |
| ESKYGPPCPSCP | 129 | Wild-type IgG4 |
| EXKYGPPCPXCP | 130 | Mutated IgG4 |
| EXKYGPPCP | 131 | Mutated IgG4 |
| KYGPPCPXCP | 132 | Mutated IgG4 |
| EPKSCDKTHTCP | 133 | Wild type IgG1 (v2) |
| EPKSCDTPPPCP | 134 | Wild type IgG3 (v2) |
| ESKYGPPSPSSP | 135 | Mutated igG4 |
| ERKSSVESPPSP | 136 | Mutated igG2 |
| EPKSSDTPPPSP | 137 | Mutated igG3 |

In certain embodiments, the dysfunctional P2X₇ receptor epitope moiety is directly fused to the Fc region of an antibody (or serum albumin, transferrin, a carboxy-terminal peptide of chorionic gonadotropin (CG) β chain, a non-exact repeat peptide sequence, a polypeptide sequence composed of proline-alanine-serine polymer, an elastin-like peptide (ELP) repeat sequence), a homopolymer of glycine residues or a gelatin-like protein), such that there is no linker between the two regions of the fusion protein or polypeptide.

In certain embodiments, the linker joining the dysfunctional P2X₇ receptor epitope moiety and the Fc region of an antibody (or serum albumin, transferrin, a carboxy-terminal peptide of chorionic gonadotropin (CG) p chain, a non-exact repeat peptide sequence, a polypeptide sequence composed of proline-alanine-serine polymer, an elastin-like peptide (ELP) repeat sequence), a homopolymer of glycine residues or a gelatin-like protein), is a cleavable linker.

Cleavable linkers are well known in the art, and include for example, the sequence defined in SEQ ID NO: 144 and which defines a cleavage site for Human Rhinovirus 3C protease. Proteases for sue in cleaving such cleavage sites are also readily available from commercial providers (eg: Pierce HRV 3C Protease.

Other known cleavable linkers and other linker which may be used in accordance with the present invention are disclosed in Chen et al., (2013) Adv. Drug. Deliv. Rev. 65: 1357-1369; the contents of which are incorporated herein by reference.

### Receptors and Immune Cells Expressing Same

The present invention finds application in methods for enriching for subpopulations of immune cells that express receptors comprising antigen binding domains for binding to tumour associated and tumour specific antigens, such dysfunctional P2X₇ receptor. The receptor is preferably a chimeric antigen receptor (CAR) or variant thereof. The receptor may also be a modified TCR.

The antigen-recognition domain of the receptor preferably recognises a target antigen expressed on a cancer cell. It will be appreciated that any number of different immune cells expressing different antigen-recognition domains for binding different target antigens may be utilised in accordance with the present invention, although it will be necessary to utilise a molecule comprising an epitope that competes for binding to the cellular immunotherapeutic.

For example, the immune cell may comprise a receptor with an antigen-recognition domain for binding to any one of: CD33 (Siglec-3), CD123 (IL3RA), CD135 (FLT-3), CD44 (HCAM), CD44V6, CD47, CD184 (CXCR4), CLEC12A (CLL1), FRp, MICA/B, CD305 (LAIR-1), CD366 (TIM-3), CD96 (TACTILE), CD133, CD56, CD29 (ITGB1), CD44 (HCAM), CD47 (IAP), CD66 (CEA), CD112 (Nectin2), CD117 (c-Kit), CD146 (MCAM), CD155 (PVR), CD171 (LI CAM), CD221 (IGF1), CD227 (MUC1), CD243 (MRD1), CD246 (ALK), CD271 (LNGFR), CD19, CD20, GD2, and especially EGFR, mesothelin, GPC3, MUC1, HER2, GD2, CEA, EpCAM, LeY, CD276 and PCSA.

In certain embodiments, the immune cell expresses a CAR (or variant thereof) for binding dysfunctional P2X₇ receptor. The extracellular part of the CAR or variant thereof may comprise an nfP2X₇ binding domain that recognises the E200 (or E300 or E200-300 composite) epitope as disclosed herein.

In general, a CAR, variant thereof, or TCR, may comprise an extracellular domain (extracellular part) comprising the antigen binding domain, a transmembrane domain and an intracellular signaling domain. The extracellular domain may be linked to the transmembrane domain by a linker. The extracellular domain may also comprise a signal peptide. Preferably, the extracellular part of the CAR, variant thereof, or TCR comprises an nfP2X₇ binding domain that recognises the E200 (or E300 or E200-300 composite) epitope as disclosed herein.

Typically, the antigen-recognition domain of the CAR or TCR includes a binding polypeptide that includes amino acid sequence homology to one or more complementarity determining regions (CDRs) of an antibody that binds to a dysfunctional P2X₇ receptor. In any embodiment, the binding polypeptide includes amino acid sequence homology to the CDR1, 2 and 3 domains of the V_{H} and/or V_{L} chain of an antibody that binds to a dysfunctional P2X₇ receptor. As will be appreciated, the CAR will preferably be able to recognise the same dysfunctional P2X₇ receptor epitope moiety that is present on the fusion proteins of the invention.

Although it will be appreciated that the methods of the invention find application for enriching cells expressing any CAR for binding to dysfunctional P2X₇ receptor, in preferred embodiments, the binding polypeptide of the CAR comprises the amino acid sequence of the CDRs of the V_{H} and/or V_{L} chain of an antibody described in PCT/AU2002/000061 or PCT/AU2002/001204 (or in any one of the corresponding U.S. Pat. Nos. 7,326,415, 7,888,473, 7,531,171, 8,080,635, 8,399,617, 8,709,425, 9,663,584, or U.S. Pat. No. 10,450,380), PCT/AU2007/001540 (or in corresponding U.S. Pat. No. 8,067,550), PCT/AU2007/001541 (or in corresponding US publication US 2010-0036101), PCT/AU2008/001364 (or in any one of the corresponding U.S. Pat. Nos. 8,440,186, 9,181,320, 9,944,701 or U.S. Pat. No. 10,597,451), PCT/AU2008/001365 (or in any one of the corresponding U.S. Pat. No. 8,293,491 or U.S. Pat. No. 8,658,385), PCT/AU2009/000869 (or in any one of the corresponding U.S. Pat. No. 8,597,643, 9,328,155 or 10,238,716), PCT/AU2010/001070 (or in any one of the corresponding publications WO/2011/020155, U.S. Pat. No. 9,127,059, 9,688,771, or 10,053,508), and PCT/AU2010/001741 (or in any one of the corresponding publications WO 2011/075789 or U.S. Pat. No. 8,835,609) the entire contents of which are hereby incorporated by reference. Preferably the antibody comprises the CDR amino acid sequences of PEP2-2-1 described in PCT/AU2010/001070 (or in any one of the corresponding U.S. Pat. Nos. 9,127,059, 9,688,771, or U.S. Pat. No. 10,053,508) or BPM09 described in PCT/AU2007/001541 (or in corresponding US publication US 2010-0036101) and produced by the hybridoma AB253 deposited with the European Collection of Cultures (ECACC) under Accession no. 06080101.

In further embodiments, the binding polypeptide of the CAR comprises the amino acid sequence of the V_{H} and/or V_{L} chains of an antibody described in PCT/AU2002/000061 or PCT/AU2002/001204 (or in any one of the corresponding U.S. Pat. No. 7,326,415, 7,888,473, 7,531,171, 8,080,635, 8,399,617, 8,709,425, 9,663,584, or 10,450,380), PCT/AU2007/001540 (or in corresponding U.S. Pat. No. 8,067,550), PCT/AU2007/001541 (or in corresponding US publication US 2010-0036101), PCT/AU2008/001364 (or in any one of the corresponding U.S. Pat. No. 8,440,186, 9,181,320, 9,944,701 or 10,597,451), PCT/AU2008/001365 (or in any one of the corresponding U.S. Pat. No. 8,293,491 or 8,658,385), PCT/AU2009/000869 (or in any one of the corresponding U.S. Pat. No. 8,597,643, 9,328,155 or 10,238,716), PCT/AU2010/001070 (or in any one of the corresponding publications WO/2011/020155, U.S. Pat. No. 9,127,059, 9,688,771, or 10,053,508), and PCT/AU2010/001741 (or in any one of the corresponding publications WO 2011/075789 or U.S. Pat. No. 8,835,609) the entire contents of which are hereby incorporated by reference. Preferably the antibody comprises the CDR amino acid sequences of PEP2-2-1 described in PCT/AU2010/001070 (or in any one of the corresponding U.S. Pat. No. 9,127,059, 9,688,771, or 10,053,508) or BPM09 described in PCT/AU2007/001541 (or in corresponding US publication US 2010-0036101) and produced by the hybridoma AB253 deposited with the European Collection of Cultures (ECACC) under Accession no. 06080101.

In further embodiments still, the binding polypeptide of the CAR comprises the amino acid sequence of an antibody or fragment thereof described in PCT/AU2002/000061 or PCT/AU2002/001204 (or in any one of the corresponding U.S. Pat. No. 7,326,415, 7,888,473, 7,531,171, 8,080,635, 8,399,617, 8,709,425, 9,663,584, or 10,450,380), PCT/AU2007/001540 (or in corresponding U.S. Pat. No. 8,067,550), PCT/AU2007/001541 (or in corresponding US publication US 2010-0036101), PCT/AU2008/001364 (or in any one of the corresponding U.S. Pat. No. 8,440,186, 9,181,320, 9,944,701 or . 10,597,451), PCT/AU2008/001365 (or in any one of the corresponding U.S. Pat. No. 8,293,491 or 8,658,385), PCT/AU2009/000869 (or in any one of the corresponding U.S. Pat. No. 8,597,643, 9,328,155 or 10,238,716), PCT/AU2010/001070 (or in any one of the corresponding publications WO/2011/020155, U.S. Pat. No. 9,127,059, 9,688,771, or 10,053,508), and PCT/AU2010/001741 (or in any one of the corresponding publications WO 2011/075789 or U.S. Pat. No. 8,835,609) the entire contents of which are hereby incorporated by reference. Preferably the antibody comprises the CDR amino acid sequences of PEP2-2-1 described in PCT/AU2010/001070 (or in any one of the corresponding U.S. Pat. No. 9,127,059, 9,688,771, or 10,053,508) or BPM09 described in PCT/AU2007/001541 (or in corresponding US publication US 2010-0036101) and produced by the hybridoma AB253 deposited with the European Collection of Cultures (ECACC) under Accession no. 06080101.

The CAR typically also comprises a signal peptide. A “signal peptide” refers to a peptide sequence that directs the transport and localisation of the protein within a cell, e.g. to a certain cell organelle (such as the endoplasmic reticulum) and/or the cell surface.

Generally, an “antigen binding domain” (or antigen recognition domain) refers to the region of the CAR that specifically binds to an antigen (and thereby is able to target a cell containing the antigen). A CAR may comprise one or more antigen binding domains. Generally, the targeting regions on the CAR are extracellular. The antigen binding domain may comprise an antibody or an antibody binding fragment thereof. The antigen binding domain may comprise, for example, full length heavy chain, Fab fragments, single chain Fv (scFv) fragments, divalent single chain antibodies or diabodies. Any molecule that binds specifically to a given antigen such as affibodies or ligand binding domains from naturally occurring receptors may be used as an antigen binding domain. Often the antigen binding domain is a scFv. Normally, in an scFv the variable regions of an immunoglobulin heavy chain and light chain are fused by a flexible linker to form a scFv. Such a linker may be for example the “(G₄/S₁)₃-linker” and variations thereof but the skilled person will appreciate that various linker sequences and formats may be used.

CARs may also comprise a “hinge” region (sometimes called a spacer region or linker region) joining the antigen binding domain to the transmembrane domain. This is typically a hydrophilic region that is between the antigen binding domain and the transmembrane domain. A CAR may comprise an extracellular hinge domain but it is also possible to leave out such a hinge. The hinge region may include for example, Fc fragments of antibodies or fragments thereof, hinge regions of antibodies or fragments thereof, CH2 or CH3 regions of antibodies, accessory proteins, artificial hinge sequences or combinations thereof. One example of a hinge region is the CD8alpha hinge.

The transmembrane domain of the CAR may be derived from any desired natural or synthetic source for such a domain. When the source is natural, the domain may be derived from any membrane-bound or transmembrane protein. The transmembrane domain may be derived for example from CD8alpha or CD28. When the key signalling and antigen recognition modules (domains) are on two (or even more) polypeptides, then the CAR may have two (or more) transmembrane domains. The splitting of key signalling and antigen recognition modules enables small molecule-dependent, titratable and reversible control over CAR cell expression (Wu et al, 2015, Science 350: 293-303) due to small molecule-dependent heterodimerising domains in each polypeptide of the CAR.

The cytoplasmic domain (or the intracellular signaling domain) of the CAR is responsible for activation of at least one of the normal effector functions of the immune cell in which the CAR is expressed. “Effector function” means a specialised function of a cell, e.g. in a T cell an effector function may be cytolytic activity or helper cell activity including the secretion of cytokines. The intracellular signalling domain refers to the part of a protein that transduces the effector function signal and directs the cell expressing the CAR to perform a specialised function. The intracellular signalling domain may include any complete, mutated or truncated part of the intracellular signalling domain of a given protein sufficient to transduce a signal that initiates or blocks immune cell effector functions.

The function of the intracellular domains may be pro- or anti-inflammatory and/or immunomodulatory, or a combination of such.

Examples of intracellular signalling domains for use in the CARs include the cytoplasmic signaling sequences of the T cell receptor (TCR) and co-receptors that initiate signal transduction following antigen receptor engagement.

Primary cytoplasmic signalling sequences that act in a stimulatory manner may contain ITAMs (immunoreceptor tyrosine-based activation motifs) signalling motifs.

Examples of ITAM containing primary cytoplasmic signalling sequences often used in CARs are those derived from TCR zeta (CD3 zeta), FcR gamma, FcR beta, CD3 gamma, CD3 delta, CD3 epsilon, CD5, CD22, CD79a, CD79b and CD66d. Most prominent is the sequence derived from CD3 zeta.

The cytoplasmic domain of the CAR may be designed to comprise the CD3-zeta signaling domain by itself or combined with any other desired cytoplasmic domain(s). The cytoplasmic domain of the CAR can comprise a CD3 zeta chain portion and a co-stimulatory signalling region. The co-stimulatory signalling region refers to a part of the CAR comprising the intracellular domain of a co-stimulatory molecule. A co-stimulatory molecule is a cell surface molecule other than an antigen receptor or their ligands that is required for an efficient response of lymphocytes to an antigen. Examples for a co-stimulatory molecule are CD27, CD28, 4-1 BB (CD137), OX40, CD30, CD40, PD-1, ICOS, lymphocyte function-associated antigen-1 (LFA-1), CD2, CD7, LIGHT, NKG2C and B7-H3.

In some embodiments, the activation receptor (from which a portion of signalling domain is derived) is the CD3 co-receptor complex or is an Fc receptor.

In some embodiments, the co-stimulatory receptor (from which a portion of signalling domain is derived) is selected from the group consisting of CD27, CD28, CD-30, CD40, DAP10, OX40, 4-1BB (CD137) and ICOS.

In some embodiments, the co-stimulatory receptor (from which a portion of signalling domain is derived) is selected from the group consisting of CD28, OX40 or 4-1BB.

The cytoplasmic signalling sequences within the cytoplasmic signalling part of the CAR may be linked to each other with or without a linker in a random or specified order. A short oligo- or polypeptide linker, which is preferably between 2 and 10 amino acids in length, may form the linkage. A prominent linker is the glycine-serine doublet.

As an example, the cytoplasmic domain may comprise the signalling domain of CD3-zeta and the signalling domain of CD28. In another example the cytoplasmic domain may comprise the signalling domain of CD3-zeta and the signalling domain of CD27. In a further example, the cytoplasmic domain may comprise the signalling domain of CD3-zeta, the signalling domain of CD28, and the signalling domain of CD27.

As aforementioned, either the extracellular part or the transmembrane domain or the cytoplasmic domain of a CAR may also comprise a heterodimerising domain for the aim of splitting key signalling and antigen recognition modules of the CAR.

The CAR which binds to a fusion protein of the invention or polypeptide for use according to invention, e.g., a CAR comprising an nfP2X₇ E200 binding domain, may be designed to comprise any portion or part of the above-mentioned domains as described herein in any order and/or combination resulting in a functional CAR.

The affinity at which the dysfunctional P2X₇ receptor binding domain of the CAR binds to the nfP2X₇ recognition site E200 of fusion protein of the invention or polypeptide for use according to the invention, can vary, but generally the binding affinity may be in the range of approximately 100 μM, approximately 10 μM, approximately 1 μM, approximately 100 nM, approximately 10 nM, or approximately 1 nM, preferably at least about 10 μM or 1 μM. In preferred embodiments, the binding affinity is at least about 1 nM or at least about 10 nM.

The receptor (such as a CAR, variant thereof, or TCR, or variant thereof) is typically expressed by an immune cell.

The immune cell may be an “engineered cell”, “genetically modified cell”, or “immune effector cell” as described herein. Further, the immune cell may be an immune cell precursor that is capable of differentiating into an immune cell. A cell that is capable of differentiating into an immune cell (e.g. T cell that will express the dysfunctional P2X₇ CAR) may be a stem cell, multi-lineage progenitor cell or induced pluripotent stem.

The immune cell may be a leukocyte, a Peripheral Blood Mononuclear Cell (PBMC), a lymphocyte, a T cell (including a CD4+ T cell or a CD8+ T cell), a natural killer cell, a natural killer T cell, or a γδT cell.

In any embodiment, the immune cell may be a T cell, wherein optionally said T cell does not express TcRap, PD1, CD3 or CD96 (e.g. by way of knocking down or knocking out one of these genes on a genetic level or functional level).

In any embodiment, the immune cell optionally does not express accessory molecules that can be checkpoint, exhaustion or apoptosis-associated signalling receptors as well as ligands such as PD-1, LAG-3, TIGIT, CTLA-4, FAS-L and FAS-R, (e.g. by way of knocking out one of these genes on a genetic level or functional level).

In some embodiments, the immune cell includes two or more different receptors (e.g., two or more CARs, or variants thereof). The CARs may bind to different epitopes on the same target molecule (e.g., different epitopes on dysfunctional P2X₇ receptor). Alternatively, the CARs may bind different target molecules, such that only one of the CARs binds to dysfunctional P2X₇ receptors.

As used herein, the term “different CARs” or “different chimeric antigen receptors” refers to any two or more CARs that have either non-identical antigen-recognition and/or non-identical signalling domains. In one example, “different CARs” includes two CARs with the same antigen-recognition domains (e.g. both CARs may recognise a dysfunctional P2X₇ receptor), but have different signalling domains, such as one CAR having a signalling domain with a portion of an activation receptor and the other CAR having a signalling domain with a portion of an co-stimulatory receptor. As will be understood, at least one of the two or more CARs within this embodiment will have an antigen-recognition domain that recognises the dysfunctional P2X₇ receptor and the other CAR(s) may take any suitable form and may be directed against any suitable antigen.

### Methods for Enriching Immune Cells

It will be well within the purview of the skilled person to confirm the ability of a given fusion protein or polypeptide, as defined herein to be bound by the relevant receptor. For example in the context of nfP2X₇ receptor-binding CARs, the skilled person will be able to use routine techniques to confirm binding of the polypeptide (or series of polypeptides) by the CAR, and thereby determine the suitability of the polypeptide for use in methods of the invention.

It will be understood that the methods of the invention can be used to isolate or enrich any population of immune cells that express a receptor (including a CAR), which comprises an antigen binding domain that recognizes the epitope of the dysfunctional P2X₇ receptor contained on the fusion protein.

Moreover, upon contacting the fusion protein or polypeptide of the invention with any population of cells comprising the target cells (ie cells capable of binding to the dysfunctional P2X₇ receptor epitope on the fusion protein), the skilled person can make use of routine laboratory techniques to release the fusion protein/cell complex from the capture agent, and thereby obtain an enriched sample of cells.

The use of magnetic micro- or macro-beads for isolating cell and protein populations is well known to the persons skilled in the art. The mean diameter of the beads can range from 10 nm to 10 μm. Biocompatible magnetic particles are commercially available and consist of, for example, forms of magnetically iron oxide coated by a shell of dextran molecules or silica. The solid support may also be polymers containing magnetic materials. Commercially available forms of such beads are also readily available and known, including MicroBeads (Miltenyi Biotec) for use in conjunction with MACS® columns and Dynabeads magnetic beads (Applied Biosystems).

The use of microbeads (50 nm diameter) is advantageous over the use of macrobeads (1-5 μm diameter) since these can be injected directly into the patient requiring treatment with the immune cells. Accordingly, in certain embodiments, it is not necessary to isolate the immune cells from the polypeptide/cell complex according to the second aspect of the invention, and the complex can be directly administered. In alternative embodiments, where the micro or macro beads are linked to the fusion protein or polypeptide via a biotin moiety, it may be possible to separate the beads from the fusion protein or polypeptide through addition of excess of free biotin.

Further still, the skilled person will appreciate that more than one round of enrichment may be performed in order to increase the purity of the final cell composition.

In some embodiments, the present invention provides a method of enriching cells expressing a chimeric antigen receptor (CAR) over cells not expressing the CAR in a composition, which comprises contacting the cells with a fusion protein as described herein. In some embodiments, the cells expressing the CAR are cells transduced with a nucleic acid molecule encoding the CAR. In some embodiments, the cells expressing the CAR are clones of the cells transduced with a nucleic acid molecule encoding the CAR that express the CAR.

### Compositions and Uses Thereof

The present invention also provides a composition comprising, consisting essentially of, or consisting of one or more cells expressing one or more CARs for binding to dysfunctional P2X₇ receptor, that have been enriched according to one or more of the methods of the present invention.

As used herein, a composition “comprising” one or more cells expressing one or more CARs that have been enriched according to one or more of the methods of the present invention, may contain other compounds and cells. As used herein, a composition “consisting essentially of” one or more cells expressing one or more CARs that have been enriched according to one or more of the methods of the present invention may comprise other compounds and cells so long as they do not materially change the activity or function of the cells expressing the one or more CARs in the composition. As used herein, a composition “consisting of” one or more cells expressing one or more CARs that have been enriched according to one or more of the methods of the present invention means that the composition does not contain other functional cells in addition to the one or more cells expressing one or more CARs.

Compositions consisting of one or more cells expressing one or more CARs that have been enriched according to one or more of the methods of the present invention may comprise ingredients other than cells, e.g., compounds, proteins, pharmaceutically acceptable carriers, surfactants, preservatives, etc. In some embodiments, compositions consisting of one or more cells expressing one or more CARs that have been enriched according to one or more of the methods of the present invention may contain insignificant amounts of contaminants.

In some embodiments, the amount of the one or more cells expressing one or more CARs for binding to dysfunctional P2X₇ receptor is at least about 50% of the total cells in the composition. In some embodiments, the amount of the one or more cells expressing one or more CARs is at least about 60% of the total cells in the composition. In some embodiments, the amount of the one or more cells expressing one or more CARs is at least about 70% of the total cells in the composition. In some embodiments, the amount of the one or more cells expressing one or more CARs is at least about 80% of the total cells in the composition. In some embodiments, the amount of the one or more cells expressing one or more CARs is at least about 90% of the total cells in the composition. In some embodiments, the amount of the one or more cells expressing one or more CARs is at least about 95% of the total cells in the composition. In some embodiments, the compositions according to the present invention comprise a therapeutically effective amount of the one or more cells expressing one or more CARs.

The compositions or cell populations obtained by the methods of the present invention can be used in the treatment of a disease or condition characterized by the expression of dysfunctional P2X₇ receptor.

In one embodiment, subjects requiring treatment include those having a benign, pre-cancerous, non-metastatic tumour. In one embodiment, the cancer is pre-cancerous or pre-neoplastic.

In one embodiment, the cancer is a secondary cancer or metastasis. The secondary cancer may be located in any organ or tissue, and particularly those organs or tissues having relatively higher haemodynamic pressures, such as lung, liver, kidney, pancreas, bowel and brain. The secondary cancer may be detected in the ascites fluid and/or lymph nodes.

In one embodiment, the cancer may be substantially undetectable.

“Pre-cancerous” or “preneoplasia” generally refers to a condition or a growth that typically precedes or develops into a cancer. A “pre-cancerous” growth may have cells that are characterised by abnormal cell cycle regulation, proliferation, or differentiation, which can be determined by markers of cell cycle.

The cancer may be a solid or a “liquid” tumour. In other words, the cancer may be growth in a tissue (carcinoma, sarcoma, adenomas etc) or it may be a cancer present in bodily fluid such as in blood or bone marrow (e.g., lymphomas and leukaemias).

In certain preferred embodiments, the cancer requiring treatment may be a cancer characterised by low levels of expression of dysfunctional P2X₇ receptor. Examples of such cancers include Burkitt's lymphoma. However, immunohistochemical analyses of surface expression of the dysfunctional P2X₇ (nfP2X₇) receptor on patient tumour biopsies reveals a range from 1+ to 3+ in IHC score. Samples with low expression may therefore be found in a wide range of tumour types. Examples are found in solid tumours of various types, including but not limited to neuroblastoma, colorectal cancers, lung cancers, kidney cancers, skin cancers, breast cancers, brain cancers and prostate cancer. Such differences in expression level in different tissues may be due to the formation of tumours from cells that are at an earlier state of transformation (the tissues with the highest receptor expression may be those undergoing the highest rate of proliferation).

Other examples of cancers that can be treated in accordance with the methods of the present invention include blastoma (including medulloblastoma and retinoblastoma), sarcoma (including liposarcoma and synovial cell sarcoma), neuroendocrine tumours (including carcinoid tumours, gastrinoma, and islet cell cancer), mesothelioma, schwannoma (including acoustic neuroma), meningioma, adenocarcinoma, melanoma, leukaemia or lymphoid malignancies, lung cancer including small-cell lung cancer (SCKC), non-small cell lung cancer (NSCLC), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer (including metastatic breast cancer), colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, oesophageal cancer, tumours of the biliary tract, as well as head and neck cancer.

### EXAMPLES

### Example 1: Enrichment of nfP2X₇ Receptor-Binding CAR T Cells Using Biotin-Labelled Fusion Protein

Jurkat cells and/or primary CD4+ T cells and CD8+ T cells (mixed at 1:1 ratio after enrichment) from healthy volunteer donors (Donor 12 and 57) were stably transduced by lentivirus (3rd gen LV system) to express an anti-nfP2X₇ chimeric antigen receptor (CAR), wherein the CAR comprised an antigen binding domain for binding to the E200 epitope of the P2X₇ receptor.

The Jurkat nfP2X₇ reporter cell line was grown as a single cell clone expressing the nfP2X₇-CAR by FACS sorting. The CAR constructs also contained a truncated EGFR (tEGFR) downstream a ribosomal skip site (T2A) as a marker gene, for use in enabling detection of successfully transduced cells after enrichment while the CAR receptor was still occupied by the enrichment reagents (DetR1 or DetR2 as exemplary dimeric or monomeric Fc attenuated fusion proteins and as set forth in Table 1).

To mimic the enrichment process Jurkat wildtype cells (CAR negative cells, tEGFR negative cells) were mixed with Jurkat nfP2X₇-CAR-tEGFR expressing cells at a 1:1 ratio or primary generated nfP2X₇ CAR T cell products were used. One round of positive selection was performed as outlined below.

A fusion protein comprising the sequence of SEQ ID NO: 149 was labelled with a biotin label. The Fc fusion protein was conjugated to biotin by EZ-Link™ NHS-LC-LC-Biotin (ThermoFisher, catalog number 21343) according to manufacturer instructions. The labelled Fc fusion protein was added to the mixture of untransduced and transduced cells at indicated concentrations of 1 or 2 μg/ml, and the cell/fusion protein mixture was incubated for 10 minutes at room temperature and washed in Magnetic Activated Cell Sorting (MACS) cell separation buffer (Miltenyi Biotec) according to manufacturer instructions.

Method 1: Either anti-biotin microbeads (Miltenyi Biotec, 130-090-485) were then added to this mixture, which was incubated for a further 15 minutes at 4° C. The anti-biotin microbeads are magnetic particles coated with anti-biotin antibody to enable separation of cells bound by the anti-biotin antibody coated microbeads. Separation was then performed according to manufacturer instructions.

The cell suspension was loaded onto a MS MACS® Column, (Miltenyi Biotec, 130-042-201, which is placed in the magnetic field of a MACS Separator. The magnetically labelled material (ie comprising anti-biotin bound to fusion protein and CAR T cells) is retained within the column. The flow-through (comprising cells which do not bind to the fusion protein) is discarded.

Method 2: Enrichment of cells was performed using the EasySep technology from STEMCELL Technologies according to manufacturer instructions as indicated after the primary incubation with the indicated Fc fusion protein as a comparison to the MACS technology from Miltenyi Biotec.

In both technologies MACS and EasySep the probe in the final step is removed from the magnetic field and the magnetically retained material is then eluted. The eluted material comprises those Jurkat cells or primary T cells which were successfully transduced with the lentiviral vector and express the nfP2X₇-CAR and are therefore capable of being bound by the biotin-labelled fusion protein comprising the E200 epitope of the P2X₇ receptor.

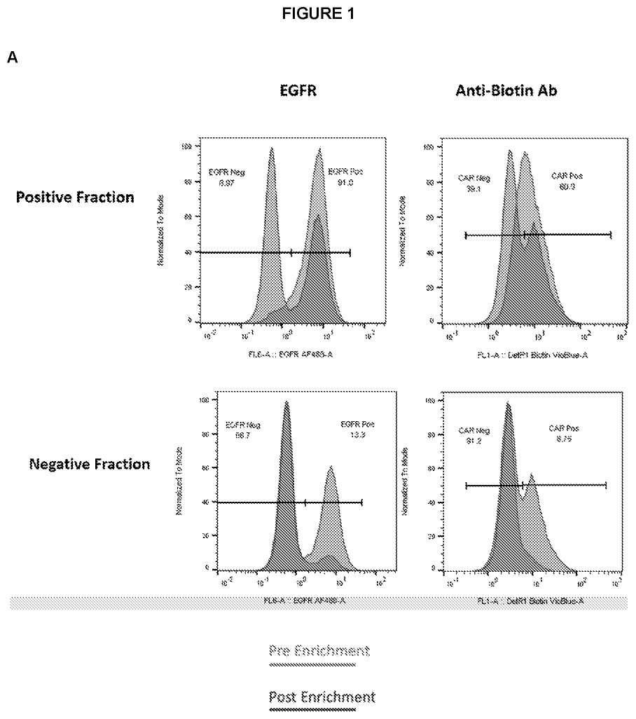

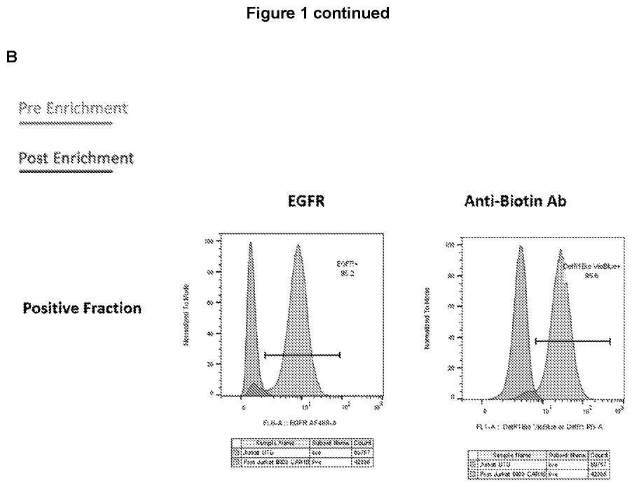

FIG. 1 shows the results of the enrichment protocol, pre- and post-enrichment, after 0 hr. and 48 hr post-enrichment of Jurkat nfP2X₇ CAR T cells, whereby tEGFR was used as the marker gene to identify the CAR expressing cells. Direct detection of nfP2X₇ CAR positive cells was done by indirect staining using the same LC-LC-biotinylated Fc attenuated fusion protein DetR1 or 2 (as defined in Table 1) and a secondary anti-biotin antibody. After a single round of positive selection, the purity of CAR expressing cells was >90%. 48 hours post enrichment (FIG. 1B) demonstrates the survival and purity was still >90%.

Similar experiments were performed using primary cells obtained from two separate donors (“donor 12” and “donor 57”). Briefly, buffy coats (PBMCs) were obtained from the donors, using standard techniques. the cells were enriched for CD4+ and CD8+ T cells using CD4+/CD8+ microbeads (MACS). After enrichment, cells were mixed at a 1:1 ratio, and then transduced with lentivirus expressing anti-nfP2X₇ CAR constructs.

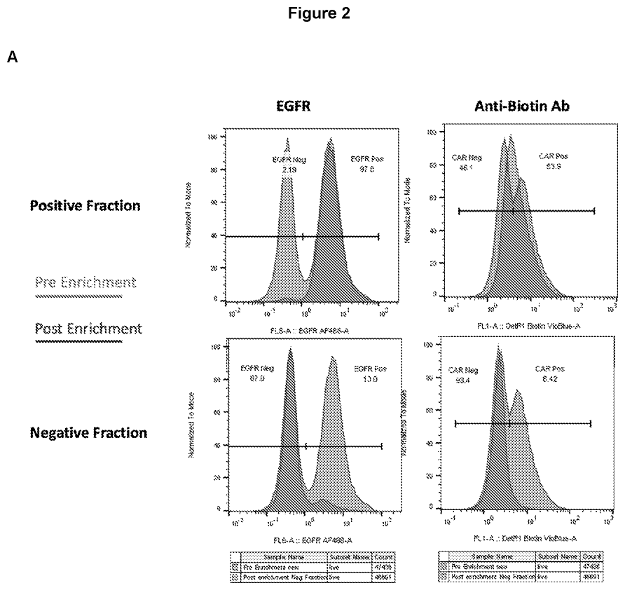

FIG. 2 shows the results of the enrichment protocol, pre- and post-enrichment, after 0 hr and 48 hr post-enrichment of “Donor 12” nfP2X₇ CAR T cells whereby tEGFR was used as the marker gene to identify the CAR expressing cells. Direct detection of nfP2X₇ CAR positive cells was done by indirect staining using the same LC-LC-biotinylated Fc attenuated fusion protein DetR1 or 2 and a secondary anti-biotin antibody. After a single round of positive selection, the purity of CAR expressing cells was >90%. 48 hours post enrichment (FIG. 2B) demonstrates the survival and purity was still >90%.

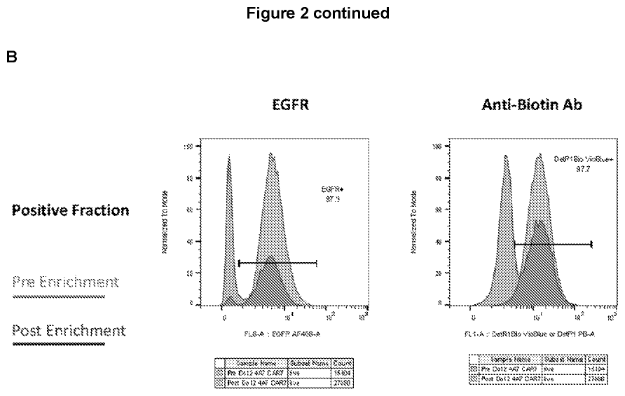

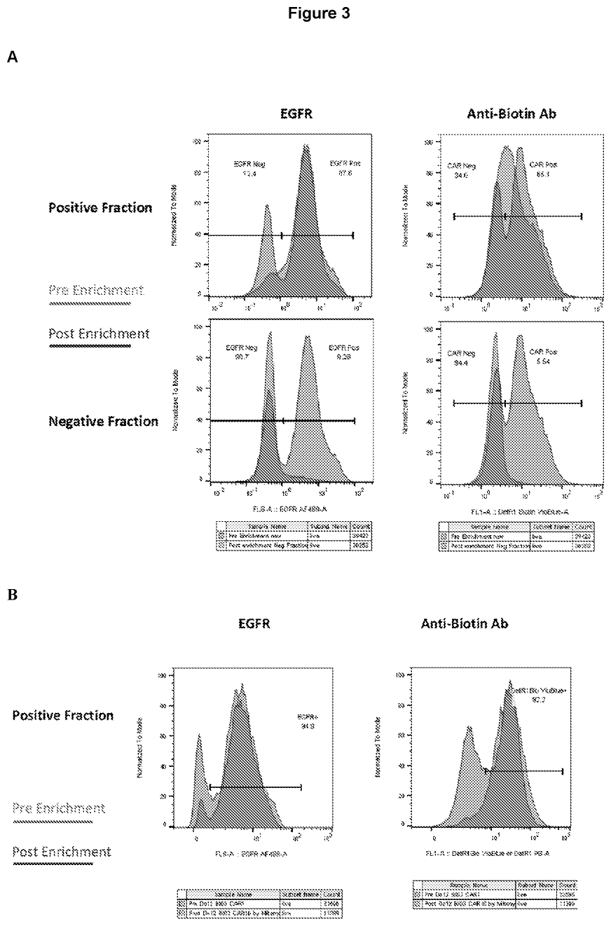

FIG. 3 shows the results of the enrichment protocol, pre- and post-enrichment, after 0 hr and 48 hr post-enrichment of “Donor 12” nfP2X₇ CAR T cells (using a CAR with an alternative nfP2X₇ receptor-antigen binding domain to the above paragraph, “CAR2”), whereby tEGFR was used as the marker gene to identify the CAR expressing cells. Direct detection of nfP2X₇ CAR positive cells was done by indirect staining using the same LC-LC-biotinylated Fc attenuated fusion protein DetR1 or 2 and a secondary anti-biotin antibody. After a single round of positive selection, the purity of CAR expressing cells was >90%. 48 hours post enrichment (FIG. 3B) demonstrates the survival and purity was still >90%.

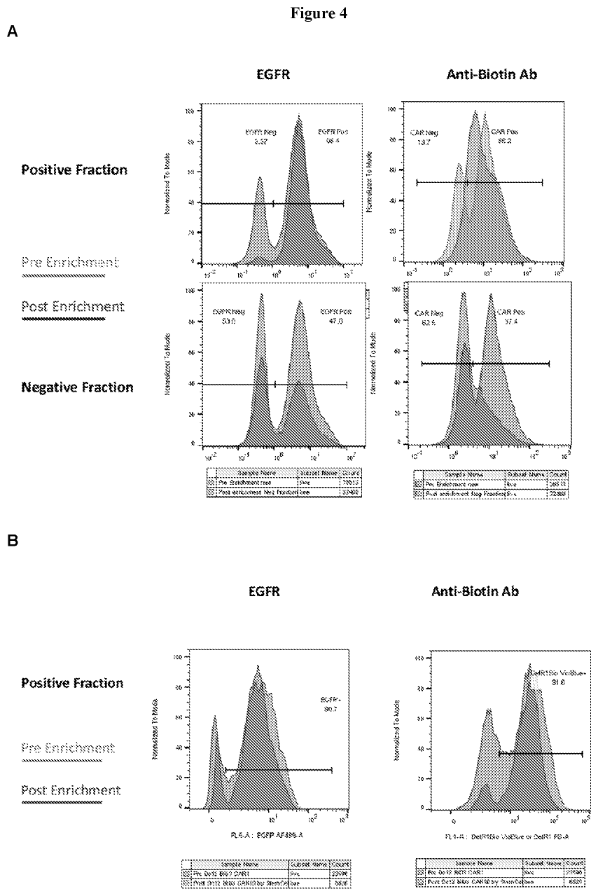

FIG. 4 shows the results of the enrichment protocol, pre- and post-enrichment using the EasySep technology as a comparison to the MACS technology in FIG. 3, after 0 hr and 48 hr post-enrichment of “Donor 12” nfP2X₇ CAR T cells, whereby tEGFR was used as the marker gene to identify the CAR expressing cells. Direct detection of nfP2X₇ CAR positive cells was done by indirect staining using the same LC-LC-biotinylated Fc attenuated fusion protein DetR1 or 2 and a secondary anti-biotin antibody. After a single round of positive selection, the purity of CAR expressing cells was >90%. 48 hours post enrichment (FIG. 4B) demonstrates the survival and purity was still >90%.

The below table shows a summary of the enrichments performed in FIG. 1-4.

| | | Post | Postive | Post | | |
|---|---|---|---|---|---|---|
| | | enrichment | Fraction | enrichment | Post | |
| | | Postive | purity | Negative | enrichment | |
| | Pre | Fraction | By EGFR | Fraction | In Total | Yield |
| Sample | (×10\hat{ }6) | (×10\hat{ }6) | (%) | (×10\hat{ }6) | (×10\hat{ }6) | % |
| Jurkat | 10 | 2.76 | 91% | 4.46 | 7.22 | 72.2% |
| UTD + CAR1 | | | | | | |
| Donor 12 | 10 | 3.11 | 97.8% | 3.14 | 6.25 | 62.5% |
| CAR2 | | | | | | |
| Donor 12 | 10 | 4.52 | 87.6% | 2.1 | 6.62 | 66.2% |
| CAR3 | | | | | | |
| using | | | | | | |
| Miltenyi | | | | | | |
| Column | | | | | | |
| Donor 12 | 10 | 2.26 | 96.4% | 2.68 | 4.94 | 49.4% |
| CAR3 | | | | | | |
| using Stem | | | | | | |
| cell magnet | | | | | | |

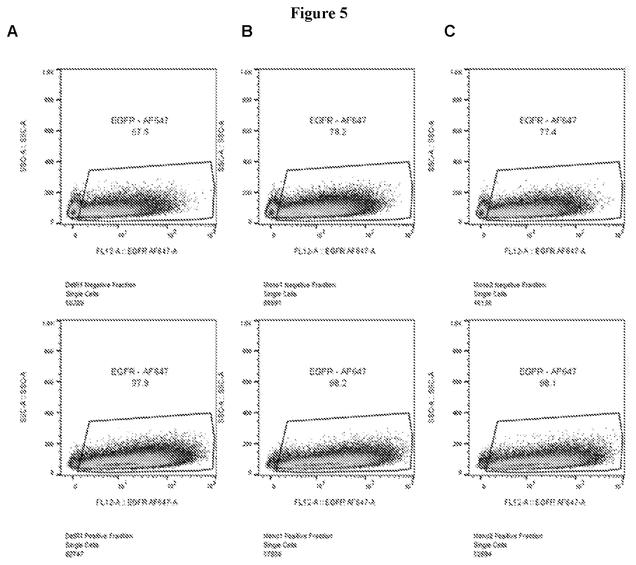

FIG. 5 shows the results of the enrichment protocol, pre- and post-enrichment, after 0 hr and 48 hr post-enrichment of “Donor 57” nfP2X₇ CAR T cells, whereby tEGFR was used as the marker gene to identify the CAR expressing cells. Direct detection of nfP2X₇ CAR positive cells was done by indirect staining using the same LC-LC-biotinylated Fc attenuated fusion protein DetR1 or 2 and a secondary anti-biotin antibody.

The enrichment was done by MACS technology. The data illustrates the enrichment with the dimeric, Fc-attenuated fusion protein DetR1 (SEQ ID NO: 149) compared to the monomeric, Fc-attenuated fusion protein DetR1 (SEQ ID NO: 145) and the monomeric, Fc-attenuated fusion protein DetR2 (SEQ ID NO: 146).

Conditions:
1. LC-LC-biotinylated dimeric DetR1 Fc attenuated
2. LC-LC-biotinylated monomeric DetR1 Fc attenuated
3. LC-LC-biotinylated monomeric DetR1 Fc attenuated

Pre-separation CAR expression was 55% via (DetR1) and 60% via tEGFR. A total of 1x10E⁷ T cells were labelled at a concentration of 2 μg/mL at RT for 10 min before separation was continued according to the manufacturer instructions for MACS separation (please read above) using MS columns.

After a single round of positive selection, the purity of CAR expressing cells was >90%. 48 hours post enrichment demonstrates the survival and purity was still >90%.

Cell count prior to and post separation (=YIELD) and EGFR expression shows CAR positive fraction, CAR negative fraction. The use of marker gene facilitates direct detection of the CAR.

The results are summarised in the below table:

| | | | | | Post- | |
|---|---|---|---|---|---|---|
| | | Post positive | Positive | Post negative | enrichment | |
| | Pre count | fraction | fraction by | fraction | in total | |
| Sample | (×10\hat{ }6) | (×10\hat{ }6) | purity (EGFR) | (×10\hat{ }6) | (×10\hat{ }6) | Yield |
| DetR1 | 10 | 3.5 | 97.9% | 3.2 | 3.42 | 62% |
| Attenuated | | | | | | |
| DetR1 | 10 | 1.87 | 98.2% | 5.4 | 1.53 | 28% |
| monomer | | | | | | |
| DetR2 | 10 | 1.86 | 98.1% | 5.54 | 1.69 | 30.7% |
| Monomer | | | | | | |

Although these data indicate that yields were higher using a dimeric form of fusion protein, in practice it would be preferable to utilise a monomeric protein to avoid activation of the immune cells being enriched. Moreover, the above results may be a reflection of superior biotin labelling using a dimeric protein compared to monomeric protein which provides for the increased yield.

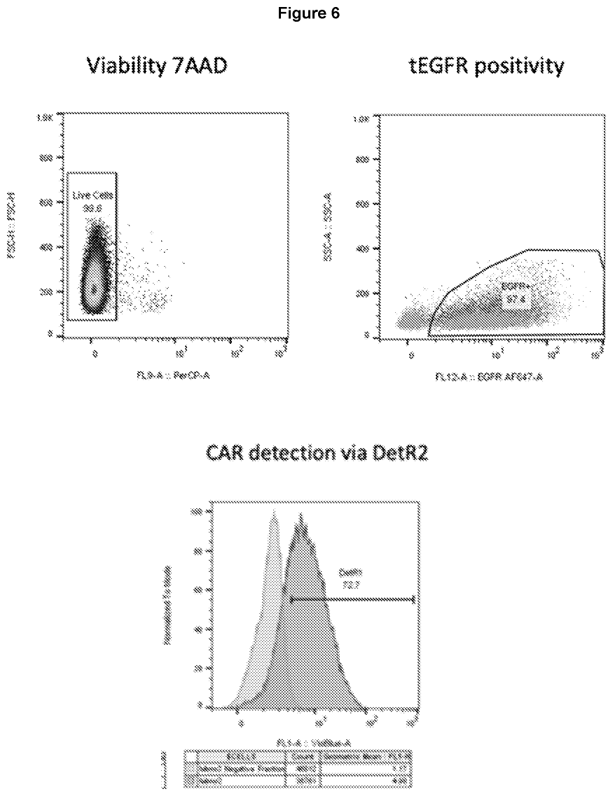

FIG. 6 shows purity and viability of the CAR T cells post separation measured by viability dye (7AAD, measured in the channel PerCP) and indirect CAR detection using the marker gene tEGFR (AF647 primary labelled anti-EGFR mAb cetuximab, measured in the APC channel). The direct detection of the CAR receptor shows still a partially occupied situation of the isolated CAR positive cells. T cells expressing anti-nfP2X₇ CAR were detected by staining of CAR T cells with biotinylated DetR2 (SEQ ID NO: 146) and then with a secondary anti-biotin antibody in Vioblue (130-113-857 Biotin Antibody, VioBlue®, Miltenyi Biotech).

### Example 2: Enrichment Using Monomeric Fusion Proteins Leads to Less Activation and Exhaustion Compared to Homodimeric Fusion Proteins

A similar series of enrichment experiments to those described in Example 1 were performed. Briefly, Jurkat cells and/or primary CD4+ T cells and CD8+ T cells (mixed at 1:1 ratio after enrichment) from a healthy volunteer donor (Donor 24) were stably transduced by lentivirus (3rd gen LV system) to express an an E200-targeted chimeric antigen receptor (CAR), wherein the CAR comprised an antigen binding domain for binding to the E200 epitope of the P2X₇ receptor.

CAR T cells were enriched using either a monomeric fusion protein or a dimeric fusion protein, each comprising a peptide moiety capable of being bound by the CAR or protein. The fusion proteins used in this experiment comprise the amino acid sequences of SEQ ID NOs: 158 (monomeric) and 149 (which is capable of forming homodimers in vitro).

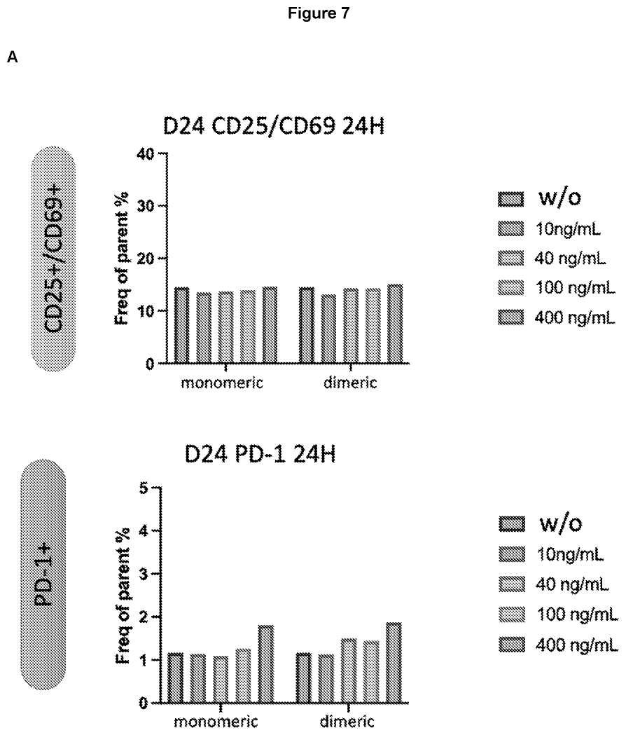

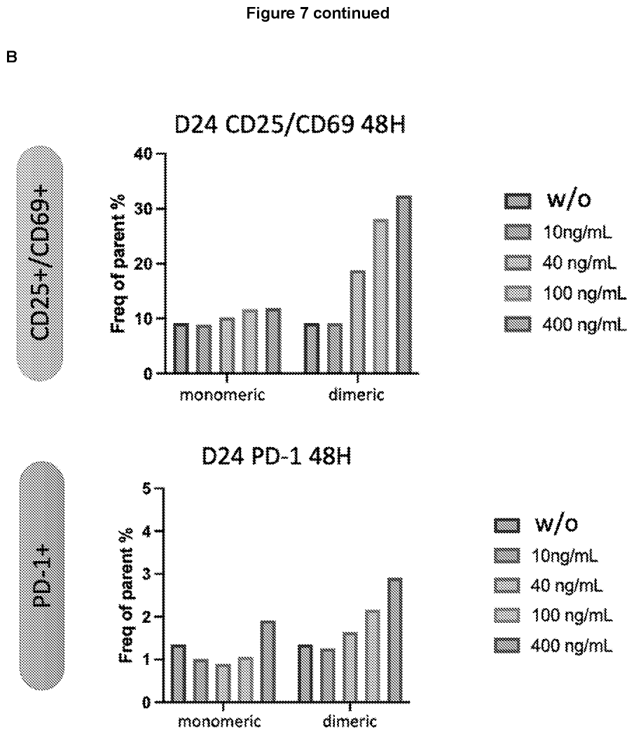

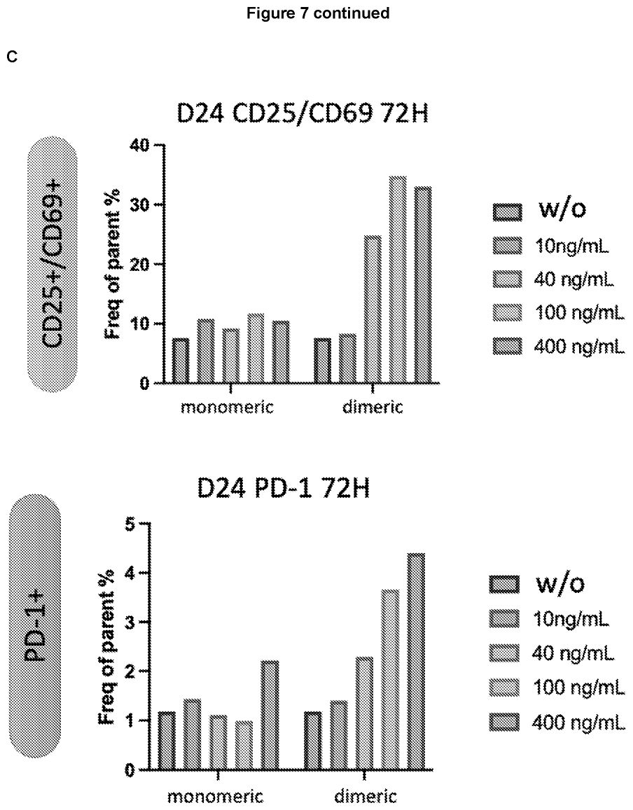

FIG. 7 shows the levels of CD25+/CD69+ expression (each measures of T cell activation) and the levels of PD-1 expression (a measure of T cell exhaustion), at 24 hours, 48 hours and 72 hours following enrichment with varying concentrations of the fusion proteins (10 ng/ml to 400 ng/ml).

The results show that enrichment using a monomeric fusion protein leads to significantly less T cell activation and significantly less T cell exhaustion, in a concentration dependent manner, compared to when using a dimeric fusion protein.

### Example 3: Efficiency, Viability and Yield Following CAR T Enrichment Using Biotin Beads and MS Columns

Enrichment of CAR T cells using MACS technology was performed according to “method 1” in Example 1. T cells were obtained from three healthy donors (donors 50, 53 and 71, D50, D53 and D71).

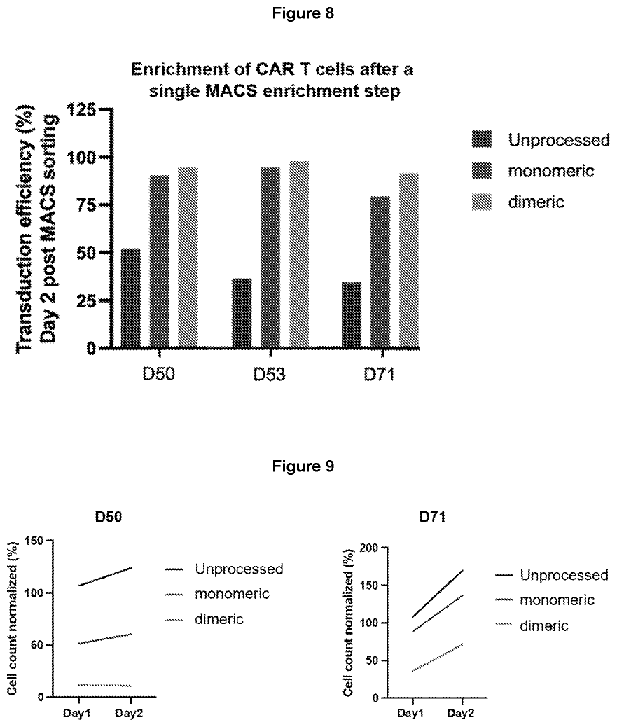

FIG. 8 shows that enrichment using monomeric or dimeric fusion proteins is approximately equivalent, indicating that there is no significant loss of enrichment potential using a monomeric fusion protein as compared to a dimeric fusion protein.

The results shown are indicative of two repeat experiments using T cells from donors 50 and 71.

FIG. 9 shows cell counts on Days 1 and 2 post MACS sorting, normalised to maximal cell count expected. The results demonstrate that there is a significantly higher cell count obtained when using a monomeric fusion protein for the enrichment process, compared to when using a homodimeric fusion protein. Results shown are for T cells obtained from two health donors (donor 50 and donor 71).

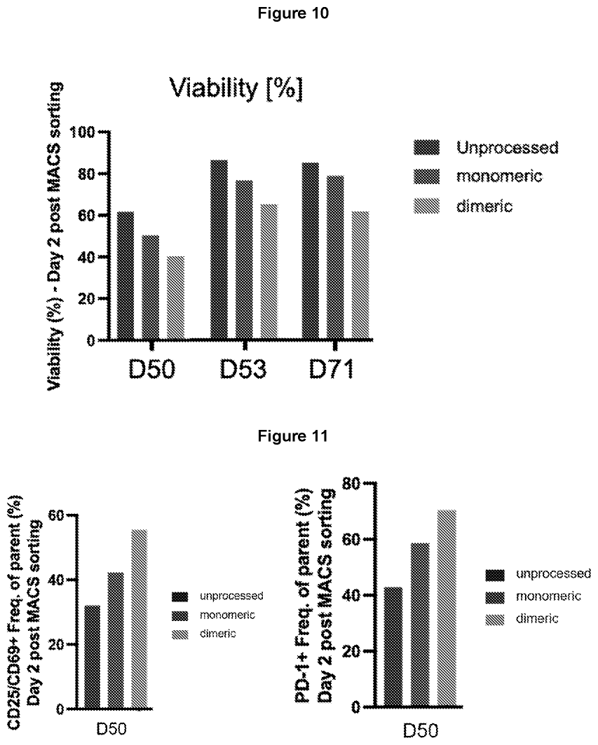

FIG. 10 shows the viability of cells 2 days following MACS sorting, as measured by the percentage of 7AAD negative cells in the cell population. The results show reduced overall cell viability when enrichment is performed using a dimeric fusion protein when compared to using a monomeric fusion protein. The results shown are indicative of two repeat experiments using T cells from donors 50 and 71.

FIG. 11 shows CD25+/CD69+ expression and PD-1 expression of the enriched CAR T cells, 2 days following MACS sorting.

The results show that cells enriched using a dimeric fusion protein have significantly higher levels of the markers of activation CD25 and CD69, compared to cells that are enriched using a monomeric fusion protein. Further, cells enriched using a dimeric fusion protein have significantly higher levels of the exhaustion marker PD-1 compared to cells enriched using a monomeric fusion protein.

Overall, the results show that enrichment of CAR T cells using a monomeric fusion protein (such as having the amino acid sequence of SEQ ID NO: 158), provides for:
a comparable level of enrichment of CAR positive T cells to the levels obtained when using a dimeric fusion protein
improved recovery of cells with higher cell count and higher cell viability compared to when enriching using a homodimeric fusion protein (eg comprising two copies of an E200-derived peptide)
less activation and less exhaustion of enriched cells compared to cells obtained using a dimeric fusion protein.

These results indicate that a superior cellular product is obtained using an enrichment method that includes use of a monomeric fusion protein as described herein, as compared to when using a homodimeric fusion protein that comprises two moieties (peptide sequences) capable of being bound by a CAR and which would likely lead to cross-activation of CAR T cells.

### Example 4: Enrichment Using Asymmetric Heterodimeric Molecules

Similar experiments are conducted using heterodimeric asymmetric molecules as described herein (eg such that the molecules comprise dimerisation between an E200 peptide-Fc fusion protein and a non-identical Fc region of an antibody; using KIH technology). The results similarly show that enriching CAR T cells using a heterodimeric asymmetric molecule comprising a single copy of the E200 peptide sequence, leads to significantly less T cell activation and significantly less T cell exhaustion, in a concentration dependent manner, compared to when using a homodimeric fusion protein that comprises two copies of the E200 peptide (eg wherein the dimer is a homodimer of E200-Fc fusion proteins). These results indicate that for the purposes of enriching CAR T cells it is preferable to use an asymmetric heterodimer molecule or monomeric fusion protein (eg comprising a single amino acid sequence capable of being recognised by the antigen binding domain of the CAR) in order to minimise unwanted activation and exhaustion of the CAR T cells in the patient.

It will be understood that the invention disclosed and defined in this specification extends to all alternative combinations of two or more of the individual features mentioned or evident from the text or drawings. All of these different combinations constitute various alternative aspects of the invention.

1. A fusion protein comprising:
(i) a dysfunctional P2X₇ receptor epitope moiety; and
(ii) an Fc region of an antibody.

2. The fusion protein of claim 1, wherein the dysfunctional P2X₇ receptor epitope moiety comprises the sequence of an epitope which is found on dysfunctional P2X₇ receptor but not on functional P2X₇ receptor.

3. The fusion protein of claim 1 or 2, wherein the amino acid sequence of the dysfunctional P2X₇ receptor epitope moiety comprises or consists at least of the amino acid sequence as set forth in SEQ ID NO: 7, preferably the amino acid sequence of SEQ ID NO: 14.

4. The fusion protein of any one of claims 1 to 3, wherein the amino acid sequence of the dysfunctional P2X₇ receptor epitope moiety comprises or consists of the sequence as set forth in SEQ ID NO: 9 or 122.

5. The fusion protein of any one of claims 1 to 3, wherein the amino acid sequence of dysfunctional P2X₇ receptor epitope moiety comprises an amino acid sequence as set forth in any of SEQ ID NOs: 7 to 69 or 122, or sequences at least 80%, at least 81%, at least 82%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical thereto, provided that the sequences comprise at least the sequence set forth in SEQ ID NO: 14 or 7.

6. The fusion protein of any one of claims 1 to 5, wherein the Fc region of an antibody is an Fc region of an IgG, IgA, IgD, IgE, or IgM.

7. The fusion protein of claim 6, wherein the Fc region is from an IgG antibody, preferably IgG1.

8. The fusion protein of any one of claims 1 to 7, wherein the Fc region of the fusion protein comprises the CH2 and CH3 domains of an antibody.

9. The fusion protein of any one of claim 1 to 8, wherein the Fc region of the fusion protein comprises one or more amino acid substitutions compared to naturally occurring Fc sequences, which prevent or reduce the ability of the Fc region to homodimerise.

10. The fusion protein of claim 9, wherein the amino acid substitutions comprise one or more substitutions of the cysteine residues so as to prevent the formation of disulphide bonds between Fc molecules, optionally wherein the cysteine residues of the Fc region are substituted to any of: glycine, serine, alanine, lysine and glutamic acid, preferably glycine or serine.

11. The fusion protein of any one of claims 1 to 10, wherein the Fc region comprises one or more amino acid substitutions for reducing affinity for the Fc receptor (FcR, including any of FcγRI, FcγRII and FcγRIII) and/or for reducing recruitment of complement C1q and/or for reducing affinity for FcRn.

12. The fusion protein of claim 11, wherein the one or more amino acid substitutions reduce the ability of the fusion protein to elicit antibody-dependent cell-mediated toxicity (ADCC).

13. The fusion protein of any one of claims 1 to 12, wherein the fusion protein has an affinity for FcR that is less than about 250 nM, preferably less than 500 nM, less than 1000 nM, most preferably less than 2000 nM.

14. The fusion protein of any one of claims 1 to 13, wherein the protein comprises a linker region, optionally a cleavable linker region, for joining the dysfunctional P2X₇ receptor epitope moiety and Fc region of an antibody.

15. The fusion protein of any one of claims 1 to 14, wherein the fusion protein comprises one or more modifications for enabling detection of the fusion protein including when the fusion protein is bound to a cell or to a binding partner.

16. The fusion protein of claim 15, wherein the one or more modifications enables capture of the fusion protein when bound to an immune cell expressing a receptor comprising an antigen binding domain for binding to dysfunctional P2X₇ receptor (such as a chimeric antigen receptor (CAR) for binding to dysfunctional P2X₇ receptor).

17. The fusion protein of claim 16, wherein the one or more modifications is selected from: a biotin moiety, fluorescein (FITC), a peptide tag (such as His, Myc, Flag and related tags) and a magnetic label.

18. An asymmetric heterodimeric molecule comprising a fusion protein of any one of claims 1 to 17.

19. Use of a fusion protein or molecule of any one of claims 1 to 18, for obtaining a population of immune cells, which are enriched for cells which express a receptor comprising an antigen binding domain for binding to dysfunctional P2X₇ receptor.

20. The use of claim 19, wherein the immune cells express a chimeric antigen receptor for binding dysfunctional P2X₇ receptor.

21. A method for obtaining a population of immune cells or for enriching for a population of cells expressing a receptor that comprises an antigen binding domain for binding to a tumour associated or tumour specific antigen on a cancer cell, the method comprising:
(i) providing a population of immune cells, preferably immune effector cells, wherein the cells have been subjected to transduction with a nucleic acid encoding a receptor comprising an antigen binding domain for binding to a tumour associated or tumour specific antigen on a cancer cell;
(ii) contacting the population of cells with a polypeptide, wherein the polypeptide comprises an epitope which is recognised by the antigen binding domain of the receptor, and wherein the polypeptide comprises a moiety for enabling capture of the polypeptide;
to thereby form a complex of the polypeptide and the cells;
(iii) isolating the complex from the population of cells,
thereby obtaining a population of immune cells expressing a receptor having an antigen binding domain for binding to a tumour associated or tumour specific antigen on a cancer cell.

22. A method of obtaining a population of immune cells or for enriching for a population of cells expressing a receptor that comprises an antigen binding domain for binding to a tumour associated or tumour specific antigen on a cancer cell, the method comprising:
(i) providing a mixed population of immune cells, preferably immune effector cells, wherein a subpopulation of the cells express receptor comprising an antigen binding domain for binding to a tumour associated or tumour specific antigen on a cancer cell;
(ii) contacting the mixed population of cells with a polypeptide, wherein the polypeptide comprises an epitope which is recognised by the antigen binding domain of the receptor, and wherein the polypeptide comprises a moiety for enabling capture of the polypeptide;
to thereby form a complex of the polypeptide and cells;
(iii) isolating the complex from the population of cells,
thereby obtaining a population of immune cells expressing a receptor having an antigen binding domain for binding to a tumour associated or tumour specific antigen on a cancer cell.

23. The method of claim 21 or 22, wherein the cells are immune cells which express a chimeric antigen receptor (CAR) for binding to a tumour associated or tumour specific antigen on a cancer cell and wherein the polypeptide comprises an epitope which is recognised by the CAR, thereby obtaining or enriching a population of immune cells expressing a CAR.

24. The method of any one of claims 21 to 23, wherein the immune cells express a receptor for binding to a tumour associated or tumour specific antigen selected from: dysfunctional (nf)P2X₇ receptor, mesothelin, EGFR, GPC3, MUC1, HER2, GD2, CEA, EpCAM, LeY, PCSA, CD19, CD20, Clec9a, CD276, PD-L1 and PD-L2.

25. A method for obtaining a population of immune cells or for enriching for a population of cells expressing a receptor that comprises an antigen binding domain for binding to dysfunctional P2X₇ receptor, the method comprising:
(i) providing a population of immune cells, preferably immune effector cells, wherein the cells have been subjected to transduction with a nucleic acid encoding a receptor comprising an antigen binding domain for binding to dysfunctional P2X₇ receptor;
(ii) contacting the population of cells with a polypeptide, wherein the polypeptide comprises an epitope of dysfunctional P2X₇ receptor which is recognised by the antigen binding domain of the receptor, and wherein the polypeptide comprises a moiety for enabling capture of the polypeptide;
to thereby form a complex of the polypeptide and the cells;
(iii) isolating the complex from the population of cells,
thereby obtaining a population of immune cells expressing a receptor having an antigen binding domain for binding to dysfunctional P2X₇ receptor.

26. A method for obtaining a population of immune cells or for enriching for a population of cells expressing a receptor that comprises an antigen binding domain for binding to dysfunctional P2X₇ receptor, the method comprising:
(i) providing a mixed population of immune cells, preferably immune effector cells, wherein a subpopulation of the cells express receptor comprising an antigen binding domain for binding to dysfunctional P2X₇ receptor;
(ii) contacting the mixed population of cells with a polypeptide, wherein the polypeptide comprises an epitope of dysfunctional P2X₇ receptor which is recognised by the antigen binding domain of the receptor, and wherein the polypeptide comprises a moiety for enabling capture of the polypeptide;
to thereby form a complex of the polypeptide and cells;
(iii) isolating the complex from the population of cells,
thereby obtaining a population of immune cells expressing a receptor having an antigen binding domain for binding to dysfunctional P2X₇ receptor.

27. The method of claims 25 or 26, wherein the cells to be obtained or enriched are immune cells which express a chimeric antigen receptor (CAR) for binding to dysfunctional P2X₇ receptor.

28. A method for obtaining a population of immune cells expressing a chimeric antigen receptor (CAR) for binding to dysfunctional P2X₇ receptor, the method comprising:
(i) providing a mixed population of immune cells, preferably immune effector cells, wherein the population comprises cells which have been transduced with a nucleic acid encoding a chimeric antigen receptor (CAR) for binding to dysfunctional P2X₇ receptor; or wherein the cells express a chimeric antigen receptor (CAR) for binding to dysfunctional P2X₇ receptor;
(ii) contacting the population of cells with a polypeptide, wherein the polypeptide comprises an epitope of dysfunctional P2X₇ receptor which is recognised by the CAR, and wherein the polypeptide comprises a moiety for enabling capture of the polypeptide;
to thereby form a complex of the polypeptide and the cells;
(iii) isolating the complex from the population,
thereby obtaining a population of immune cells expressing a chimeric antigen receptor (CAR) for binding to dysfunctional P2X₇ receptor.

29. A method for enriching a population of immune cells expressing a chimeric antigen receptor comprising an antigen binding domain for binding to dysfunctional P2X₇ receptor, the method comprising:
(i) providing a population of immune cells, preferably immune effector cells, wherein the cells have been subjected to transduction with a nucleic acid encoding a chimeric antigen receptor (CAR) for binding to dysfunctional P2X₇ receptor;
(ii) contacting the population of cells with a polypeptide, wherein the polypeptide comprises an epitope of dysfunctional P2X₇ receptor which is recognised by the CAR, and wherein the polypeptide comprises a moiety for enabling capture of the polypeptide,
to thereby form a complex of the polypeptide and the cells;
(iii) isolating the complex of cells expressing a chimeric antigen receptor (CAR) for binding to dysfunctional P2X₇ receptor bound to fusion protein, from the mixed population,
thereby enriching a population of immune cells expressing a chimeric antigen receptor (CAR) for binding to dysfunctional P2X₇ receptor.

30. The method of any one of claims 21 to 29 wherein the polypeptide comprises a first portion comprising an epitope of a tumour associated or tumour specific antigen (such as dysfunctional P2X₇ receptor) joined to a further amino acid sequence for facilitating the solubility and stability of the first portion.

31. The method of claim 30, wherein the further amino acid sequence joined to the epitope of the tumour associated or tumour specific antigen, comprises a linker or hinge region.

32. The method of claim 31, wherein the linker comprises a cleavable sequence.

33. The method of claim 31 or 32, wherein the polypeptide is in the form of a fusion protein comprising an epitope of dysfunctional P2X₇ receptor joined to a further amino acid sequence optionally wherein the further sequence is selected from serum albumin, transferrin, a carboxy-terminal peptide of chorionic gonadotropin (CG) p chain, a non-exact repeat peptide sequence, a polypeptide sequence composed of proline-alanine-serine polymer, an elastin-like peptide (ELP) repeat sequence), a homopolymer of glycine residues or a gelatin-like protein.

34. The method of any one of claims 21 to 29, wherein the polypeptide is in the form of a conjugate comprising a carbohydrate, a lipid, a liposome, a peptide, and an aptamer conjugated to the amino acid sequence comprising the epitope of dysfunctional P2X₇ receptor.

35. The method of any one of claims 21 to 34, wherein the moiety for enabling capture of the polypeptide is a detectable moiety selected from: a biotin moiety, fluorescein (FITC), a peptide tag (such as His, Myc, Flag and related tags) and a magnetic label.

36. The method of any one of claims 20 to 35, wherein the polypeptide is labelled with a biotin moiety, and the method further comprises the step of contacting the cells (after step ii), with an anti-biotin antigen binding protein, preferably wherein the anti-biotin antigen binding protein comprises one or more moieties for enabling capture thereof.

37. The method of claim 36 wherein the one or more moieties for enabling capture of the anti-biotin antigen binding protein comprises iron oxide particles (micro- or macrobeads).

38. The method of any one of claims 21 to 35 wherein the polypeptide comprises a magnetic label, and the step of isolating comprises i) applying a magnetic field to the population of cells; ii) removing or discarding the cells that are not attracted to the magnetic field, iii) removal of the magnetic field to thereby provide a population of immune cells expressing the receptor.

39. The method of any one of claims 21 to 38, wherein the method further comprises the steps of expanding the isolated immune cells.

40. The method of any one of claims 21 to 39, wherein the method further comprises the step of administering the isolated cells to a subject requiring treatment for cancer.

41. The method of any one of claims 21 to 40, wherein the population of immune cells is a population of effector immune cells, such as T cells, NK cells or NKT cells.

42. The method of claim 41, wherein the immune cells are T cells.

43. The method of claim 41 or 42, wherein the cells are derived from stem cells, optionally wherein the stem cells are induced pluripotent stem cells (iPSCs) or embryonic stem cells.

44. The method of claim 41 or 43, wherein the cells are derived from a subject requiring treatment for cancer.

45. The method of claims 41 or 42, wherein the cells are obtained or derived from an allogeneic donor who does not require treatment.

46. A composition comprising a population of immune cells expressing a chimeric antigen receptor (CAR) for binding to a tumour associate or tumour specific antigen, wherein the population of cells is obtained by the method of any one of claims 21 to 45.

47. The composition of claim 46, wherein the composition comprises greater than 30%, 40%, 50%, 60%, 70%, 80%, 85%, 90%, 91%, 92%, 93%, 94%, 95%, 96%, 97%, 98 or 99% of cells that express a chimeric antigen receptor (CAR) for binding to a tumour associate or tumour specific antigen.

48. The composition of claim 46 or 47, wherein the composition is for use in the treatment of cancer in a subject in need thereof.

49. A method of treating cancer in a subject in need thereof, the method comprising administering to the subject, a composition according to claim 46 or 47.

50. Use of the composition of claim 46 or 47, in the manufacture of a medicament for treating cancer in a subject.

51. A kit for use in a method of any one of claims 21 to 45, the kit comprising:
a fusion protein of any one of claims 1 to 17, a heterodimer of claim 18, or a polypeptide comprising an epitope of a tumour associate or tumour specific antigen and a moiety for enabling capture of the polypeptide;
optionally, one or more reagents for enabling isolation of the fusion protein and complexes thereof.

52. The fusion protein of any one of claims 1 to 17, or the heterodimer of claim 18, comprising the amino acid sequence as set forth in any one of SEQ ID NOs: 145 to 158 or 160 and 161.

53. The method of any one of claims 21 to 29, wherein the polypeptide comprises the amino acid sequence of a fusion protein of any one of claims 1 to 17.

54. The method of any one of claims 21 to 45, wherein the polypeptide comprises the amino acid sequence as set forth in any one of SEQ ID NOs: 145 to 158 or 160 or 161.

55. The method of any one of claims 21 to 45, wherein the polypeptide comprises an asymmetric heterodimer of claim 18

56. The method of claim 55, wherein the asymmetric heterodimer comprises a fusion protein comprising the amino acid sequence of any one of SEQ ID NOs: 157, 157, 160 or 161.


SEQUENCE LISTING (162 sequences)

SEQ ID NO: 1 - protein, 595 aa, synthetic construct
MPACCSCSDVFQYETNKVTRIQSMNYGTIKWFFHVIIFSYVCFALVSDKLYQRKEPVISSVHTKVKGIAEVKEEIVENGVKKLVHSVFDTADYTFPLQGNSFFVMTNFLKTEGQEQRLCPEYPTRRTLCSSDRGCKKGWMDPQSKGIQTGRCVVYEGNQKTCEVSAWCPIEAVEEAPRPALLNSAENFTVLIKNNIDFPGHNYTTRNILPGLNITCTFHKTQNPQCPIFRLGDIFRETGDNFSDVAIQGGIMGIEIYWDCNLDRWFHHCRPKYSFRRLDDKTTNVSLYPGYNFRYAKYYKENNVEKRTLIKVFGIRFDILVFGTGGKFDIIQLVVYIGSTLSYFGLAAVFIDFLIDTYSSNCCRSHIYPWCKCCQPCVVNEYYYRKKCESIVEPKPTLKYVSFVDESHIRMVNQQLLGRSLQDVKGQEVPRPAMDFTDLSRLPLALHDTPPIPGQPEEIQLLRKEATPRSRDSPVWCQCGSCLPSQLPESHRCLEELCCRKKPGACITTSELFRKLVLSRHVLQFLLLYQEPLLALDVDSTNSRLRHCAYRCYATWRFGSQDMADFAILPSCCRWRIRKEFPKSEGQYSGFKSPY

SEQ ID NO: 2 - protein, 17 aa, synthetic construct
GHNYTTRNILPGLNITC

SEQ ID NO: 3 - protein, 16 aa, synthetic construct
GHNYTTRNILPGLNIT

SEQ ID NO: 4 - protein, 15 aa, synthetic construct
KYYKENNVEKRTLIK

SEQ ID NO: 5 - protein, 17 aa, synthetic construct
KYYKENNVEKRTLIKVF

SEQ ID NO: 6 - protein, 25 aa, synthetic construct
GHNYTTRNILPGAGAKYYKENNVEK

SEQ ID NO: 7 - protein, 17 aa, synthetic construct
GHNYTTRNILPGLNITS

SEQ ID NO: 8 - protein, 21 aa, synthetic construct
GHNYTTRNILPGLNITSTFHK

SEQ ID NO: 9 - protein, 22 aa, synthetic construct
GHNYTTRNILPGLNITSTFHKT

SEQ ID NO: 10 - protein, 23 aa, synthetic construct
GHNYTTRNILPGLNITSTFHKTC

SEQ ID NO: 11 - protein, 27 aa, synthetic construct
GHNYTTRNILPGLNITSTFHKTSGSGK

SEQ ID NO: 12 - protein, 23 aa, synthetic construct
GHNYTTRNILPGLNITSTFHKTS

SEQ ID NO: 13 - protein, 16 aa, synthetic construct
DFPGHNYTTRNILPGC

SEQ ID NO: 14 - protein, 11 aa, synthetic construct
NYTTRNILPGL

SEQ ID NO: 15 - protein, 22 aa, synthetic construct
GHNYTTRNILPGLNITSGGGGS

SEQ ID NO: 16 - protein, 27 aa, synthetic construct
GHNYTTRNILPGLNITSGGGGSGGGGS

SEQ ID NO: 17 - protein, 32 aa, synthetic construct
GHNYTTRNILPGLNITSGGGGSGGGGSGGGGS

SEQ ID NO: 18 - protein, 24 aa, synthetic construct
GHNYTTRNILPGLNITSTFHKTGS

SEQ ID NO: 19 - protein, 22 aa, synthetic construct
GHNYTTRNILPGLNITSTFHGS

SEQ ID NO: 20 - protein, 19 aa, synthetic construct
GHNYTTRNILPGLNITSGS

SEQ ID NO: 21 - protein, 22 aa, synthetic construct
DFPGHNYTTRNILPGLNITSGS

SEQ ID NO: 22 - protein, 25 aa, synthetic construct
DFPGHNYTTRNILPGLNITSGGGGS

SEQ ID NO: 23 - protein, 30 aa, synthetic construct
DFPGHNYTTRNILPGLNITSGGGGSGGGGS

SEQ ID NO: 24 - protein, 35 aa, synthetic construct
DFPGHNYTTRNILPGLNITSGGGGSGGGGSGGGGS

SEQ ID NO: 25 - protein, 30 aa, synthetic construct
DFPGHNYTTRNILPGLNITSTFHKTSGSGK

SEQ ID NO: 26 - protein, 32 aa, synthetic construct
DFPGHNYTTRNILPGLNITSTFHKTSGSGKGS

SEQ ID NO: 27 - protein, 35 aa, synthetic construct
DFPGHNYTTRNILPGLNITSTFHKTSGSGKGGGGS

SEQ ID NO: 28 - protein, 28 aa, synthetic construct
DFPGHNYTTRNILPGLNITSTFHGGGGS

SEQ ID NO: 29 - protein, 25 aa, synthetic construct
GHNYTTRNILPGLNITSTFHGGGGS

SEQ ID NO: 30 - protein, 30 aa, synthetic construct
DFPGHNYTTRNILPGLNITSTFHKTGGGGS

SEQ ID NO: 31 - protein, 27 aa, synthetic construct
GHNYTTRNILPGLNITSTFHKTGGGGS

SEQ ID NO: 32 - protein, 27 aa, synthetic construct
GHNYTTRNILPGLNITSEPKSSDKTHT

SEQ ID NO: 33 - protein, 29 aa, synthetic construct
GHNYTTRNILPGLNITSGSEPKSSDKTHT

SEQ ID NO: 34 - protein, 32 aa, synthetic construct
GHNYTTRNILPGLNITSGGGGSEPKSSDKTHT

SEQ ID NO: 35 - protein, 37 aa, synthetic construct
GHNYTTRNILPGLNITSTFHKTSGSGKEPKSSDKTHT

SEQ ID NO: 36 - protein, 39 aa, synthetic construct
GHNYTTRNILPGLNITSTFHKTSGSGKGSEPKSSDKTHT

SEQ ID NO: 37 - protein, 42 aa, synthetic construct
GHNYTTRNILPGLNITSTFHKTSGSGKGGGGSEPKSSDKTHT

SEQ ID NO: 38 - protein, 29 aa, synthetic construct
GHNYTTRNILPGLNITSEPKSSDKTHTGS

SEQ ID NO: 39 - protein, 31 aa, synthetic construct
GHNYTTRNILPGLNITSGSEPKSSDKTHTGS

SEQ ID NO: 40 - protein, 34 aa, synthetic construct
GHNYTTRNILPGLNITSGGGGSEPKSSDKTHTGS

SEQ ID NO: 41 - protein, 39 aa, synthetic construct
GHNYTTRNILPGLNITSTFHKTSGSGKEPKSSDKTHTGS

SEQ ID NO: 42 - protein, 41 aa, synthetic construct
GHNYTTRNILPGLNITSTFHKTSGSGKGSEPKSSDKTHTGS

SEQ ID NO: 43 - protein, 44 aa, synthetic construct
GHNYTTRNILPGLNITSTFHKTSGSGKGGGGSEPKSSDKTHTGS

SEQ ID NO: 44 - protein, 32 aa, synthetic construct
GHNYTTRNILPGLNITSEPKSSDKTHTGGGGS

SEQ ID NO: 45 - protein, 34 aa, synthetic construct
GHNYTTRNILPGLNITSGSEPKSSDKTHTGGGGS

SEQ ID NO: 46 - protein, 37 aa, synthetic construct
GHNYTTRNILPGLNITSGGGGSEPKSSDKTHTGGGGS

SEQ ID NO: 47 - protein, 42 aa, synthetic construct
GHNYTTRNILPGLNITSTFHKTSGSGKEPKSSDKTHTGGGGS

SEQ ID NO: 48 - protein, 44 aa, synthetic construct
GHNYTTRNILPGLNITSTFHKTSGSGKGSEPKSSDKTHTGGGGS

SEQ ID NO: 49 - protein, 47 aa, synthetic construct
GHNYTTRNILPGLNITSTFHKTSGSGKGGGGSEPKSSDKTHTGGGGS

SEQ ID NO: 50 - protein, 30 aa, synthetic construct
DFPGHNYTTRNILPGLNITSEPKSSDKTHT

SEQ ID NO: 51 - protein, 32 aa, synthetic construct
DFPGHNYTTRNILPGLNITSGSEPKSSDKTHT

SEQ ID NO: 52 - protein, 35 aa, synthetic construct
DFPGHNYTTRNILPGLNITSGGGGSEPKSSDKTHT

SEQ ID NO: 53 - protein, 40 aa, synthetic construct
DFPGHNYTTRNILPGLNITSTFHKTSGSGKEPKSSDKTHT

SEQ ID NO: 54 - protein, 42 aa, synthetic construct
DFPGHNYTTRNILPGLNITSTFHKTSGSGKGSEPKSSDKTHT

SEQ ID NO: 55 - protein, 45 aa, synthetic construct
DFPGHNYTTRNILPGLNITSTFHKTSGSGKGGGGSEPKSSDKTHT

SEQ ID NO: 56 - protein, 32 aa, synthetic construct
DFPGHNYTTRNILPGLNITSEPKSSDKTHTGS

SEQ ID NO: 57 - protein, 34 aa, synthetic construct
DFPGHNYTTRNILPGLNITSGSEPKSSDKTHTGS

SEQ ID NO: 58 - protein, 37 aa, synthetic construct
DFPGHNYTTRNILPGLNITSGGGGSEPKSSDKTHTGS

SEQ ID NO: 59 - protein, 42 aa, synthetic construct
DFPGHNYTTRNILPGLNITSTFHKTSGSGKEPKSSDKTHTGS

SEQ ID NO: 60 - protein, 44 aa, synthetic construct
DFPGHNYTTRNILPGLNITSTFHKTSGSGKGSEPKSSDKTHTGS

SEQ ID NO: 61 - protein, 47 aa, synthetic construct
DFPGHNYTTRNILPGLNITSTFHKTSGSGKGGGGSEPKSSDKTHTGS

SEQ ID NO: 62 - protein, 35 aa, synthetic construct
DFPGHNYTTRNILPGLNITSEPKSSDKTHTGGGGS

SEQ ID NO: 63 - protein, 37 aa, synthetic construct
DFPGHNYTTRNILPGLNITSGSEPKSSDKTHTGGGGS

SEQ ID NO: 64 - protein, 40 aa, synthetic construct
DFPGHNYTTRNILPGLNITSGGGGSEPKSSDKTHTGGGGS

SEQ ID NO: 65 - protein, 45 aa, synthetic construct
DFPGHNYTTRNILPGLNITSTFHKTSGSGKEPKSSDKTHTGGGGS

SEQ ID NO: 66 - protein, 47 aa, synthetic construct
DFPGHNYTTRNILPGLNITSTFHKTSGSGKGSEPKSSDKTHTGGGGS

SEQ ID NO: 67 - protein, 50 aa, synthetic construct
DFPGHNYTTRNILPGLNITSTFHKTSGSGKGGGGSEPKSSDKTHTGGGGS

SEQ ID NO: 68 - protein, 40 aa, synthetic construct
DKTHTSPPSPAPELLGGGGSDFPGHNYTTRNILPGLNITS

SEQ ID NO: 69 - protein, 50 aa, synthetic construct
GGGGSEPKSSDKTHTSPPSPAPELLGGGGSDFPGHNYTTRNILPGLNITS

SEQ ID NO: 70 - protein, 5 aa, synthetic construct
GGGGS

SEQ ID NO: 71 - protein, 10 aa, synthetic construct
EPKSSDKTHT

SEQ ID NO: 72 - protein, 22 aa, synthetic construct
GSEPKSSDKTHTSPPSPAPELL

SEQ ID NO: 73 - protein, 30 aa, synthetic construct
GGGGSEPKSSDKTHTSPPSPAPELLGGGGS

SEQ ID NO: 74 - protein, 12 aa, synthetic construct
EPKSSDKTHTGS

SEQ ID NO: 75 - protein, 15 aa, synthetic construct
EPKSSDKTHTGGGGS

SEQ ID NO: 76 - protein, 15 aa, synthetic construct
EPKSSDKTHTSPPSP

SEQ ID NO: 77 - protein, 13 aa, synthetic construct
EPKSSDKTHTSPP

SEQ ID NO: 78 - protein, 14 aa, synthetic construct
EPKSSDKTHTSPPP

SEQ ID NO: 79 - protein, 10 aa, synthetic construct
EPKSSDKTHT

SEQ ID NO: 80 - protein, 10 aa, synthetic construct
DKTHTSPPSP

SEQ ID NO: 81 - protein, 8 aa, synthetic construct
DKTHTSPP

SEQ ID NO: 82 - protein, 12 aa, synthetic construct
EPKSDKTHTPPP

SEQ ID NO: 83 - protein, 14 aa, synthetic construct
EPKSDKTHTSPPSP

SEQ ID NO: 84 - protein, 15 aa, synthetic construct
EPKSGDKTHTGPPGP

SEQ ID NO: 85 - protein, 13 aa, synthetic construct
EPKSGDKTHTGPP

SEQ ID NO: 86 - protein, 14 aa, synthetic construct
EPKSGDKTHTGPPP

SEQ ID NO: 87 - protein, 10 aa, synthetic construct
EPKSGDKTHT

SEQ ID NO: 88 - protein, 10 aa, synthetic construct
DKTHTGPPGP

SEQ ID NO: 89 - protein, 8 aa, synthetic construct
DKTHTGPP

SEQ ID NO: 90 - protein, 14 aa, synthetic construct
EPKSDKTHTGPPGP

SEQ ID NO: 91 - protein, 15 aa, synthetic construct
EPKSGDKTHTSPPSP

SEQ ID NO: 92 - protein, 15 aa, synthetic construct
EPKSGDKTHTGPPSP

SEQ ID NO: 93 - protein, 15 aa, synthetic construct
EPKSSDKTHTGPPGP

SEQ ID NO: 94 - protein, 15 aa, synthetic construct
EPKSSDKTHTGPPSP

SEQ ID NO: 95 - protein, 13 aa, synthetic construct
EPKSSDKTHTGPP

SEQ ID NO: 96 - protein, 13 aa, synthetic construct
EPKSGDKTHTSPP

SEQ ID NO: 97 - protein, 15 aa, synthetic construct
EPKSTDKTHTTPPTP

SEQ ID NO: 98 - protein, 13 aa, synthetic construct
EPKSTDKTHTTPP

SEQ ID NO: 99 - protein, 14 aa, synthetic construct
EPKSTDKTHTTPPP

SEQ ID NO: 100 - protein, 10 aa, synthetic construct
EPKSTDKTHT

SEQ ID NO: 101 - protein, 10 aa, synthetic construct
DKTHTTPPTP

SEQ ID NO: 102 - protein, 8 aa, synthetic construct
DKTHTTPP

SEQ ID NO: 103 - protein, 14 aa, synthetic construct
EPKSDKTHTTPPTP

SEQ ID NO: 104 - protein, 15 aa, synthetic construct
EPKSSDKTHTTPPTP

SEQ ID NO: 105 - protein, 15 aa, synthetic construct
EPKSSDKTHTSPPTP

SEQ ID NO: 106 - protein, 15 aa, synthetic construct
EPKSADKTHTLPPMP

SEQ ID NO: 107 - protein, 15 aa, synthetic construct
EPKSVDKTHTLPPIP

SEQ ID NO: 108 - protein, 15 aa, synthetic construct
EPKSLDKTHTAPPAP

SEQ ID NO: 109 - protein, 13 aa, synthetic construct
EPKSVDKTHTAPP

SEQ ID NO: 110 - protein, 13 aa, synthetic construct
EPKSIDKTHTLPP

SEQ ID NO: 111 - protein, 10 aa, synthetic construct
DKTHTAPPLP

SEQ ID NO: 112 - protein, 10 aa, synthetic construct
DKTHTVPPLP

SEQ ID NO: 113 - protein, 12 aa, synthetic construct
EPKSSDKTHTSP

SEQ ID NO: 114 - protein, 17 aa, synthetic construct
EPKSCDKTHTCPPCPAP

SEQ ID NO: 115 - protein, 12 aa, synthetic construct
ERKCCVECPPCP

SEQ ID NO: 116 - protein, 12 aa, synthetic construct
ERKXXVEXPPXP

SEQ ID NO: 117 - protein, 10 aa, synthetic construct
ERKXXVEXPP

SEQ ID NO: 118 - protein, 7 aa, synthetic construct
VEXPPXP

SEQ ID NO: 119 - protein, 17 aa, synthetic construct
ELKTPLGDTTHTCPRCP

SEQ ID NO: 120 - protein, 17 aa, synthetic construct
ELKTPLGDTTHTXPRXP

SEQ ID NO: 121 - protein, 15 aa, synthetic construct
ELKTPLGDTTHTXPR

SEQ ID NO: 122 - protein, 25 aa, synthetic construct
DFPGHNYTTRNILPGLNITSTFHKT

SEQ ID NO: 123 - protein, 12 aa, synthetic construct
ELKTPLGDTTHT

SEQ ID NO: 124 - protein, 15 aa, synthetic construct
EPKSCDTPPPCPRCP

SEQ ID NO: 125 - protein, 15 aa, synthetic construct
EPKSXDTPPPXPRXP

SEQ ID NO: 126 - protein, 13 aa, synthetic construct
EPKSXDTPPPXPR

SEQ ID NO: 127 - protein, 10 aa, synthetic construct
EPKSXDTPPP

SEQ ID NO: 128 - protein, 10 aa, synthetic construct
DTPPPXPRXP

SEQ ID NO: 129 - protein, 12 aa, synthetic construct
ESKYGPPCPSCP

SEQ ID NO: 130 - protein, 12 aa, synthetic construct
EXKYGPPCPXCP

SEQ ID NO: 131 - protein, 9 aa, synthetic construct
EXKYGPPCP

SEQ ID NO: 132 - protein, 10 aa, synthetic construct
KYGPPCPXCP

SEQ ID NO: 133 - protein, 12 aa, synthetic construct
EPKSCDKTHTCP

SEQ ID NO: 134 - protein, 12 aa, synthetic construct
EPKSCDTPPPCP

SEQ ID NO: 135 - protein, 12 aa, synthetic construct
ESKYGPPSPSSP

SEQ ID NO: 136 - protein, 12 aa, synthetic construct
ERKSSVESPPSP

SEQ ID NO: 137 - protein, 12 aa, synthetic construct
EPKSSDTPPPSP

SEQ ID NO: 138 - protein, 5 aa, synthetic construct
TFHKT

SEQ ID NO: 139 - skipped

SEQ ID NO: 140 - protein, 20 aa, synthetic construct
DFPGHNYTTRNILPGLNITS

SEQ ID NO: 141 - protein, 17 aa, synthetic construct
EPKSCDKTHTSPPSPAP

SEQ ID NO: 142 - protein, 17 aa, synthetic construct
EPKSSDKTHTSPPSPAP

SEQ ID NO: 143 - protein, 5 aa, synthetic construct
GGGGS

SEQ ID NO: 144 - protein, 11 aa, synthetic construct
LEVLFQGPVRR

SEQ ID NO: 145 - protein, 266 aa, synthetic construct
DFPGHNYTTRNILPGLNITSTFHKTSGSGKGGGGSEPKSCDKTHTSPPSPAPPVAGPSVFLFPPKPKDTLMISRTPEVTCVVVGVSHEDPEVKFNWYVDGVEVHNAKTKPREEQYQSTYRVVSVLTVLHQDWLNGKEYKCKVSNKQLPSPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - protein, 263 aa, synthetic construct
GHNYTTRNILPGLNITSTFHKTSGSGKGGGGSEPKSCDKTHTSPPSPAPPVAGPSVFLFPPKPKDTLMISRTPEVTCVVVGVSHEDPEVKFNWYVDGVEVHNAKTKPREEQYQSTYRVVSVLTVLHQDWLNGKEYKCKVSNKQLPSPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 267 aa, synthetic construct
DFPGHNYTTRNILPGLNITSTFHKTSGSGKGGGGSEPKSSDKTHTSPPSPAPEAARGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - protein, 264 aa, synthetic construct
GHNYTTRNILPGLNITSTFHKTSGSGKGGGGSEPKSSDKTHTSPPSPAPEAARGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 266 aa, synthetic construct
DFPGHNYTTRNILPGLNITSTFHKTSGSGKGGGGSEPKSCDKTHTCPPCPAPPVAGPSVFLFPPKPKDTLMISRTPEVTCVVVGVSHEDPEVKFNWYVDGVEVHNAKTKPREEQYQSTYRVVSVLTVLHQDWLNGKEYKCKVSNKQLPSPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 263 aa, synthetic construct
GHNYTTRNILPGLNITSTFHKTSGSGKGGGGSEPKSCDKTHTCPPCPAPPVAGPSVFLFPPKPKDTLMISRTPEVTCVVVGVSHEDPEVKFNWYVDGVEVHNAKTKPREEQYQSTYRVVSVLTVLHQDWLNGKEYKCKVSNKQLPSPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 267 aa, synthetic construct
DFPGHNYTTRNILPGLNITSTFHKTSGSGKGGGGSEPKSSDKTHTCPPCPAPEAARGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 264 aa, synthetic construct
GHNYTTRNILPGLNITSTFHKTSGSGKGGGGSEPKSSDKTHTCPPCPAPEAARGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 277 aa, synthetic construct
DFPGHNYTTRNILPGLNITSTFHKTSGSGKGGGGSLEVLFQGPVRREPKSSDKTHTSPPSPAPPVAGPSVFLFPPKPKDTLMISRTPEVTCVVVGVSHEDPEVKFNWYVDGVEVHNAKTKPREEQYQSTYRVVSVLTVLHQDWLNGKEYKCKVSNKQLPSPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 154 - protein, 277 aa, synthetic construct
DFPGHNYTTRNILPGLNITSTFHKTSGSGKGGGGSLEVLFQGPVRREPKSSDKTHTCPPCPAPPVAGPSVFLFPPKPKDTLMISRTPEVTCVVVGVSHEDPEVKFNWYVDGVEVHNAKTKPREEQYQSTYRVVSVLTVLHQDWLNGKEYKCKVSNKQLPSPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 267 aa, synthetic construct
DFPGHNYTTRNILPGLNITSTFHKTSGSGKGGGGSEPKSSDKTHTSPPSPAPEAARGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - protein, 267 aa, synthetic construct
DFPGHNYTTRNILPGLNITSTFHKTSGSGKGGGGSEPKSSDKTHTCPPCPAPEAARGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 266 aa, synthetic construct
DFPGHNYTTRNILPGLNITSTFHKTSGSGKGGGGSEPKSSDKTHTCPPCPAPPVAGPSVFLFPPKPKDTLMISRTPEVTCVVVGVSHEDPEVKFNWYVDGVEVHNAKTKPREEQYQSTYRVVSVLTVLHQDWLNGKEYKCKVSNKQLPSPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - protein, 267 aa, synthetic construct
DFPGHNYTTRNILPGLNITSTFHKTSGSGKGGGGSEPKSSDKTHTSPPSPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 159 - protein, 232 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 160 - protein, 267 aa, synthetic construct
DFPGHNYTTRNILPGLNITSTFHKTSGSGKGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 161 - protein, 267 aa, synthetic construct; identical to SEQ ID NO: 160

SEQ ID NO: 162 - protein, 232 aa, synthetic construct
EPKSSDKTHTSPPSPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGK